(19) United States
(12) Patent Application Publication
(10) Pub. No.: US 2025/0223313 A1
(43) Pub. Date: Jul. 10, 2025

(54) METHODS OF CONTAMINANT REMOVAL FROM PROTEIN ISOLATES

(71) Applicant: Shattuck Labs, Inc., Austin, TX (US)

(72) Inventors: Taylor SCHREIBER, Austin, TX (US); Joseph TYLER, Austin, TX (US)

(21) Appl. No.: 18/728,568

(22) PCT Filed: Jan. 13, 2023
(86) PCT No.: PCT/US23/10740

§ 371 Date: Jul. 12, 2024

Related U.S. Application Data
(60) Provisional application No. 63/299,701, filed on Jan. 14, 2022.

(51) Int. Cl. C07K 1/22; C07K 16/46

(52) U.S. Cl. CPC C07K 1/22; C07K 16/46; C07K 2317/14; C07K 2317/51; C07K 2317/52; C07K 2317/55; C07K 2317/56; C07K 2319/30

(57) ABSTRACT

The present invention relates, inter alia, to methods for making a variety of proteins, especially proteins having an Fc domain and/or a Fab domain. For instance, the present invention provides methods which overcome common purification challenges.

### PRIORITY

This application claims the benefit of, and priority to, U.S. Provisional Application No. 63/299,701, filed Jan. 14, 2022, the contents of which are hereby incorporated by reference in their entirety.

### TECHNICAL FIELD

The present disclosure relates to methods for purification of proteins, especially the proteins having an Fc domain and/or a Fab domain.

### BACKGROUND

Purified recombinant proteins find use in the laboratory and in the clinic. Generally, recombinant proteins are produced by transfected cells, either mammalian cells or bacterial cells. When purifying recombinant proteins, the quantity and purity of the protein must be sufficient for experimental and/or clinical uses.

Low yields and/or the presence of contaminants are two problems that may occur during purification of recombinant proteins. The contaminants may be derived from the cells used to produce the recombinant protein (including, but not limited to: Chinese hamster ovary cells, human embryonic kidney fibroblasts, Escherichia coli, etc.), and include host cell proteins (HCPs), nucleic acids, lipids, and other cellular material that may be released into the culture media along with the desired recombinant protein. The presence of HCPs in biotherapeutic recombinant proteins destined for the clinic presents a risk to the patient if not mitigated. The risk includes potential immunogenicity (including hypersensitivity to HCPs), which is often unpredictable, side effects caused by the HCPs (e.g., when HCPs have their own biological activities), and decrease in stability or activity of the biotherapeutic (e.g., when HCPs comprise proteases or other enzymes that modify the biotherapeutic). For the proteins used for experimental uses, the risk include data artifacts (e.g. caused by biological activities of HCPs). Thus, methods for improving recombinant protein purification for experimental and/or clinical uses are needed.

### SUMMARY

Accordingly, in various aspects, the present invention provides methods of making recombinant proteins, i.e., chimeric proteins. For instance, the present invention provides methods which overcome common isolation/purification challenges and provide increased yields and decreased amounts of contaminants.

In one aspect, the present disclosure relates to a method for isolating and/or purifying a protein comprising an Fc domain and/or a Fab domain, the method comprising: (a) providing a solution comprising the protein comprising the Fc domain and/or the Fab domain, the solution further comprising one or more host cell proteins (HCPs), (b) contacting the solution comprising the protein comprising the Fc domain and/or the Fab domain with a polypeptide capable of binding clusterin to produce a mixture, and (c) removing free clusterin, clusterin bound to the polypeptide, and/or any additional HCPs contemporaneously bound to clusterin and/or the polypeptide from the mixture, and thereby isolating and/or purifying the protein comprising the Fc domain and/or the Fab domain. In embodiments, the polypeptide capable of binding clusterin is conjugated to a moiety selected from a magnetic bead, a tag, and combination thereof. In embodiments, the clusterin is Cricetulus griseus clusterin.

In embodiments, the polypeptide capable of binding clusterin is conjugated to a magnetic bead and the step (c) comprises: (i) contacting the solution with a magnetic field, and (ii) recovering a second solution comprising the chimeric protein, the second solution being substantially free of the polypeptide capable of binding clusterin and/or the magnetic bead. In embodiments, the polypeptide capable of binding clusterin is conjugated to a tag and the step (c) comprises: (i) contacting the solution with a tag-binding agent, optionally wherein the tag-binding agent is immobilized on a solid support, and (ii) recovering a second solution comprising the chimeric protein, the second solution being substantially free of the polypeptide capable of binding clusterin and/or the tag. In embodiments, the polypeptide capable of binding clusterin is conjugated to a magnetic bead and the step (c) comprises: (i) contacting the solution with a magnetic field, and (ii) recovering a second solution comprising the chimeric protein, the second solution being substantially free of the polypeptide capable of binding clusterin and/or the magnetic bead. In embodiments, the polypeptide capable of binding clusterin is conjugated to a tag and the step (c) comprises: (i) contacting the solution with a tag-binding agent, optionally wherein the tag-binding agent is immobilized on a solid support, and (ii) recovering a second solution comprising the chimeric protein, the second solution being substantially free of the polypeptide capable of binding clusterin and/or the tag. In embodiments, the tag is biotin or an analog thereof and the tag-binding agent is avidin, streptavidin or an analog thereof.

In one aspect, the present disclosure relates to a method for isolating and/or purifying a protein comprising an Fc domain and/or a Fab domain, the method comprising: (a) providing a solution comprising the protein comprising the Fc domain and/or the Fab domain, the solution further comprising one or more host cell proteins (HCP), (b) contacting the solution comprising the protein comprising the Fc domain and/or the Fab domain with a polypeptide immobilized onto the surface of a solid support, wherein the immobilized polypeptide is capable of binding clusterin, and (c) removing free clusterin, clusterin bound to the polypeptide, and any additional HCPs contemporaneously bound to clusterin and/or the polypeptide from the solution, thereby isolating and/or purifying the protein comprising the Fc domain and/or the Fab domain. In embodiments, the step (c) removes at least one HCP. In embodiments, step (c) removes clusterin and at least one more HCP. In embodiments, the method removes clusterin and at least one more HCP. In embodiments, the at least one HCP comprises free clusterin, clusterin bound to the polypeptide, and/or any additional HCPs contemporaneously bound to clusterin and/or the polypeptide. In embodiments, the clusterin is Cricetulus griseus (Chinese hamster) clusterin. In embodiments, the at least one HCP further comprises a protein that directly or indirectly binds clusterin. In embodiments, the polypeptide capable of binding clusterin is directly immobilized to a solid support and the step (c) comprises: (i) passing the solution across the polypeptide immobilized onto the surface of the solid support using buffer conditions that facilitate binding of clusterin to the immobilized polypeptide, and (ii) recovering a protein-containing solution that has passed across the immobilized polypeptide, wherein the recovered protein-containing solution is substantially free of clusterin.

In embodiments, the solution comprising the protein comprising the Fc domain and/or the Fab domain is selected from a culture supernatant, an eluate from a chromatography step, and a cell-free extract. In embodiments, the chromatography step is selected from an affinity chromatography, an ion exchange chromatography, a hydrophobic interaction chromatography, a reverse phase chromatography and a size exclusion chromatography.

In embodiments, the culture supernatant is derived from culture of mammalian cells expressing the protein comprising the Fc domain and/or the Fab domain. In embodiments, the culture supernatant is derived from culturing a cell line expressing the protein comprising the Fc domain and/or the Fab domain. In embodiments, the cell line is selected from NS0 murine myeloma cells, PER.C6 human cells, Chinese hamster ovary (CHO) cells, baby hamster kidney (BHK21) cells, murine myeloma Sp2/0 cells, human embryonic kidney 293 (HEK293) cells, HT-1080 cells, Hela cells, CAP cells, HKB-11 cells, HuH-7 cells, and a derivative thereof. In embodiments, the cell line is selected from CHO DUXB11, CHO DG44, CHOK1, ExpiCHO and Expi293. In embodiments, when the cell line is a Chinese hamster cell line (without limitation, e.g., CHO cell line or a derivative thereof), the at least one HCP removed comprises Cricetulus griseus (Chinese hamster) clusterin.

In embodiments, the protein comprising the Fc domain and/or the Fab domain comprises a mammalian Fc domain. In embodiments, the protein comprising the Fc domain and/or the Fab domain comprises a Fc domain selected from a human Fc domain, a mouse Fc domain, a rat Fc domain, a sheep Fc domain, a goat Fc domain, a donkey Fc domain, a feline Fc domain a hamster Fc domain, a guinea pig Fc domain, a horse Fc domain, a rabbit Fc domain, a pig Fc domain, a dog Fc domain, and a cow Fc domain. In embodiments, the protein comprising the Fc domain and/or the Fab domain comprises a human Fc domain, a human Fab domain and/or a humanized Fab domain.

In embodiments, the protein comprising the Fc domain and/or the Fab domain comprises a Fc domain selected from an IgG Fc domain, an IgA Fc domain an IgM Fc domain, an IgE Fc domain and an IgD Fc domain. In embodiments, the IgG Fc domain is selected from an IgG1 Fc domain, an IgG2 Fc domain, an IgG3 Fc domain, and an IgG4 Fc domain. In embodiments, the IgA is selected from an IgA1 and an IgA2.

In embodiments, the protein comprising the Fc domain and/or the Fab domain is an immunoglobulin. In embodiments, the protein comprising the Fc domain and/or the Fab domain is an antibody, an antibody-like molecule, or a derivative thereof.

In embodiments, the protein comprising the Fc domain and/or the Fab domain is a fusion protein. In embodiments, the fusion protein is a Fab fusion protein. In embodiments, the Fab fusion protein is bispecific or tri-specific. In embodiments, the Fab fusion protein is selected from Fab-scFv, Fab-L-scFv, Fab-H-scFv, tribody, Fab-(scFv)2, a TriFab, a Fab-Fab fusion protein, a scFv-Fc-Fab fusion protein, a bispecific T-cell engager (BiTE), a Fab-Fv fusion protein, and a Fab-dsFv fusion protein.

In embodiments, the fusion protein is an Fc fusion protein. In embodiments, the Fc fusion protein comprises a fusion partner selected from single-chain Fv (scFv), single-chain diabody (scDb), Fv, Fab, and F(ab′)2. In embodiments, the Fc fusion protein comprises the formula: (i) X-Fc, wherein X is selected from an antigen, a mammalian intracellular protein, a mammalian membrane protein, a mammalian secreted protein or a fragment thereof; (ii) Fc-Y, wherein Y is selected from an antigen, a mammalian intracellular protein, a mammalian membrane protein, a mammalian secreted protein or a fragment thereof; or (iii) X-Fc-Y, wherein X and Y are independently selected from an antigen, a mammalian intracellular protein, a mammalian membrane protein, a mammalian secreted protein or a fragment thereof. In embodiments, the fusion protein is capable of modulating an immune response. In embodiments, the fusion protein is a vaccine.

In embodiments, the polypeptide capable of binding clusterin is selected from an antibody, an antibody-like molecule, a recombinant protein, synthetic peptide, eukaryotic peptide, prokaryotic peptide or a binding fragment thereof. In embodiments, the polypeptide capable of binding clusterin is selected from an antibody, a single-domain antibody, a heavy-chain-only antibody (VHH), a single-chain antibody (scFv), or a binding fragment thereof. In embodiments, the polypeptide capable of binding clusterin is a recombinant heavy-chain-only antibody (VHH), or a binding fragment thereof. In embodiments, the VHH, or a binding fragment thereof, is a recombinant protein, a synthetic peptide or a peptide selected from a eukaryotic or prokaryotic peptide library.

In any of the embodiments disclosed herein, the method further comprises at least one more purification step. In embodiments, at least one purification step is liquid chromatography. In embodiments, the chromatography is an affinity chromatography. In embodiments, the affinity chromatography comprises contacting the solution comprising the protein comprising the Fc domain and/or the Fab domain with a moiety having affinity for the X, the Y, the Fc domain and/or the Fab domain. In an illustrative embodiment, the affinity chromatography selected from FcXL chromatography, protein A chromatography, a protein G chromatography, protein L chromatography, and protein M chromatography. In embodiments, the affinity chromatography precedes the above steps that use the polypeptide capable of binding clusterin of any embodiments disclosed herein. In embodiments, the affinity chromatography follows the above steps that use the polypeptide capable of binding clusterin of any embodiments disclosed herein.

In embodiments, the affinity chromatography comprises: contacting a solution comprising the protein comprising the Fc domain and/or the Fab domain with the moiety having affinity for the X, the Y, the Fc domain and/or the Fab domain that is conjugated to a solid support, and thereby directly or indirectly attaching the protein comprising the Fc domain and/or the Fab domain and/or at least one HCP to the solid support. In embodiments, the solid support is a bead or a chromatography resin. In embodiments, the bead is an agarose bead. In embodiments, the agarose bead is an aldehyde-activated agarose bead.

In embodiments, the immobilized polypeptide is coupled to the bead via a free NH₂. In embodiments, the chromatography resin comprises crosslinked poly[styrene divinylbenzene]. In embodiments, the method further comprises contacting the protein comprising the Fc domain and/or the Fab domain that is directly attached to the solid support with a first elution solution that releases the X, the Y, the Fc domain, and/or the Fab domain from the second chromatography column, thereby forming a first eluate which comprises the protein comprising the Fc domain and/or the Fab domain.

In any of the embodiments disclosed herein, the method further comprises at least one more purification step. In embodiments, at least one purification step is liquid chromatography. In embodiments, the chromatography is an ion exchange chromatography. In embodiments, the chromatography is an anion exchange chromatography. In embodiments, the chromatography is a cation exchange chromatography. In embodiments, the ion exchange chromatography comprises: contacting a solution comprising the protein comprising the Fc domain and/or the Fab domain with an ion exchange matrix that is conjugated to a second solid support, thereby directly or indirectly attaching the protein comprising the Fc domain and/or the Fab domain and/or at least one HCP to the second solid support. In embodiments, the solid support is a bead or a chromatography resin. In embodiments, the bead is an agarose bead. In embodiments, the agarose bead is an aldehyde-activated agarose bead. In embodiments, the immobilized polypeptide is coupled to the bead via a free NH₂.

In embodiments, the chromatography resin comprises crosslinked poly[styrene divinylbenzene]. In embodiments, the ion exchange matrix is selected from a weak carboxymethyl cation-exchanger, a strong sulfopropyl (SP) exchanger, and a weak diethylaminoethyl anion exchanger, and a strong quaternary aminoethyl (QAE) exchanger. In embodiments, the ion exchange matrix that is conjugated to the second solid support is located in a third chromatography column. In embodiments, the third chromatography column is washed with a buffer. In embodiments, the method further comprises contacting the protein comprising the Fc domain and/or the Fab domain that is directly attached to the second solid support with a first elution solution that releases the protein comprising the Fc domain and/or the Fab domain from the second chromatography column, thereby forming a second eluate which comprises the protein comprising the Fc domain and/or the Fab domain. In embodiments, the ion exchange chromatography removes at least one HCP. In embodiments, the second eluate comprises fewer contaminants than the solution comprising the protein comprising the Fc domain and/or the Fab domain that was contacted with the ion exchange matrix. In embodiments, the contaminants comprise components of a mammalian cell that harbors a nucleic acid that expresses the protein comprising the Fc domain and/or the Fab domain.

In any of the embodiments disclosed herein, the method further comprises at least one more purification step. In embodiments, at least one purification step is liquid chromatography. In embodiments, the chromatography is a hydrophobic interaction chromatography. In embodiments, the hydrophobic interaction chromatography comprises: contacting a solution comprising the protein comprising the Fc domain and/or the Fab domain with a hydrophobic interaction chromatography (HIC) media that is conjugated to a third solid support, thereby directly or indirectly attaching the protein comprising the Fc domain and/or the Fab domain and/or at least one HCP to the third solid support. In embodiments, the solid support is a bead, a chromatography resin or a membrane. In embodiments, the bead is an agarose bead. In embodiments, the agarose bead is an aldehyde-activated agarose bead. In embodiments, the immobilized polypeptide is coupled to the bead via a free NH₂. In embodiments, the chromatography resin comprises crosslinked poly[styrene divinylbenzene].

In embodiments, the HIC media is selected from MACRO-PREP METHYL, MACRO-PREP T-BUTYL (BIO-RAD), CAPTO PHENYL, CAPTO BUTYL (GE Healthcare), TOYOPEARL HIC (Tosoh), FRACTOGEL EMD PHENYL (Merck Millipore) and a membrane adsorber (e.g., SARTOBIND HIC (Sartorius)). In embodiments, the HIC media that is conjugated to the third solid support is located in a fourth chromatography column or a membrane. In embodiments, the fourth chromatography column or the membrane is washed with a buffer.

In embodiments, the method further comprises contacting the protein comprising the Fc domain and/or the Fab domain that is directly attached to the third solid support with a first elution solution that releases the protein comprising the Fc domain and/or the Fab domain from the third chromatography column, thereby forming a third eluate which comprises the protein comprising the Fc domain and/or the Fab domain. In embodiments, the hydrophobic interaction chromatography removes at least one HCP. In embodiments, the third eluate comprises fewer contaminants than the solution comprising the protein comprising the Fc domain and/or the Fab domain that was contacted with the HIC media that is conjugated to a third solid support. In embodiments, the contaminants comprise components of a mammalian cell that harbors a nucleic acid that expresses the protein comprising the Fc domain and/or the Fab domain.

In one aspect, the present disclosure relates to a method for isolating and/or purifying a protein comprising an Fc domain and/or a Fab domain, the method comprising: (i) a clusterin-scavenging chromatography comprising (a) providing a solution comprising the protein comprising the Fc domain and/or the Fab domain, the solution further comprising one or more host cell proteins (HCP), (b) contacting the solution comprising the protein comprising the Fc domain and/or the Fab domain with a polypeptide immobilized onto the surface of a solid support, wherein the immobilized polypeptide is capable of binding clusterin, and (c) recovering the protein that does not bind to the polypeptide immobilized onto the surface of the solid support, (ii) affinity chromatography selected from FcXL chromatography, protein A chromatography, a protein G chromatography, protein L chromatography, and protein M chromatography, (iii) ion exchange chromatography selected from cation exchange chromatography and anion exchange chromatography, and/or (iv) hydrophobic interaction chromatography and thereby isolating and/or purifying the protein comprising the Fc domain and/or the Fab domain.

In embodiments, the clusterin-scavenging chromatography follows one or more of the affinity chromatography, ion exchange chromatography and hydrophobic interaction chromatography. In embodiments, the clusterin-scavenging chromatography follows the affinity chromatography step. In embodiments, clusterin-scavenging chromatography follows the affinity chromatography step and the ion exchange chromatography step. In embodiments, clusterin-scavenging chromatography follows the affinity chromatography step, the ion exchange chromatography step, and the hydrophobic interaction chromatography step. In embodiments, the clusterin-scavenging chromatography precedes the affinity chromatography step. In embodiments, clusterin-scavenging chromatography precedes the affinity chromatography step and the ion exchange chromatography step. In embodiments, clusterin-scavenging chromatography precedes the affinity chromatography step, the ion exchange chromatography step, and the hydrophobic interaction chromatography step. In embodiments, the affinity chromatography follows the ion exchange chromatography and/or the hydrophobic interaction chromatography. In embodiments, the ion exchange chromatography follows the affinity chromatography and/or the hydrophobic interaction chromatography. In embodiments, the hydrophobic interaction chromatography follows the ion exchange chromatography and/or the affinity chromatography. In embodiments, the method comprises one or more further purification steps.

In embodiments, the step (c) removes at least one HCP. In embodiments, step (c) removes clusterin and at least one more HCP. In embodiments, the at least one HCP comprises free clusterin, clusterin bound to the polypeptide, and/or any additional HCPs contemporaneously bound to clusterin and/or the polypeptide. In embodiments, the clusterin is Cricetulus griseus (Chinese hamster) clusterin. In embodiments, the at least one HCP further comprises a protein that directly or indirectly binds clusterin. In embodiments, the polypeptide capable of binding clusterin is directly immobilized to a solid support and the step (c) comprises: (i) passing the solution across the polypeptide immobilized onto the surface of the solid support using buffer conditions that facilitate binding of clusterin to the immobilized polypeptide, and (ii) recovering a protein-containing solution that has passed across the immobilized polypeptide, wherein the recovered protein-containing solution is substantially free of clusterin.

In one aspect, the present disclosure relates to a method for isolating and/or purifying a protein comprising an Fc domain and/or a Fab domain, the method comprises (i) providing a solution comprising the protein comprising the Fc domain and/or the Fab domain, and performing: (a) a clusterin-scavenging chromatography, (b) an affinity chromatography, (c) an ion exchange chromatography, and/or (d) a hydrophobic interaction chromatography, and thereby isolating and/or purifying the protein comprising the Fc domain and/or the Fab domain.

In embodiments, the clusterin-scavenging chromatography comprises: (a) providing a solution comprising the protein comprising the Fc domain and/or the Fab domain, the solution further comprising one or more host cell proteins (HCP), (b) contacting the solution comprising the protein comprising the Fc domain and/or the Fab domain with a polypeptide immobilized onto the surface of a solid support, wherein the immobilized polypeptide is capable of binding clusterin, and (c) recovering the protein that does not bind to the polypeptide immobilized onto the surface of the solid support. In embodiments, the affinity chromatography uses a moiety having affinity for the X, the Y, the Fc domain and/or the Fab domain. In embodiments, the moiety having affinity for the Fc domain is selected from protein A, a protein G, protein L, and protein M. In embodiments, the ion exchange chromatography is selected from cation exchange chromatography and anion exchange chromatography

In one aspect, the present disclosure relates to a method for isolating and/or purifying a protein comprising an Fc domain and/or a Fab domain, the method comprising: (i) performing a clusterin-scavenging chromatography comprising: (a) providing a solution comprising the protein comprising the Fc domain and/or the Fab domain, the solution further comprising one or more host cell proteins (HCP), (b) contacting the solution comprising the protein comprising the Fc domain and/or the Fab domain with a polypeptide immobilized onto the surface of a solid support, wherein the immobilized polypeptide is capable of binding clusterin, and (c) recovering the protein that does not bind to the polypeptide immobilized onto the surface of the solid support, (ii) performing an affinity chromatography selected from FcXL chromatography, protein A chromatography, a protein G chromatography, protein L chromatography, and protein M chromatography, (iii) performing an ion exchange chromatography selected from cation exchange chromatography and anion exchange chromatography, and/or (iv) performing a hydrophobic interaction chromatography, and thereby isolating and/or purifying the protein comprising the Fc domain and/or the Fab domain.

In embodiments, the clusterin-scavenging chromatography follows one or more of the affinity chromatography, ion exchange chromatography and hydrophobic interaction chromatography. In embodiments, the clusterin-scavenging chromatography precedes one or more of the affinity chromatography, ion exchange chromatography and hydrophobic interaction chromatography.

In embodiments, the method removes clusterin and at least one more HCP. In embodiments, the at least one more HCP is selected from glyceraldehyde-3-phosphate dehydrogenase (GAPDH), actin (e.g., actin, cytoplasmic 2 isoform X2), and serine protease HTRA1 isoform X2 (HTRA).

In embodiments, the method removes at least by 30%, or at least 40%, or at least 40%, or at least 50%, or at least 60%, or at least 70%, or at least 80%, or at least 90% clusterin compared to the solution comprising the protein comprising the Fc domain and/or the Fab domain. In embodiments, the method removes at least by 10%, or at least 20%, or at least 30%, or at least 40%, or at least 50%, or at least 60%, or at least 70% of the at least one more HCP compared to the solution comprising the protein comprising the Fc domain and/or the Fab domain.

An aspect of the present invention provides an isolated protein comprising the Fc domain and/or the Fab domain prepared by methods disclosed herein.

Yet another aspect of the present invention provides a composition comprising the isolated protein comprising the Fc domain and/or the Fab domain prepared by the methods disclosed herein.

In an aspect, the present invention provides a pharmaceutical composition comprising an effective amount of the isolated protein comprising the Fc domain and/or the Fab domain (prepared by methods disclosed herein) and a pharmaceutically acceptable excipient.

In another aspect, the present invention provides an isolated protein comprising the Fc domain and/or the Fab domain prepared according to the methods disclosed herein.

Yet another aspect of the present invention provides a pharmaceutical composition comprising an effective amount of the isolated protein comprising the Fc domain and/or the Fab domain (as prepared by the methods disclosed herein) and a pharmaceutically acceptable excipient.

Yet another aspect of the present invention provides a method for treating a cancer or an autoimmune disease in a subject in need thereof, the method comprising administering to the subject a therapeutically-effective amount of the isolated and/or purified protein comprising the Fc domain and/or the Fab domain of any of the embodiments disclosed herein.

Yet another aspect of the present invention provides a method for treating a cancer or an autoimmune disease in a subject in need thereof, the method comprising administering to the subject a therapeutically-effective amount a protein comprising the Fc domain and/or the Fab domain of any of the embodiments disclosed herein.

Yet another aspect of the present invention provides a method for treating a cancer or an autoimmune disease in a subject in need thereof, the method comprising administering to the subject a therapeutically-effective amount of the composition of any of the embodiments disclosed herein.

Yet another aspect of the present invention provides a method for treating a cancer or an autoimmune disease in a subject in need thereof, the method comprising administering to the subject a therapeutically-effective amount of an isolated and/or purified protein comprising the Fc domain and/or the Fab domain prepared using the method of any of the embodiments disclosed herein.

Yet another aspect of the present invention provides a method for treating a cancer or an autoimmune disease in a subject in need thereof, the method comprising administering to the subject a therapeutically-effective amount of the composition of any of the embodiments disclosed herein.

Yet another aspect of the present invention provides a method for treating a cancer or an autoimmune disease in a subject in need thereof, the method comprising administering to the subject a therapeutically-effective amount of an isolated and/or purified protein comprising the Fc domain and/or the Fab domain prepared using the method of any of the embodiments disclosed herein.

Yet another aspect of the present invention provides a method for preventing or treating an infectious disease in a subject in need thereof, the method comprising administering to the subject a therapeutically-effective amount of the composition of any of the embodiments disclosed herein.

Yet another aspect of the present invention provides a method for preventing or treating an infectious disease in a subject in need thereof, the method comprising administering to the subject a therapeutically-effective amount of an isolated and/or purified protein comprising the Fc domain and/or the Fab domain prepared using the method of any of the embodiments disclosed herein.

Yet another aspect of the present invention provides a method for preventing or treating an infectious disease in a subject in need thereof, the method comprising administering to the subject a therapeutically-effective amount of the composition of any of the embodiments disclosed herein.

Yet another aspect of the present invention provides a method for preventing or treating an infectious disease in a subject in need thereof, the method comprising administering to the subject a therapeutically-effective amount of an isolated and/or purified protein comprising the Fc domain and/or the Fab domain prepared using the method of any of the embodiments disclosed herein. Any aspect or embodiment disclosed herein can be combined with any other aspect or embodiment as disclosed herein.

### BRIEF DESCRIPTION OF THE DRAWINGS

FIG. 1 shows a schematic representation of methods used for isolating and purifying recombinant proteins such as recombinant protein biotherapeutics. Generally, recombinant protein therapeutics are produced by transfected or transduced cells (e.g. NS0 cells, PER.C6 human cells, and Chinese hamster ovary (CHO) cells). These cells are grown in large bioreactors as exemplified by the 2,000 L bioreactor on the left. When the recombinant protein therapeutics is secreted, filtration is employed to remove intact cells and other large organelles or protein complexes, and recover the culture supernatant, as illustrated. Alternatively, a cell-free extract may be used. As illustrated, the culture supernatant is then subjected to one or more chromatography steps to produce a liquid drug substance, which could then be aliquoted into vials and stored as a liquid, frozen or lyophilized drug product.

FIG. 2 shows that a preparation of a chimeric protein comprising an Fc domain prepared using conventional methods comprise many individual CHO host cell proteins (HCPs). Mass spectrometry was performed before and after FcXL chromatography to identify individual HCPs in the purified chimeric protein sample. The abundance of many individual HCPs before and after FcXL affinity chromatography is shown in a representative example.

FIG. 3A and FIG. 3B show that clusterin remains associated with the chimeric protein comprising an Fc domain during further purification. FIG. 3A shows the relative abundance of the indicated individual HCPs in (Panel A) FcXL affinity chromatography eluate, (Panel B and Panel C) Nuvia aPrime 4A hydrophobic anion exchange chromatography eluate after the maximum absorbance detected in the elution peak (Pmax) (Panel B), and across the full elution peak (Panel C), and (Panel D and Panel E) CAPTO MMC multimodal weak cation exchange chromatography in flow through in the presence of 100 mM NaCl (Panel D), or 500 mM NaCl (Panel E). FIG. 3B shows the relative abundance of the indicated individual HCPs during an illustrative elution of the chimeric protein comprising an Fc domain from an anion exchange chromatography process. Total protein concentration, as measured by absorbance at 280 nm is shown with filled diamonds (*), and bars show the amounts of the indicated HCPs at 10, 20, 30, 40 and 50 ml of retention volume. The results indicate a correlation between the concentration of the desired chimeric protein comprising an Fc domain in the eluate and the concentration of certain CHO HCPs, including clusterin.

FIG. 4A and FIG. 4B show a schematic representation of the strategy for the generation of a chromatography resin for the removal of clusterin from the purified preparations of a chimeric protein comprising an Fc domain. FIG. 4A shows the generation of llama anti-clusterin antibodies that are additionally selected to bind clusterin at epitopes that are non-competitive for the epitopes that mediate binding of clusterin to either the Fc and/or Fab domains of the chimeric protein. FIG. 4B shows a schematic representation of a clusterin scavenger resin, and its non-limiting exemplary use for the purification of the chimeric protein comprising an Fc domain from a mixture of free and clusterin-bound-chimeric protein comprising an Fc domain.

FIG. 5A to FIG. 5F demonstrate that the chromatography with clusterin-scavenger resin removes clusterin and other HCPs from the purified preparations of a chimeric protein comprising an Fc domain.

FIG. 5A shows a schematic representation of chromatography with clusterin-scavenger resin. FIG. 5B shows a representative mass spectrometry profile of the load, flow through and eluate from a clusterin-scavenging chromatography run. FIG. 5C shows the amount of clusterin in the load, flow through and eluate from a clusterin-scavenging chromatography run. FIG. 5D shows the relative amounts of the indicated HCPs in FcXL eluate, flow though from the clusterin-scavenging chromatography (“1765-P10 50 gL” and “1765-P10 100 gL”), wash (“1765-P10 W”) and eluate (“1765-P10 S”). FIG. 5E shows the effect of the amount of resin. The amount of the normalized level of HCP when 50, 100 and 150 g/L resin was plotted, with the amount of HCP in FcXL eluate (FLA) was set at 100. FIG. 5F shows exemplary processes for the purification of the chimeric protein.

FIG. 6A and FIG. 6B demonstrate that the purification of a different Fc domain containing protein, the CSF1R-Fc-CD40L chimeric protein, using the clusterin-scavenger chromatography and other steps. FIG. 6A shows the levels of HCP in samples purified using the clusterin-scavenger chromatography (ACR) in comparison with that in the three batches of FcXL chromatography eluates that were loaded onto the clusterin-scavenger resin (Load 1 to Load 3). FIG. 6B shows the levels of in dividual HCPs: clusterin, glyceraldehyde-3-phosphate dehydrogenase (GAPDH), actin, serine protease HTRA1 isoform X2 (HTRA), and three other HCPs (HCP1, HCP2 and HCP3), in FcXL eluate and following the clusterin-scavenger chromatography (ACR), and two further polishing chromatography steps (shown as Polish 1 and Polish 2).

FIG. 7A and FIG. 7B demonstrate that clusterin binds different Fc domain containing proteins. FIG. 7A shows the binding of clusterin to an Fc domain containing protein using a Meso Scale Discovery (MSD)-ELISA-based method. FIG. 7B shows the binding of clusterin to a different Fc domain containing protein using a Meso Scale Discovery (MSD)-ELISA-based method.

### DETAILED DESCRIPTION

The present disclosure is based, in part, on the finding that certain HCPs, including clusterin, account for the major contaminants of in Fc-domain containing chimeric proteins purified using conventional methods used for the purification proteins containing an Fc domain. The present disclosure is also based, in part, on the observation that the use of clusterin-scavenging reagents disclosed herein not only remove clusterin from preparations of chimeric protein containing the HCP contaminants, but also unexpectedly remove other contaminating proteins, thereby increasing purity and decreasing contaminants from the chimeric protein preparations.

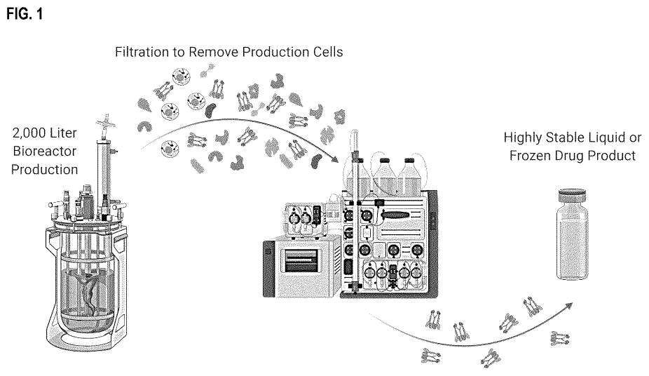

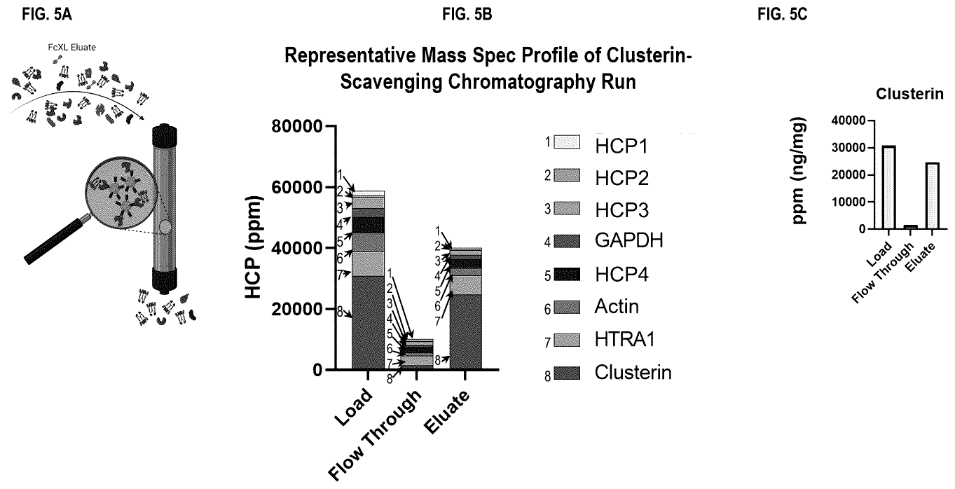

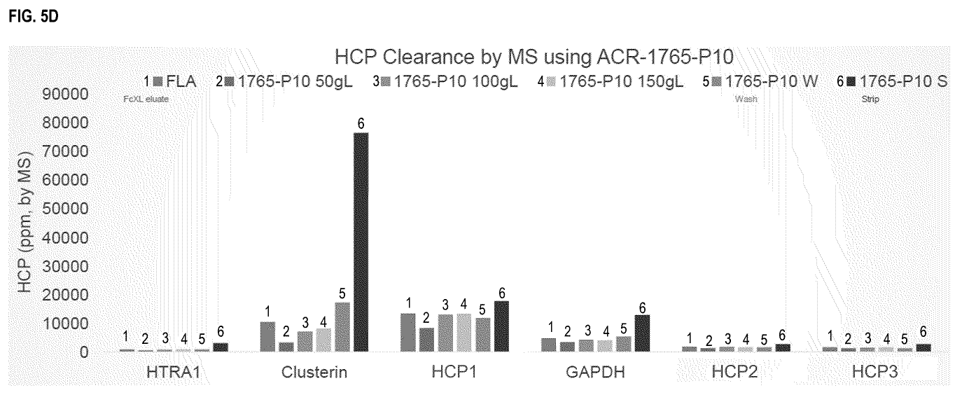

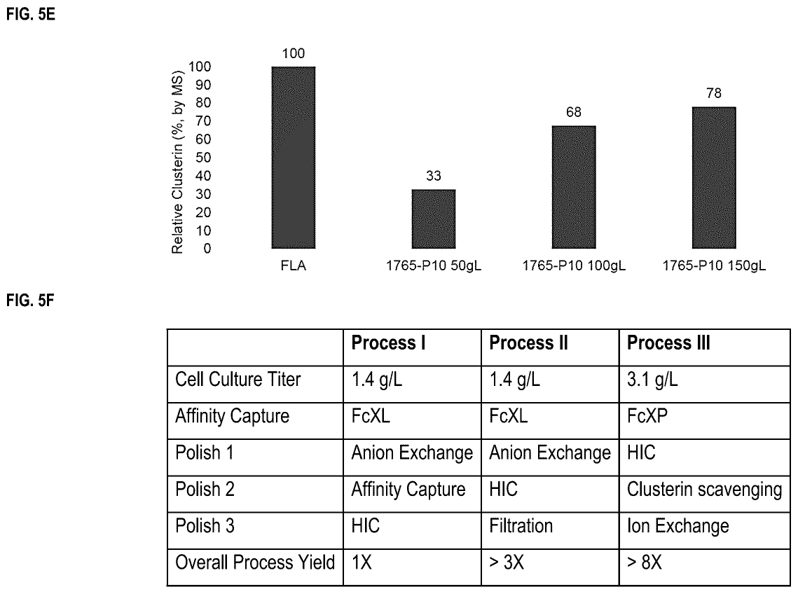

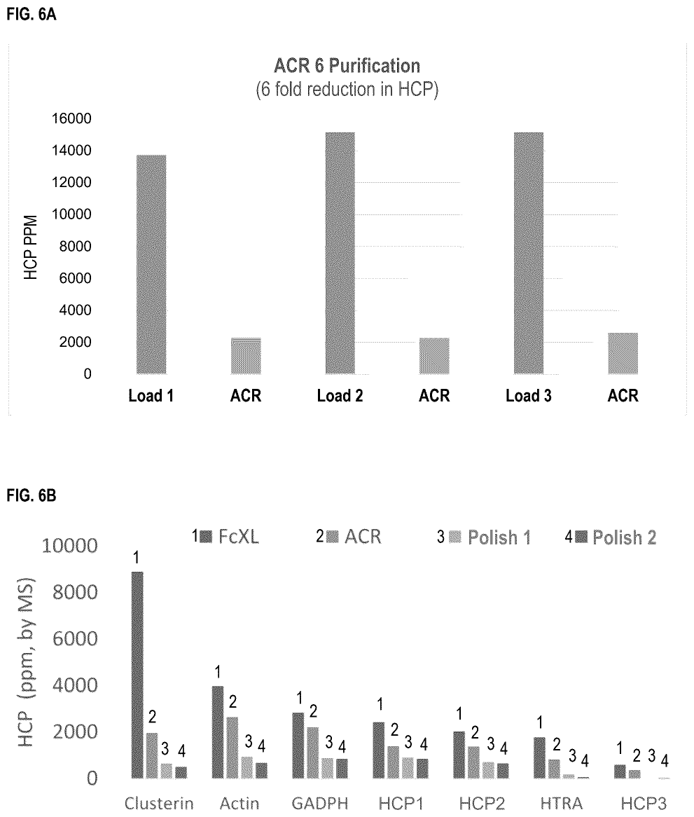

Purification of Fc fusion proteins from mammalian cell expression systems typically involves growth of transfected mammalian cells (e.g., Chinese hamster ovary (CHO) cells and derivatives) in large bioreactors (FIG. 1). Typically, protein A chromatography is used in a typical purification of monoclonal antibodies, and other Fc-domain containing proteins. Although protein A chromatography followed by anion exchange chromatography achieves the removal of a large amount of host cell proteins (HCPs), contaminating HCPs is a major issue faced by this approach. Previous studies have shown that a small number of HCPs that are not related to each other form major contaminants. The especially “difficult to remove” proteins include clusterin, actin, glyceraldehyde-3-phosphate dehydrogenase (GAPDH), serine protease HTRA1 isoform X2, etc. Singh et al., Understanding the mechanism of copurification of “difficult to remove” host cell proteins in rituximab biosimilar products, Biotechnology Progress 36:1-12 (2020); Zhang et al., Comprehensive tracking of host cell proteins during monoclonal antibody purifications using mass spectrometry, mAbs 6(3):659-70 (2014); Levy et al., Identification and characterization of host cell protein product-associated impurities in monoclonal antibody bioprocessing, Biotechnol Bioeng 111(5):904-12 (2014); Zhang et al., Characterization of the co-elution of host cell proteins with monoclonal antibodies during protein A purification, Biotechnol Prog 32(3):708-17 (2016). Therefore, removal of these “difficult to remove” HCPs is a major unresolved problem. Part of the problem lies in the fact that the “difficult to remove” proteins (e.g., clusterin, actin, glyceraldehyde-3-phosphate dehydrogenase (GAPDH) and serine protease HTRA1 isoform X2) are not related to each other and making it hard to evolve strategies to remove each of them. The present disclosure illustrates that the use of clusterin-scavenging reagents disclosed herein not only remove clusterin from preparations of Fc-domain containing chimeric proteins containing the HCP contaminants, but also unexpectedly remove other contaminating proteins (see FIG. 5B, FIG. 5C, FIG. 6A, and FIG. 6B), thereby increasing purity and decreasing contaminants from the Fc-domain containing chimeric proteins preparations.

Accordingly, in one aspect, the present disclosure relates to a method for isolating and/or purifying a protein comprising an Fc domain and/or a Fab domain, the method comprising contacting a solution comprising the protein comprising the Fc domain and/or the Fab domain with a polypeptide capable of binding clusterin. In embodiments, the polypeptide capable of binding clusterin is immobilized on a solid support, thereby allowing easy collection of protein samples, enriched in the proteins comprising an Fc domain and/or a Fab domain, that do not bind or bind with lesser affinity to the polypeptide capable of binding clusterin.

Therapeutic antibodies, Fab-fusion proteins and Fc-fusion proteins are used to treat various diseases, prominent examples include rheumatoid arthritis, psoriasis, multiple sclerosis and many forms of cancer. Therapeutic antibodies can be monoclonal or polyclonal antibodies. Monoclonal antibodies are derived from a single antibody producing cell line, showing identical specificity towards a single antigen. Possible treatments for cancer involve antibodies that are neutralizing tumor cell specific antigens. Approximately 100 monoclonal antibodies have been designated as drugs. They include abciximab (Reopro), adalimumab (Humira, Amjevita), alemtuzumab (Campath), basiliximab (Simulect), cetuximab (Erbitux), inflectra (Remicade), ipilimumab (Yervoy), ixekizumab (Taltz), nivolumab (Opdivo), omalizumab (Xolair), palivizumab (Synagis), panitumumab (Vectibix), pembrolizumab (Keytruda), rituximab (Rituxan), trastuzumab (Herceptin), secukinumab (Cosentyx), and ustekinumab (Stelara). Similarly, Etanercept (Enbrel), alefacept (Amevive), abatacept (Orencia), rilonacept (Arcalyst), romiplostim (Nplate), belatacept (Nulojix), aflibercept (Eylea), ziv-aflibercept (Zaltrap), eftrenonacog-α; BIIB-029 (Alprolix), dulaglutide (Trulicity) and efraloctocog-α (Eloctate) are Fc-fusion proteins. Approved Fab fragments and fusion proteins include abciximab (Reopro), ranibizumab (Lucentis), certolizumab pegol (Cimzia) Catumaxomab and emicizumab.

Manufacturing of antibodies, Fab-fusion proteins and Fc-fusion proteins is typically performed using mammalian cell-based systems. For example, some antibodies, Fab-fusion proteins and Fc-fusion proteins are produced in cultivated mammalian cells with Chinese hamster ovary (CHO) systems. Mammalian systems are often preferred over other hosts, because of their capability for proper protein folding, assembly and posttranslational modification. For example, CHO cell-based systems have the added advantages that they can be grown in suspension at scales required to meet market demands. For example, as shown in FIG. 1, these mammalian cells and their derivative expressing the desired antibodies, Fab-fusion proteins and Fc-fusion proteins are grown in huge bioreactors (fermenters) having thousands of liters capacity. These cells are genetically engineered to express high amounts of recombinant protein. These cells can be grown in serum-free and chemically defined media, making purification easier. Moreover, these cells have less risks of propagating human viruses.

To produce the mammalian cells and their derivative expressing the desired antibodies, Fab-fusion proteins and Fc-fusion proteins, cells are transfected with recombinant vectors expressing a therapeutic protein of interest. Optionally, clones expressing high levels of the antibodies, Fab-fusion proteins and Fc-fusion proteins are isolated and cell lines are established. The methods involved in production of recombinant proteins are discussed by Zhu et al., Industrial Production of Therapeutic Proteins: Cell Lines, Cell Culture, and Purification, Handbook of Industrial Chemistry and Biotechnology ISBN: 978-3-319-52285-2, pp 1639-1669 (2017), the entire contents of which are incorporated herein by reference.

Methods for purifying antibodies, Fab-fusion proteins and Fc-fusion proteins are well established and widespread used. They are employed either alone or in combination. Such methods are, for example, affinity chromatography using microbial-derived proteins or eukaryotic or mammalian derived proteins (e.g., protein A or protein G affinity chromatography, or FcXL affinity chromatography), ion exchange chromatography (e.g. cation exchange using carboxymethyl resins), and hydrophobic interaction or aromatic adsorption chromatography (e.g. with phenyl-sepharose, aza-arenophilic resins, or m-aminophenylboronic acid) form the basis of most methods. These methods are disclosed in US Patent Application Publication Nos: 2019/0218305, 2019/0010188, 2017/0334948, 2017/0320909, 2017/0274299, 2017/0320909, 2016/0115195, 2013/0197200, the contents of which are hereby incorporated by reference in their entireties.

The antibodies, Fab-fusion proteins and Fc-fusion proteins must be purified from any cell-based impurities to an acceptable level before administration in the clinic. One of the greatest challenges is the development of cost effective and efficient processes for purification of proteins on a commercial scale. The use of cell culture supernatants of cells expressing recombinant protein products may contain less impurities if the cells are grown in serum-free medium, the host cell proteins (HCPs) still remain to be eliminated during the purification process. As shown herein, certain HCPs, including clusterin, account for majority of the contaminants of in Fc-domain containing chimeric proteins purified using the conventional methods used for the purification proteins containing an Fc domain. As shown herein, a clusterin-scavenging reagents disclosed herein not only remove clusterin from preparations of chimeric protein containing the HCP contaminants, but also remove other proteins, thereby increasing purity, decreasing contaminants and improving yields.

Clusterin is an extracellular chaperone that binds to a large number of proteins. Clusterin has been shown to bind to a variety of molecules with high affinity including lipids, peptides, and proteins. Bailey et al., Biochemistry 40(39) 11828-11840 (2001). For example, clusterin binds the Fc and Fab regions of IgG. Wilson and Easterbrook-Smith, Biochim Biophys Acta 1159(3):319-26 (1992). As shown herein, clusterin may directly or indirectly binds a large number of proteins. In embodiments, any proteins produced in mammalian cell systems may be purified using clusterin-scavenging chromatography. In embodiments, any protein comprising the Fc domain and/or the Fab domain may be purified using clusterin-scavenging chromatography. In an illustrative embodiment, fusion proteins comprising Fc domain and/or Fab domain, which are discussed in greater details below, may be purified using the clusterin-scavenging reagents disclosed herein.

### Clusterin Scavenging Reagents and Clusterin Scavenging Resin

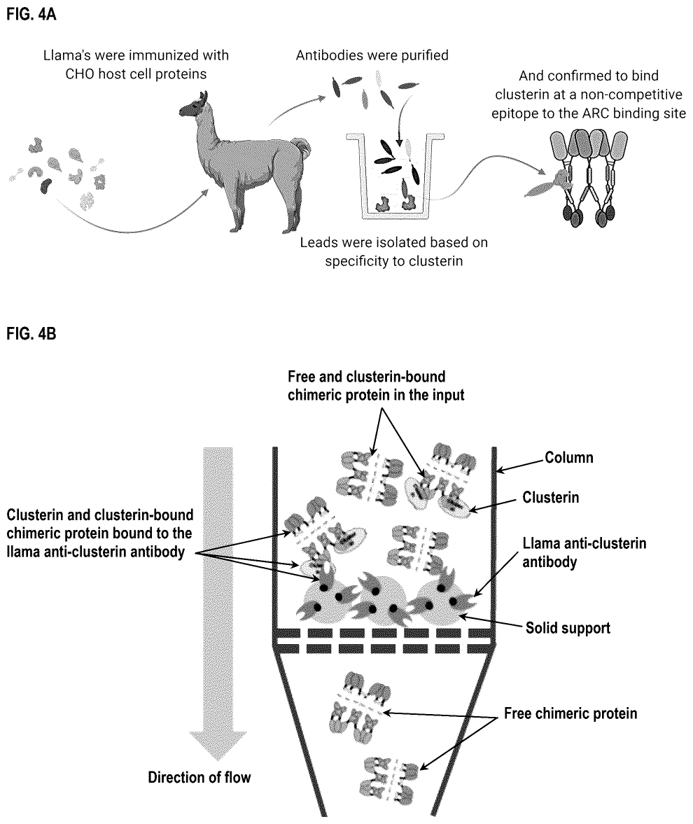

Disclosed herein are polypeptides that are capable of binding clusterin. In embodiments, the polypeptides capable of binding clusterin are utilized in a chromatography scheme as clusterin scavenging reagents. The polypeptide capable of binding clusterin may be conjugated to a moiety such as a tag or a magnetic particle to facilitate removal of clusterin and other HCPs bound to the polypeptide capable of binding clusterin. In embodiments, the polypeptide capable of binding clusterin is immobilized on a solid surface (e.g. beads, membranes and/or a chromatography resin). In these embodiments, clusterin and other HCPs bound to the polypeptide capable of binding clusterin are removed from the protein containing solution by the virtue of their binding to the solid surface, as illustrated in FIG. 4B and FIG. 5A.

In embodiments, the polypeptide capable of binding clusterin is conjugated to a magnetic bead and the step (c) comprises: (i) contacting the solution with a magnetic field, and (ii) recovering a second solution comprising the chimeric protein, the second solution being substantially free of the polypeptide capable of binding clusterin and/or the magnetic bead. In embodiments, the polypeptide capable of binding clusterin is conjugated to a tag and removing the one or more HCPs that bind to the polypeptide capable of binding clusterin from the mixture, and thereby isolating and/or purifying the protein comprising the Fc domain and/or the Fab domain comprises: (i) contacting the solution with a tag-binding agent, optionally wherein the tag-binding agent is immobilized on a solid support, and (ii) recovering a second solution comprising the chimeric protein, the second solution being substantially free of the polypeptide capable of binding clusterin and/or the tag. In embodiments, the tag is biotin or an analog thereof and the tag-binding agent is avidin, streptavidin or an analog thereof. In embodiments, the at least one HCP comprises free clusterin, clusterin bound to the polypeptide, and/or any additional HCPs contemporaneously bound to clusterin and/or the polypeptide. In embodiments, the clusterin is Cricetulus griseus (Chinese hamster) clusterin.

In embodiments, the polypeptide capable of binding clusterin is selected from an antibody, an antibody-like molecule, a recombinant protein, synthetic peptide, eukaryotic peptide, prokaryotic peptide or a binding fragment thereof. In embodiments, the polypeptide capable of binding clusterin is selected from an antibody, a single-domain antibody, a heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂, a peptide mimetic molecule, or a synthetic molecule, as described in U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317. In embodiments, the polypeptide capable of binding clusterin is a binding fragment of the aforementioned molecules. In embodiments, the polypeptide capable of binding clusterin is selected from an antibody, a single-domain antibody, a heavy-chain-only antibody (VHH), a single-chain antibody (scFv), or a binding fragment thereof. In embodiments, the polypeptide capable of binding clusterin is a recombinant heavy-chain-only antibody (VHH), or a binding fragment thereof. In embodiments, the VHH, or a binding fragment thereof, is a recombinant protein, a synthetic peptide or a peptide selected from a eukaryotic or prokaryotic peptide library.

In embodiments, the polypeptide capable of binding clusterin is immobilized onto the surface of a solid support to create a clusterin-scavenging resin or a clusterin-scavenger resin. In embodiments, the solid support is a bead, a membrane, or a chromatography resin. In embodiments, the bead is an agarose bead. In embodiments, the agarose bead is an aldehyde-activated agarose bead. In embodiments, the immobilized polypeptide was coupled to the bead via a free NH₂. In embodiments, the chromatography resin comprises crosslinked poly[styrene divinylbenzene]. In embodiments, the clusterin scavenging resin is used for scavenging clusterin and associated proteins (e.g., HCPs) from a solution comprising a protein comprising an Fc domain and/or a Fab domain (e.g. antibodies, Fab-fusion proteins and Fc-fusion proteins) to purify these proteins. In embodiments, the clusterin scavenging resin is used for scavenging clusterin and associated proteins (e.g., HCPs) from a solution comprising a protein comprising a chimeric protein disclosed herein, and to thereby purify these proteins. In embodiments, the clusterin scavenging resin removes clusterin and other HCPs that associate with and/or form complex with clusterin.

As examples, in embodiments, a binding affinity of clusterin for the polypeptide capable of binding clusterin is less than 100 nM, is less than 90 nM, is less than 80 nM, is less than 70 nM, is less than 60 nM, is less than 50 nM, is less than 40 nM, is less than 30 nM, is less than 20 nM, is less than 10 nM, is less than 5 nM, is less than 1 nM, is less than 900 pM, is less than 800 pM, is less than 700 pM, is less than 600 pM, is less than 500 pM, is less than 400 pM, is less than 300 pM, is less than 200 pM, is less than 100 pM, is less than 90 pM, is less than 80 pM, is less than 70 pM, is less than 60 pM, is less than 50 pM, is less than 40 pM, is less than 30 pM, is less than 20 pM, is less than 10 pM, is less than 5 pM, or is less than 1 pM.

As examples, in embodiments, the polypeptide capable of binding clusterin may bind clusterin at any pH from 1 to 14. In embodiments, the affinity for the clusterin binding polypeptide is optimal within a pH range of about 3 to about 11. In embodiments, the affinity for the clusterin binding polypeptide is optimal within a pH range of about 4 to about 9.5. In embodiments, the affinity for the clusterin binding polypeptide is optimal within a pH range of about 5.5 to about 8.0. In embodiments, the clusterin-binding polypeptide binds clusterin optimally at pH of about 5, or about 5.5, or about 6, or about 6.5, or about 7, or about 7.5, or about 8. In embodiments, the clusterin-binding polypeptide efficiently binds clusterin at pH of less than about 7, or less than about 6, or less than about 5, or less than about 4, or less than about 3. In embodiments, the clusterin-binding polypeptide efficiently binds clusterin at pH of more than about 6, or more than about 7, or more than about 8, or more than about 9, or more than about 10, or more than about 11.

As examples, in embodiments, the polypeptide capable of binding clusterin may bind clusterin at any salt concentration from about 5 mM to about 5 M. In embodiments, the affinity for the clusterin binding polypeptide is optimal within salt concentration of 20 mM to about 1 M. In embodiments, the affinity for the clusterin binding polypeptide is optimal within salt concentration of 50 mM to about 500 mM. In embodiments, the affinity for the clusterin binding polypeptide is optimal within salt concentration of 100 mM to about 250 mM. In embodiments, the clusterin-binding polypeptide binds clusterin efficiently at a salt concentration of less than about 2 M, or less than about 1 M, or less than about 500 mM, or less than about 250 mM, or less than about 100 mM, or less than about 50 mM, or less than about 25 mM. In embodiments, the clusterin-binding polypeptide binds clusterin efficiently at a salt concentration of more than about 50 mM, or more than about 100 mM, or more than about 250 mM, or more than about 500 mM, or more than about 1 M, or more than about 2 M.

As examples, in embodiments, the polypeptide capable of binding clusterin may bind clusterin at any pH from 1 to 14. In embodiments, the affinity for the clusterin binding polypeptide is optimal within a pH range of about 3 to about 11. In embodiments, the affinity for the clusterin binding polypeptide is optimal within a pH range of about 4 to about 9.5. In embodiments, the affinity for the clusterin binding polypeptide is optimal within a pH range of about 5.5 to about 8.0. In embodiments, the clusterin-binding polypeptide binds clusterin optimally at pH of about 5, or about 5.5, or about 6, or about 6.5, or about 7, or about 7.5, or about 8. In embodiments, the clusterin-binding polypeptide efficiently binds clusterin at pH of less than about 7, or less than about 6, or less than about 5, or less than about 4, or less than about 3. In embodiments, the clusterin-binding polypeptide efficiently binds clusterin at pH of more than about 6, or more than about 7, or more than about 8, or more than about 9, or more than about 10, or more than about 11.

### Clusterin Scavenging Chromatography

Disclosed herein are methods of scavenging clusterin and associated proteins (e.g., HCPs) from a solution comprising a protein comprising an Fc domain and/or a Fab domain (e.g. antibodies, Fab-fusion proteins and Fc-fusion proteins) to purify these proteins. These methods are also referred to herein as clusterin-scavenging chromatography.

In one aspect, the present disclosure relates to a method for isolating and/or purifying a protein comprising an Fc domain and/or a Fab domain, the method comprising: (a) providing a solution comprising the protein comprising the Fc domain and/or the Fab domain, the solution further comprising one or more host cell proteins (HCPs), (b) contacting the solution comprising the protein comprising the Fc domain and/or the Fab domain with a polypeptide capable of binding clusterin, and (c) removing the one or more HCPs that bind to the polypeptide capable of binding clusterin from the solution, and thereby isolating and/or purifying the protein comprising the Fc domain and/or the Fab domain. In embodiments, the one or more HCPs that bind to the polypeptide capable of binding clusterin are free clusterin, clusterin bound to the polypeptide, and/or any additional HCPs contemporaneously bound to clusterin and/or the polypeptide from the mixture. In these embodiments, the polypeptide capable of binding clusterin is not immobilized on a solid surface. In these embodiments, clusterin is removed using secondary reagents. In embodiments, the polypeptide capable of binding clusterin is conjugated to a moiety selected from a magnetic bead, a tag, and combination thereof. In embodiments, the polypeptide capable of binding clusterin is conjugated to a magnetic bead and the method further comprises (d) contacting the solution with a magnetic field, and (e) recovering a second solution comprising the protein comprising the Fc domain and/or the Fab domain, the second solution being substantially free of the polypeptide capable of binding clusterin and/or the magnetic bead. In embodiments, the clusterin is Cricetulus griseus (Chinese hamster) clusterin.

In one aspect, the present disclosure relates to a method for isolating and/or purifying a protein comprising an Fc domain and/or a Fab domain, the method comprising: (a) providing a solution comprising the protein comprising the Fc domain and/or the Fab domain, the solution further comprising one or more host cell proteins (HCP), (b) contacting the solution comprising the protein comprising the Fc domain and/or the Fab domain with a polypeptide immobilized onto the surface of a solid support, wherein the immobilized polypeptide is capable of binding clusterin, and (c) removing free clusterin, clusterin bound to the polypeptide, and any additional HCPs contemporaneously bound to clusterin and/or the polypeptide from the solution, thereby isolating and/or purifying the protein comprising the Fc domain and/or the Fab domain. In embodiments, the step (c) removes at least one HCP. In embodiments, step (c) removes clusterin and at least one more HCP. In embodiments, the at least one HCP comprise free clusterin, clusterin bound to the polypeptide, and/or any additional HCPs contemporaneously bound to clusterin and/or the polypeptide. In embodiments, the clusterin is Cricetulus griseus (Chinese hamster) clusterin. In embodiments, the polypeptide capable of binding clusterin is directly immobilized to a solid support and the step (c) comprises: (i) passing the solution across the polypeptide immobilized onto the surface of the solid support using buffer conditions that facilitate binding of clusterin to the immobilized polypeptide, and (ii) recovering a protein-containing solution that has passed across the immobilized polypeptide, wherein the recovered protein-containing solution is substantially free of clusterin.

In one aspect, the present disclosure relates to a method for isolating and/or purifying a chimeric protein from a solution, wherein the chimeric protein has the structure selected from: X-Linker, and Linker-Y, wherein the X and the Y are selected from a mammalian secreted protein, mammalian intracellular protein, a mammalian membrane protein, an antigen, or a fragment thereof, and the linker comprises an Fc domain, the method comprising: (a) providing a solution comprising the chimeric protein, the solution further comprising one or more host cell proteins (HCPs), (b) contacting the solution comprising the chimeric protein with a polypeptide capable of binding clusterin to produce a mixture, and (c) removing the polypeptide capable of binding clusterin from the mixture, and thereby removing free clusterin, clusterin bound to the polypeptide, and/or any additional HCPs contemporaneously bound to clusterin and/or the polypeptide from the mixture, and isolating and/or purifying the chimeric protein. In embodiments, the polypeptide capable of binding clusterin is conjugated to a moiety selected from a magnetic bead, a tag, and combination thereof.

In one aspect, the present disclosure relates to a method for isolating and/or purifying a chimeric protein from a solution, wherein the chimeric protein has the structure: X-Linker-Y wherein X and Y are independently absent or selected from a mammalian secreted protein, mammalian intracellular protein, a mammalian membrane protein, an antigen, or a fragment thereof, and the linker comprises an Fc domain, the method comprising: (a) providing a solution comprising the chimeric protein, the solution further comprising one or more host cell proteins (HCPs), (b) contacting the solution comprising the chimeric protein with a polypeptide capable of binding clusterin to produce a mixture, and (c) removing the polypeptide capable of binding clusterin from the mixture, and thereby removing free clusterin, clusterin bound to the polypeptide, and/or any additional HCPs contemporaneously bound to clusterin and/or the polypeptide from the mixture, and thereby isolating and/or purifying the chimeric protein. In embodiments, the polypeptide capable of binding clusterin is conjugated to a moiety selected from a magnetic bead, a tag, and combination thereof.

In embodiments, the polypeptide capable of binding clusterin is conjugated to a magnetic bead and the step (c) comprises: (i) contacting the solution with a magnetic field, and (ii) recovering a second solution comprising the chimeric protein, the second solution being substantially free of the polypeptide capable of binding clusterin and/or the magnetic bead. In embodiments, the polypeptide capable of binding clusterin is conjugated to a tag and the method further comprises: (i) contacting the solution with a tag-binding agent, optionally wherein the tag-binding agent is immobilized on a solid support, and (ii) recovering a second solution comprising the chimeric protein, the second solution being substantially free of the polypeptide capable of binding clusterin and/or the tag.

Clusterin-scavenging chromatography may be first step, the last step or a step between other purification steps. Accordingly, in embodiments, clusterin-scavenging chromatography is the first chromatography step carried out during the method for isolating and/or purifying a protein comprising an Fc domain and/or a Fab domain. In embodiments, clusterin-scavenging chromatography follows one or more additional chromatography steps including but not limited to selected from an affinity chromatography, an ion exchange chromatography, a hydrophobic interaction chromatography, a reverse phase chromatography and a size exclusion chromatography. In embodiments, clusterin-scavenging chromatography follows an affinity chromatography step. In embodiments, clusterin-scavenging chromatography follows an affinity chromatography step and an ion exchange chromatography step, which may precede or follow the affinity chromatography step. In embodiments, clusterin-scavenging chromatography follows an affinity chromatography step, an ion exchange chromatography step, and a hydrophobic interaction chromatography, which may precede or follow the affinity chromatography step and the ion exchange chromatography step, or follow both the affinity chromatography step and the ion exchange chromatography step, or follow one of (and precede the other of) the affinity chromatography and the ion exchange chromatography steps.

Any suitable solution comprising a protein comprising an Fc domain and/or a Fab domain may be used as the starting material for clusterin-scavenging chromatography. In embodiments, the solution comprising the protein comprising the Fc domain and/or the Fab domain is selected from a culture supernatant, an eluate or flow through or fraction (that comprises the protein comprising the Fc domain and/or the Fab domain) of a chromatography step, and a cell-free extract. In embodiments, the chromatography step is selected from an affinity chromatography, an ion exchange chromatography, a hydrophobic interaction chromatography, a reverse phase chromatography and a size exclusion chromatography. In embodiments, the chromatography step is an affinity chromatography. In embodiments, the chromatography step is an ion exchange chromatography. In embodiments, the chromatography step is a hydrophobic interaction chromatography.

In embodiments, the culture supernatant is derived from culture of mammalian cells expressing the protein comprising the Fc domain and/or the Fab domain. In embodiments, the culture supernatant is derived from culturing a cell line expressing the protein comprising the Fc domain and/or the Fab domain. In embodiments, the cell line is selected from NS0 murine myeloma cells, PER.C6 human cells, Chinese hamster ovary (CHO) cells, baby hamster kidney (BHK21) cells, murine myeloma Sp2/0 cells, human embryonic kidney 293 (HEK293) cells, HT-1080 cells, Hela cells, CAP cells, HKB-11 cells, HuH-7 cells, and a derivative thereof. In embodiments, the cell line is selected from CHO DUXB11, CHO DG44, CHOK1, ExpiCHO and Expi293. In embodiments, the at least one HCP that the clusterin-scavenging chromatography removes comprises free clusterin, clusterin bound to the polypeptide, and/or any additional HCPs contemporaneously bound to clusterin and/or the polypeptide. In embodiments, when the cell line is a Chinese hamster cell line (without limitation, e.g. CHO cell line or a derivative thereof), the clusterin is Cricetulus griseus (Chinese hamster) clusterin.

In embodiments, the cell-free extract is derived from culture of mammalian cells expressing the protein comprising the Fc domain and/or the Fab domain. In embodiments, the cell-free extract is derived from culturing a cell line expressing the protein comprising the Fc domain and/or the Fab domain. In embodiments, the cell line is selected from NS0 murine myeloma cells, PER.C6 human cells, Chinese hamster ovary (CHO) cells, baby hamster kidney (BHK21) cells, murine myeloma Sp2/0 cells, human embryonic kidney 293 (HEK293) cells, HT-1080 cells, Hela cells, CAP cells, HKB-11 cells, HuH-7 cells, and a derivative thereof. In embodiments, the cell line is selected from CHO DUXB11, CHO DG44, CHOK1, ExpiCHO and Expi293. In embodiments, the at least one HCP that the clusterin-scavenging chromatography removes comprises free clusterin, clusterin bound to the polypeptide, and/or any additional HCPs contemporaneously bound to clusterin and/or the polypeptide. In embodiments, when the cell line is a Chinese hamster cell line (without limitation, e.g. CHO cell line or a derivative thereof), the clusterin is Cricetulus griseus (Chinese hamster) clusterin.

In embodiments, when the polypeptide capable of binding clusterin is immobilized onto the surface of a solid support, a flow through fraction from clusterin-scavenging chromatography comprises the protein comprising the Fc domain and/or the Fab domain. In embodiments, when the polypeptide capable of binding clusterin is not immobilized onto the surface of a solid support, a fraction that is substantially free of the protein comprising the Fc domain comprises the protein comprising the Fc domain and/or the Fab domain.

In embodiments, the polypeptide is immobilized on a chromatography resin. In embodiments, the chromatography resin is located in a chromatography column. In embodiments, the flow through sample of step (c) is recovered. In embodiments, optionally the chromatography column is washed and a second flow through sample is recovered. In embodiments, the flow through sample and the second flow through sample are mixed to produce a combined flow through sample. In embodiments, the step (c) removes at least one HCP. In embodiments, the at least one HCP comprises clusterin. In embodiments, the at least one HCP further comprises a contaminant that binds to clusterin. In embodiments, step (c) removes clusterin and at least one more HCP. In embodiments, the flow through sample and the second flow through sample and/or the combined flow through sample comprises fewer contaminants than the solution comprising chimeric protein. In embodiments, the contaminants comprise components of a mammalian cell harboring a nucleic acid that expresses the chimeric protein. In embodiments, the polypeptide capable of binding clusterin is directly immobilized to a solid support and the step (c) comprises: (i) passing the solution across the polypeptide immobilized onto the surface of the solid support using buffer conditions that facilitate binding of clusterin to the immobilized polypeptide, and (ii) recovering a protein-containing solution that has passed across the immobilized polypeptide, wherein the recovered protein-containing solution is substantially free of clusterin.

In embodiments, the solid support is a bead or a chromatography resin. In embodiments, the bead is an agarose bead. In embodiments, the agarose bead is an aldehyde-activated agarose bead. In embodiments, the immobilized polypeptide is coupled to the bead via a free NH₂. In embodiments, the chromatography resin comprises crosslinked poly[styrene divinylbenzene].

In embodiments, the solution comprising the chimeric protein is selected from an eluate from a chromatography step, a culture supernatant, and a cell-free extract. In embodiments, the chromatography step is selected from an affinity chromatography, an ion exchange chromatography, a hydrophobic interaction chromatography, a reverse phase chromatography and a size exclusion chromatography. In embodiments, the chromatography step is an affinity chromatography. In embodiments, the chromatography step is an ion exchange chromatography. In embodiments, the chromatography step is a hydrophobic interaction chromatography.

In embodiments, the culture supernatant is derived from culture of mammalian cells expressing chimeric protein. In embodiments, the culture supernatant is derived from culturing a cell line expressing chimeric protein. In embodiments, the cell line is selected from NS0 murine myeloma cells, PER.C6 human cells, and Chinese hamster ovary (CHO) cells, baby hamster kidney (BHK21) cells, murine myeloma Sp2/0 cells, human embryonic kidney 293 (HEK293) cells, HT-1080 cells, Hela cells, CAP cells, HKB-11 cells, HuH-7 cells, Sf9 cells, and a derivative thereof. In embodiments, the cell line is selected from CHO DUXB11, CHO DG44, CHOK1, ExpiCHO and Expi293. In embodiments, the at least one HCP that the clusterin-scavenging chromatography removes comprises free clusterin, clusterin bound to the polypeptide, and/or any additional HCPs contemporaneously bound to clusterin and/or the polypeptide. In embodiments, when the cell line is a Chinese hamster cell line (without limitation, e.g. CHO cell line or a derivative thereof), the clusterin is Cricetulus griseus (Chinese hamster) clusterin.

In embodiments, the cell-free extract is derived from culture of mammalian cells expressing chimeric protein. In embodiments, the cell-free extract is derived from culturing a cell line expressing chimeric protein. In embodiments, the cell line is selected from NS0 murine myeloma cells, PER.C6 human cells, and Chinese hamster ovary (CHO) cells, baby hamster kidney (BHK21) cells, murine myeloma Sp2/0 cells, human embryonic kidney 293 (HEK293) cells, HT-1080 cells, Hela cells, CAP cells, HKB-11 cells, HuH-7 cells, Sf9 cells, and a derivative thereof. In embodiments, the cell line is selected from CHO DUXB11, CHO DG44, CHOK1, ExpiCHO and Expi293. In embodiments, the at least one HCP that the clusterin-scavenging chromatography removes comprises free clusterin, clusterin bound to the polypeptide, and/or any additional HCPs contemporaneously bound to clusterin and/or the polypeptide. In embodiments, when the cell line is a Chinese hamster cell line (without limitation, e.g. CHO cell line or a derivative thereof), the clusterin is Cricetulus griseus (Chinese hamster) clusterin.

Proteins that May be Purified Using Clusterin Scavenging Chromatography

Clusterin is an extracellular chaperone that binds to a large number of proteins. Clusterin has been shown to bind to a variety of molecules with high affinity including lipids, peptides, and proteins. Bailey et al., Biochemistry 40 (39) 11828-11840 ( ) 2001). For example, clusterin binds the Fc and Fab regions of IgG. Wilson and Easterbrook-Smith, Biochim Biophys Acta 1159(3):319-26 (1992). As shown herein, clusterin may directly or indirectly binds a large number of proteins. In embodiments, any proteins produced in mammalian cell systems may be purified using clusterin-scavenging chromatography. In embodiments, protein comprising the Fc domain and/or the Fab domain may be purified using clusterin-scavenging chromatography.

In embodiments, the protein comprising the Fc domain and/or the Fab domain comprises a mammalian Fc domain. In embodiments, the protein comprising the Fc domain and/or the Fab domain comprises a Fc domain selected from a human Fc domain, a mouse Fc domain, a rat Fc domain, a sheep Fc domain, a goat Fc domain, a donkey Fc domain, a feline Fc domain a hamster Fc domain, a guinea pig Fc domain, a horse Fc domain, a rabbit Fc domain, a pig Fc domain, a dog Fc domain, and a cow Fc domain. In embodiments, the protein comprising the Fc domain and/or the Fab domain comprises a human Fc domain, a human Fab domain and/or a humanized Fab domain.

In embodiments, the protein comprising the Fc domain and/or the Fab domain comprises a Fc domain selected from an IgG Fc domain, an IgA Fc domain an IgM Fc domain, an IgE Fc domain and an IgD Fc domain. In embodiments, the IgG Fc domain is selected from an IgG1 Fc domain, an IgG2 Fc domain, an IgG3 Fc domain, and an IgG4 Fc domain. In embodiments, the IgA is selected from an IgA1 and an IgA2.

In embodiments, the protein comprising the Fc domain and/or the Fab domain is an immunoglobulin. In embodiments, the protein comprising the Fc domain and/or the Fab domain is an antibody, an antibody-like molecule, or a derivative thereof. In embodiments, the derivative of the antibody is selected from Fab, Fd, F(ab′)2, Fab′, and Fv, or a binding fragment thereof. In embodiments, the derivative of the antibody-like molecule is selected from a single-domain antibody, a heavy-chain-only antibody (VHH), a single-chain antibody (scFv), scFv, ScFv-Fc, a diabody, a ScFv-CH, a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody or a binding fragment thereof. These types of binding agents are disclosed In U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317.

In embodiments, the Fc domain is a mammalian Fc domain. In embodiments, the chimeric protein comprises a Fc domain selected from a human Fc domain, a mouse Fc domain, a rat Fc domain, a sheep Fc domain, a goat Fc domain, a donkey Fc domain, a feline Fc domain a hamster Fc domain, a guinea pig Fc domain, a horse Fc domain, a rabbit Fc domain, a pig Fc domain, a dog Fc domain, and a cow Fc domain. In embodiments, the chimeric protein comprises a human Fc domain. In embodiments, the chimeric protein comprises a Fc domain selected from an IgG Fc domain, an IgA Fc domain, an IgM Fc domain, an IgE Fc domain and an IgD Fc domain. In embodiments, the IgG Fc domain is selected from an IgG1 Fc domain, an IgG2 Fc domain, an IgG3 Fc domain, and an IgG4 Fc domain. In embodiments, the IgA is selected from an IgA1 and an IgA2.

In one aspect, the present disclosure provides a method for isolating and/or purifying a chimeric protein from a solution, wherein the chimeric protein has the structure selected from: X-Linker and Linker-Y wherein the X or the Y is selected from a mammalian secreted protein, mammalian intracellular protein, a mammalian membrane protein, an antigen, or a fragment thereof, and the linker comprises an Fc domain, the method comprising: (a) providing a solution comprising the chimeric protein, the solution further comprising one or more host cell proteins (HCPs), (b) contacting the solution comprising the chimeric protein with a polypeptide capable of binding clusterin to produce a mixture, and (c) removing the polypeptide capable of binding clusterin from the mixture, and thereby removing free clusterin, clusterin bound to the polypeptide, and/or any additional HCPs contemporaneously bound to clusterin and/or the polypeptide from the mixture, and thereby isolating and/or purifying the chimeric protein. In embodiments, the polypeptide capable of binding clusterin is conjugated to a tag and the method further comprises (i) contacting the solution with a tag-binding agent, optionally wherein the tag-binding agent is immobilized on a solid support, and (ii) recovering a second solution comprising the chimeric protein, the second solution being substantially free of the polypeptide capable of binding clusterin and/or the tag.

In one aspect, the present disclosure provides a method for isolating and/or purifying a chimeric protein from a solution, wherein the chimeric protein has the structure: X-Linker-Y wherein the X and the Y are independently absent or selected from a mammalian secreted protein, mammalian intracellular protein, a mammalian membrane protein, an antigen, or a fragment thereof, and the linker comprises an Fc domain, the method comprising: (a) providing a solution comprising the chimeric protein, the solution further comprising one or more host cell proteins (HCPs), (b) contacting the solution comprising the chimeric protein with a polypeptide capable of binding clusterin to produce a mixture, and (c) removing the polypeptide capable of binding clusterin from the mixture, and thereby removing free clusterin, clusterin bound to the polypeptide, and/or any additional HCPs contemporaneously bound to clusterin and/or the polypeptide from the mixture, and thereby isolating and/or purifying the chimeric protein. In embodiments, the polypeptide capable of binding clusterin is conjugated to a moiety selected from a magnetic bead, a tag, and combination thereof. In embodiments, the polypeptide capable of binding clusterin is conjugated to a magnetic bead and the step (c) comprises: (i) contacting the solution with a magnetic field, and (ii) recovering a second solution comprising the chimeric protein, the second solution being substantially free of the polypeptide capable of binding clusterin and/or the magnetic bead. In embodiments, the polypeptide capable of binding clusterin is conjugated to a tag and the method further comprises (i) contacting the solution with a tag-binding agent, optionally wherein the tag-binding agent is immobilized on a solid support, and (ii) recovering a second solution comprising the chimeric protein, the second solution being substantially free of the polypeptide capable of binding clusterin and/or the tag.

In one aspect, the present disclosure provides a method for isolating and/or purifying a chimeric protein from a solution, wherein the chimeric protein has the structure selected from: X-Linker and Linker-Y wherein the X and the Y is selected from a mammalian secreted protein, mammalian intracellular protein, a mammalian membrane protein, or a fragment thereof, and the linker comprises an Fc domain, the method comprising: (a) providing a solution comprising the chimeric protein, the solution further comprising one or more host cell proteins (HCP), (b) contacting the solution comprising the chimeric protein with a polypeptide immobilized onto the surface of a solid support, wherein the immobilized polypeptide is capable of binding clusterin, and (c) removing free clusterin, clusterin bound to the polypeptide, and any additional HCPs contemporaneously bound to clusterin and/or the polypeptide from the solution, thereby isolating and/or purifying the chimeric protein.

In one aspect, the present disclosure provides a method for isolating and/or purifying a chimeric protein from a solution, wherein the chimeric protein has the structure: X-Linker-Y wherein X and Y are independently absent or selected from a mammalian secreted protein, mammalian intracellular protein, a mammalian membrane protein, or a fragment thereof, and the linker comprises an Fc domain, the method comprising: (a) providing a solution comprising the chimeric protein, the solution further comprising one or more host cell proteins (HCP), (b) contacting the solution comprising the chimeric protein with a polypeptide immobilized onto the surface of a solid support, wherein the immobilized polypeptide is capable of binding clusterin, and (c) removing free clusterin, clusterin bound to the polypeptide, and any additional HCPs contemporaneously bound to clusterin and/or the polypeptide from the solution, thereby isolating and/or purifying the chimeric protein.

In embodiments, the X and/or Y is independently an antigen, a mammalian intracellular protein, a mammalian secreted protein, a mammalian membrane protein, or a fragment thereof. In embodiments, the X and/or Y is an antigen, wherein the antigen is derived from a pathogen. In embodiments, the pathogen is selected from a virus, a bacterium, a protozoan, a parasite, and a fungus. In embodiments, the antigen is a cancer antigen. In embodiments, the cancer antigen is a neoantigen.

In embodiments, the X and/or Y is a mammalian intracellular protein, or a fragment thereof.

In embodiments, the X and/or Y is a mammalian secreted protein, or a fragment thereof. In embodiments, the secreted protein is a cytokine. In embodiments, the cytokine is selected from IFN-α, IFN-β, IFN-ε, IFN-κ, IFN-ω IFN-γ, IL-1α, IL-1β, IL-1, IL-2, IL-3, IL-4, IL-5, IL-6, IL-7, IL-9, IL-10, IL-11, IL-12, IL-13, IL-15, IL-16, IL-17, IL-18, IL-19, IL-20, IL-35, TNF, leukemia inhibitory factor (LIF), oncostatin M (OSM), granulocyte colony-stimulating factor (G-CSF), TNF-α, TNF-β, lymphotoxin (LT)-β, LIGHT, Fas ligand (FasL)/CD178, TGF-β1, TGF-β2, TGF-β3, XCL1, XCL2, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CXCL1, CXCL2, CXCL3, CXCL4, CXCL5, CXCL6, CXCL7, CXCL8, CXCL9, CXCL10, CXCL11, CXCL12, CXCL13, CXCL14, CX3CL1, Epo, Tpo, SCF, and FLT-3L.

In embodiments, the X and/or Y is a mammalian membrane protein, or a fragment thereof. In embodiments, the mammalian membrane protein is selected from SLAMF4, IL-2Rα, 4-1BB/TNFRSF9, IL-2R β, ALCAM, B7-1, IL-4 R, B7-H3, BLAME/SLAMFS, CEACAM1, IL-6 R, IL-7 Rα, IL-10Rα, IL-10 R β, IL-12R β 1, IL-12Rβ2, CD2, IL-13Rα1, IL-13, CD3, CD4, ILT2/CDS5j, ILT3/CDS5k, ILT4/CDS5d, ILT5/CDS5a, Integrin α 4/CD49d, CDS, Integrin α E/CD103, CD6, Integrin α M/CD11b, CDS, Integrin α X/CD11c, Integrin R2/CDIS, KIR/CD15S, CD27/TNFRSF7, KIR2DL1, CD2S, KIR2DL3, CD30/TNFRSFS, KIR2DL4/CD15Sd, CD31/PECAM-1, KIR2DS4, CD40 Ligand/TNFSF5, LAG-3, CD43, LAIR1, CD45, LAIR2, CDS3, Leukotriene B4-R1, CDS4/SLAMF5, NCAM-L1, CD94, NKG2A, CD97, NKG2C, CD229/SLAMF3, NKG2D, CD2F-10/SLAMF9, NT-4, CD69, NTB-A/SLAMF6, Common γ Chain/IL-2 R γ, Osteopontin, CRACC/SLAMF7, PD-1, CRTAM, PSGL-1, CTLA-4, RANK/TNFRSF11A, CX3CR1, CX3CL1, L-Selectin, SIRP p 1, SLAM, TCCR/WSX-1, DNAM-1, Thymopoietin, EMMPRIN/CD147, TIM-1, EphB6, TIM-2, Fas/TNFRSF6, TIM-3, Fas Ligand/TNFSF6, TIM-4, Fcγ RIII/CD16, TIM-6, TNFR1/TNFRSF1A, Granulysin, TNF RIII/TNFRSF1B, TRAIL RI/TNFRSFIOA, ICAM-1/CD54, TRAIL R2/TNFRSF10B, ICAM-2/CD102, TRAILR3/TNFRSF10C, IFN-γR1, TRAILR4/TNFRSF10D, IFN-γ R2, TSLP, IL-1 R1 and TSLP R, or a fragment thereof.

In embodiments, the X is a mammalian membrane protein is a Type I membrane protein, or a fragment thereof. In embodiments, the Type I membrane protein is selected from TIM-3, BTLA, PD-1, CTLA-4, LAG-3, CD244, CSF1R, CD160, TIGIT, SIRPa/CD172a, 2B4, VISTA, VSIG8, LAG3, CD200, B7-H3, BTNL3, BTNL8, BTN2A1, BTN3A1, BTN3A2, NKG2A, NKG2B, NKG2C, NKG2D, NKG2E, NKG2F, and NKG2H, and TMIGD2, or a fragment thereof. In embodiments, the fragment of the Type I membrane protein is the extracellular domain or the ligand binding portion thereof.

In embodiments, the Y is a mammalian membrane protein is a Type II membrane protein, or a fragment thereof. In embodiments, the Type II membrane protein is selected from OX-40 ligand (OX-40L), LIGHT (CD258), GITR ligand (GITRL), 4-1BB Ligand (4-1BBL), CD70, CD30 ligand (CD30L), CD40 ligand (CD40L), a C-type lectin domain (CLEC) family member, CD137 ligand, RANKL, TRAIL, FasL, TL1A, CD80, CD86, CD58, PD-1, SLAMF6, SIRPα and TGFBR2, or a fragment thereof. In embodiments, the fragment of the Type II membrane protein is the extracellular domain thereof. In embodiments, the Type II membrane protein is the ligand binding portion thereof.

In embodiments, the protein comprising the Fc domain and/or the Fab domain is a fusion protein. In embodiments, the fusion protein is a Fab fusion protein. In embodiments, the Fab fusion protein is bispecific or tri-specific. In embodiments, the Fab fusion protein is selected from Fab-scFv, Fab-L-scFv, Fab-H-scFv, tribody, Fab-(scFv)2, a TriFab, a Fab-Fab fusion protein, a scFv-Fc-Fab fusion protein, a bispecific T-cell engager (BiTE), a Fab-Fv fusion protein, and a Fab-dsFv fusion protein.

In embodiments, the fusion protein is an Fc fusion protein. In embodiments, the Fc fusion protein comprises a fusion partner selected from single-chain Fv (scFv), single-chain diabody (scDb), Fv, Fab, and F(ab′)2. In embodiments, the Fc fusion protein comprises the formula: (i) X-Fc, wherein X is selected from an antigen, a mammalian intracellular protein, a mammalian membrane protein, a mammalian secreted protein or a fragment thereof; (ii) Fc-Y, wherein Y is selected from an antigen, a mammalian intracellular protein, a mammalian membrane protein, a mammalian secreted protein or a fragment thereof; or (iii) X-Fc-Y, wherein X and Y are independently selected from an antigen, a mammalian intracellular protein, a mammalian membrane protein, a mammalian secreted protein or a fragment thereof.

In embodiments, the X and/or the Y are an antigen or a fragment thereof. In embodiments, the antigen is derived from a pathogen. In embodiments, the pathogen is selected from a virus, a bacterium, a protozoan, a parasite, and a fungus. In embodiments, the antigen is a cancer antigen. In embodiments, the cancer antigen is a neoantigen.

In embodiments, the X and/or the Y are a mammalian intracellular protein or a fragment thereof.

In embodiments, a mammalian secreted protein or a biologically active fragment thereof. In embodiments, the mammalian secreted protein is a coagulation factor, a cytokine or a serum protein. In embodiments, the cytokine is selected from IFN-α, IFN-β, IFN-ε, IFN-κ, IFN-ω IFN-γ, IL-1α, IL-1β, IL-1, IL-2, IL-3, IL-4, IL-5, IL-6, IL-7, IL-9, IL-10, IL-11, IL-12, IL-13, IL-15, IL-16, IL-17, IL-18, IL-19, IL-20, IL-35, TNF, leukemia inhibitory factor (LIF), oncostatin M (OSM), granulocyte colony-stimulating factor (G-CSF), TNF-α, TNF-β, lymphotoxin (LT)-β, LIGHT, Fas ligand (FasL)/CD178, TGF-β1, TGF-β2, TGF-β3, XCL1, XCL2, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CXCL1, CXCL2, CXCL3, CXCL4, CXCL5, CXCL6, CXCL7, CXCL8, CXCL9, CXCL10, CXCL11, CXCL12, CXCL13, CXCL14, CX3CL1, Epo, Tpo, SCF, and FLT-3L.

In embodiments, the X and/or the Y are a mammalian membrane protein, or a fragment thereof. In embodiments, the mammalian membrane protein is selected from SLAMF4, IL-2Rα, 4-1BB/TNFRSF9, IL-2R β, ALCAM, B7-1, IL-4 R, B7-H3, BLAME/SLAMFS, CEACAM1, IL-6 R, IL-7 Rα, IL-10Rα, IL-10 R 3, IL-12R β 1, IL-12Rβ2, CD2, IL-13Rα1, IL-13, CD3, CD4, ILT2/CDS5j, ILT3/CDS5k, ILT4/CDS5d, ILT5/CDS5a, Integrin α 4/CD49d, CDS, Integrin α E/CD103, CD6, Integrin α M/CD11b, CDS, Integrin α X/CD11c, Integrin R2/CDIS, KIR/CD15S, CD27/TNFRSF7, KIR2DL1, CD2S, KIR2DL3, CD30/TNFRSFS, KIR2DL4/CD15Sd, CD31/PECAM-1, KIR2DS4, CD40 Ligand/TNFSF5, LAG-3, CD43, LAIR1, CD45, LAIR2, CDS3, Leukotriene B4-R1, CDS4/SLAMF5, NCAM-L1, CD94, NKG2A, CD97, NKG2C, CD229/SLAMF3, NKG2D, CD2F-10/SLAMF9, NT-4, CD69, NTB-A/SLAMF6, Common γ Chain/IL-2 R γ, Osteopontin, CRACC/SLAMF7, PD-1, CRTAM, PSGL-1, CTLA-4, RANK/TNFRSF11A, CX3CR1, CX3CL1, L-Selectin, SIRP β1, SLAM, TCCR/WSX-1, DNAM-1, Thymopoietin, EMMPRIN/CD147, TIM-1, EphB6, TIM-2, Fas/TNFRSF6, TIM-3, Fas Ligand/TNFSF6, TIM-4, Fcγ RIII/CD16, TIM-6, TNFR1/TNFRSF1A, Granulysin, TNF RIII/TNFRSF1B, TRAIL RI/TNFRSFIOA, ICAM-1/CD54, TRAIL R2/TNFRSF10B, ICAM-2/CD102, TRAILR3/TNFRSF10C, IFN-γR1, TRAILR4/TNFRSF10D, IFN-γ R2, TSLP, IL-1 R1 and TSLP R, or a fragment thereof.

In embodiments, the X and/or Y is independently a mammalian membrane protein, or a fragment thereof. In embodiments, the X is a Type I membrane protein, or a fragment thereof. In embodiments, the Type I membrane protein is selected from TIM-3, BTLA, PD-1, CTLA-4, LAG-3, CD244, CSF1R, CD160, TIGIT, SIRPa/CD172a, 2B4, VISTA, VSIG8, LAG3, CD200, B7-H3, BTNL3, BTNL8, BTN2A1, BTN3A1, BTN3A2, NKG2A, NKG2B, NKG2C, NKG2D, NKG2E, NKG2F, and NKG2H, and TMIGD2, or a fragment thereof. In embodiments, the fragment of the Type I membrane protein is the extracellular domain or the ligand binding portion thereof. In embodiments, the Y is a mammalian membrane protein is a Type II membrane protein, or a fragment thereof. In embodiments, the Type II membrane protein is selected from OX-40 ligand (OX-40L), LIGHT (CD258), GITR ligand (GITRL), 4-1BB Ligand (4-1BBL), CD70, CD30 ligand (CD30L), CD40 ligand (CD40L), a C-type lectin domain (CLEC) family member, CD137 ligand, RANKL, TRAIL, FasL, TL1A, CD80, CD86, CD58, PD-1, SLAMF6, SIRPα and TGFBR2, or a fragment thereof.

In embodiments, the Type I transmembrane protein is selected from PD1, TIM3, CD172a(SIRPα), TIGIT, CD115 (CSF1R), BTLA, TMIGD2, and VSIG8, or a variant thereof. In embodiments, the Type II transmembrane protein is selected from OX40L, CD40L, LIGHT (CD258), 4 1BBL (CD137L), GITRL, TL1A, CD30L, LIGHT, and CD70, or a variant thereof. In embodiments, the Type I transmembrane protein is selected from PD1, TIM3, CD172a(SIRPα), TIGIT, CD115 (CSF1R), BTLA, TMIGD2, and VSIG8, or a variant thereof; and the Type II transmembrane protein is selected from OX40L, CD40L, LIGHT (CD258), 4 1BBL (CD137L), GITRL, TL1A, CD30L, LIGHT, and CD70, or a variant thereof.

In embodiments, the Type I transmembrane protein is PD1, or a variant thereof, and the Type II transmembrane protein is selected from OX40L, CD40L, LIGHT (CD258), 4 1BBL (CD137L), GITRL, TL1A, CD30L, and CD70, or a variant thereof. In embodiments, the Type I transmembrane protein is TIM 3, or a variant thereof, and the Type II transmembrane protein is selected from OX40L, CD40L, LIGHT (CD258), 4-1BBL (CD137L), GITRL, TL1A, CD30L, LIGHT, or CD70, or a variant thereof. In embodiments, the Type I transmembrane protein is CD172a(SIRPα), or a variant thereof, and the Type II transmembrane protein is selected from OX40L, CD40L, LIGHT (CD258), 4 1BBL (CD137L), TL1A, CD30L, or CD70, or a variant thereof. In embodiments, the Type I transmembrane protein is TIGIT, or a variant thereof, and the Type II transmembrane protein is selected from OX40L, CD40L, LIGHT (CD258), 4-1BBL (CD137L), GITRL, TL1A, CD30L, LIGHT, or CD70, or a variant thereof. In embodiments, the Type I transmembrane protein is CD115 (CSF1R), or a variant thereof, and the Type II transmembrane protein is selected from OX40L, CD40L, LIGHT (CD258), 4 1BBL (CD137L), TL1A, CD30L, or CD70, or a variant thereof. In embodiments, the Type I transmembrane protein is BTLA, or a variant thereof, and the Type II transmembrane protein is selected from OX40L, CD40L, LIGHT (CD258), 4-1BBL (CD137L), GITRL, TL1A, CD30L, LIGHT, or CD70, or a variant thereof. In embodiments, the Type I transmembrane protein is TMIGD2, or a variant thereof, and the Type II transmembrane protein is selected from OX40L, CD40L, LIGHT (CD258), 4-1BBL (CD137L), GITRL, TL1A, CD30L, LIGHT, or CD70, or a variant thereof. In embodiments, the Type I transmembrane protein is VSIG8, or a variant thereof, and the Type II transmembrane protein is selected from OX40L, CD40L, LIGHT (CD258), 4-1BBL (CD137L), GITRL, TL1A, CD30L, LIGHT, or CD70, or a variant thereof.

In embodiments, the Type I transmembrane protein is PD-1 and the Type II transmembrane protein is OX40L; the Type I transmembrane protein is PD-1 and the Type II transmembrane protein is GITRL; the Type I transmembrane protein is PD-1 and the Type II transmembrane protein is 4-1 BBL; the Type I transmembrane protein is PD-1 and the Type II transmembrane protein is LIGHT; the Type I transmembrane protein is PD-1 and the Type II transmembrane protein is TL1A; the Type I transmembrane protein is SIRPα/CD172a and the Type II transmembrane protein is CD40L; the Type I transmembrane protein is SIRPα/CD172a and the Type II transmembrane protein is OX40L; the Type I transmembrane protein is SIRPα/CD172a and the Type II transmembrane protein is LIGHT; the Type I transmembrane protein is BTLA and the Type II transmembrane protein is OX40L; the Type I transmembrane protein is TMIGD2 and the Type II transmembrane protein is OX40L; the Type I transmembrane protein is TIM3 and the Type II transmembrane protein is OX40L; the Type I transmembrane protein is TIM3 and the Type II transmembrane protein is CD40L; the Type I transmembrane protein is VSIG8 and the Type II transmembrane protein is OX40L; the Type I transmembrane protein is VSIG8 and the Type II transmembrane protein is 4-1 BBL; the Type I transmembrane protein is VSIG8 and the Type II transmembrane protein is CD30L; the Type I transmembrane protein is CSF1R and the Type II transmembrane protein is CD40L; the Type I transmembrane protein is TIGIT and the Type II transmembrane protein is 4-1 BBL; the Type I transmembrane protein is TIGIT and the Type II transmembrane protein is GITRL; the Type I transmembrane protein is TIGIT and the Type II transmembrane protein is LIGHT; the Type I transmembrane protein is TIGIT and the Type II transmembrane protein is OX40L; or the Type I transmembrane protein is TIGIT and the Type II transmembrane protein is TL1A.

In embodiments, the Type I transmembrane protein is selected from CD86, CD80, CD48, PD-1, SIRPα, SLAMF6, and TGFBR; and wherein the Type II transmembrane protein is NKG2A. In embodiments, the Type I transmembrane protein is TGFBR2, and the Type II transmembrane protein is selected from 4-1BB Ligand (4-1BBL), CD30 Ligand (CD30L) and an NKG2 receptor. In embodiments, the Type I transmembrane protein is FLT3L, and the Type II transmembrane protein is selected from CD40L, 4-1BBL, OX40L, and GITRL.

In embodiments, the fragment of the Type II membrane protein is the extracellular domain or a ligand binding portion thereof. In embodiments, the fusion protein is selected from PD1-Fc-OX40L, PD1-Fc-GITRL, SIRPα/CD172a-Fc-CD40L, CD172a-Fc-OX40L, PD1-Fc-TL1A, BTLA-Fc-OX40L, TMIGD2-Fc-OX40L, TIM3-Fc-OX40L, TIM3-Fc-CD40L, PD1-Fc-4-1 BBL, CD172a-Fc-LIGHT, VSIG8-Fc-4-1 BBL, VSIG8-Fc-CD30L, VSIG8-Fc-CD40L, VSIG8-Fc-FasL, VSIG8-Fc-GITRL, VSIG8-FC-LIGHT, VSIG8-Fc-TL1A, and VSIG8-Fc-TRAIL, CSF1R-Fc-CD40L, TIGIT-Fc-4-1 BBL, TIGIT-Fc-GITRL, TIGIT-Fc-LIGHT, TIGIT-Fc-OX40L, TIGIT-Fc-TL1A, PD-1-Fc-LIGHT, CD86-Fc-NKG2A, CD80-Fc-NKG2A, CD48-Fc-NKG2A, PD-1-Fc-NKG2A, SLAMF6-Fc-NKG2A, SIRPα-Fc-NKG2A, TGFBR2-Fc-NKG2A, TGFBR2-Fc-4-1BBL, BTNL2A1/BTNL3A1-Fc-scFv, BTNL3A1/BTNL3A2-Fc-scFv, BTNL3A1/BTNL3A3-Fc-scFv, BTNL3/BTNL8-Fc-scFv.

In embodiments, the chimeric protein comprises an extracellular domain from BTLA and an extracellular domain from OX40L, e.g., BTLA-Fc-OX40L.

In embodiments, the chimeric protein comprises an extracellular domain from CSF1R and an extracellular domain from CD40L, e.g., CSF1R-Fc-CD40L.

In embodiments, the chimeric protein comprises an extracellular domain from PD-1 and an extracellular domain from CD40L, e.g., PD-1-Fc-CD40L.

In embodiments, the chimeric protein comprises an extracellular domain from PD1 and an extracellular domain from LIGHT, e.g., PD1-Fc-LIGHT.

In embodiments, the chimeric protein comprises an extracellular domain from PD-1 and an extracellular domain from 4-1BBL, e.g., PD-1-Fc-4-1BBL.

In embodiments, the chimeric protein comprises an extracellular domain from PD-1 and an extracellular domain from GITRL, e.g., PD-1-Fc-GITRL.

In embodiments, the chimeric protein comprises an extracellular domain from PD-1 and an extracellular domain from OX40L, e.g., PD-1-Fc-OX40L.

In embodiments, the chimeric protein comprises an extracellular domain from PD-1 and an extracellular domain from TL1A, e.g., PD-1-Fc-TL1A.

In embodiments, the chimeric protein comprises an extracellular domain from SIRPα and an extracellular domain from LIGHT, e.g., SIRP1a-Fc-LIGHT.

In embodiments, the chimeric protein comprises an extracellular domain from SIRPα and an extracellular domain from CD40L, e.g., SIRPα-Fc-CD40L.

In embodiments, the chimeric protein comprises an extracellular domain from SIRPα and an extracellular domain from OX40L, e.g., SIRPα-Fc-OX40L.

In embodiments, the chimeric protein comprises an extracellular domain from TIGIT and an extracellular domain from LIGHT, e.g., TIGIT-Fc-LIGHT.

In embodiments, the chimeric protein comprises an extracellular domain from TIGIT and an extracellular domain from OX40L, e.g., TIGIT-Fc-OX40L.

In embodiments, the chimeric protein comprises an extracellular domain from TIM-3 and an extracellular domain from CD40L, e.g., TIM-3-Fc-CD40L.

In embodiments, the chimeric protein comprises an extracellular domain from TIM3 and an extracellular domain from OX40L, e.g., TIM3-Fc-OX40L.

In embodiments, the chimeric protein comprises an extracellular domain from TMIGD2 and an extracellular domain from OX40L, e.g., TMIGD2-Fc-OX40L.

In embodiments, the chimeric protein comprises an extracellular domain from VSIG8 and an extracellular domain from OX40L, e.g., VSIG8-Fc-OX40L. In embodiments, a chimeric protein is capable of binding murine ligand(s)/receptor(s).

In embodiments, a chimeric protein is capable of binding human ligand(s)/receptor(s).

Exemplary proteins that may be purified using clusterin-scavenging chromatography are disclosed in PCT International Publication Nos. WO 2017/059168, WO 2018/157163, WO 2018/157164, WO 2018/157165, WO 2018/157162, WO 2019/246508, WO 2020/047325, WO 2020/047327, WO 2020/047328, WO 2020/047329, WO 2020/047319, WO 2020/047322, WO 2020/146393, WO 2020/176718, WO 2020/232365, the contents of each of which are hereby incorporated by reference in their entireties.

In embodiments, the fusion protein is capable of modulating an immune response. In embodiments, the fusion protein is a vaccine.

In embodiments, each X domain and Y domain of the chimeric protein is independently capable of binding to its cognate receptor or ligand with a K_{D} of about 1 nM to about 5 nM, for example, about 1 nM, about 1.5 nM, about 2 nM, about 2.5 nM, about 3 nM, about 3.5 nM, about 4 nM, about 4.5 nM, or about 5 nM.

In embodiments, the chimeric protein binds to a cognate receptor or ligand with a K_{D} of about 5 nM to about 15 nM, for example, about 5 nM, about 5.5 nM, about 6 nM, about 6.5 nM, about 7 nM, about 7.5 nM, about 8 nM, about 8.5 nM, about 9 nM, about 9.5 nM, about 10 nM, about 10.5 nM, about 11 nM, about 11.5 nM, about 12 nM, about 12.5 nM, about 13 nM, about 13.5 nM, about 14 nM, about 14.5 nM, or about 15 nM.

In embodiments, each X domain and Y domain of the chimeric protein is independently capable of binding to its cognate receptor or ligand with a K_{D} of less than about 1 pM, about 900 nM, about 800 nM, about 700 nM, about 600 nM, about 500 nM, about 400 nM, about 300 nM, about 200 nM, about 150 nM, about 130 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the chimeric protein binds to human cognate receptor or ligand with a K_{D} of less than about 1 nM, about 900 pM, about 800 pM, about 700 pM, about 600 pM, about 500 pM, about 400 pM, about 300 pM, about 200 pM, about 100 pM, about 90 pM, about 80 pM, about 70 pM, about 60 pM about 55 pM about 50 pM about 45 pM, about 40 pM, about 35 pM, about 30 pM, about 25 pM, about 20 pM, about 15 pM, or about 10 pM, or about 1 pM (as measured, for example, by surface plasmon resonance or biolayer interferometry).

In embodiments, wherein the Linker domain is a polypeptide selected from a flexible amino acid sequence, an IgG hinge region, and an antibody sequence.

In embodiments, the Linker domain comprising at least one cysteine residue capable of forming a disulfide bond. The at least one cysteine residue is capable of forming a disulfide bond between a pair (or more) of chimeric proteins. Without wishing to be bound by theory, such disulfide bond forming is responsible for maintaining a useful multimeric state of chimeric proteins. This allows for efficient production of the chimeric proteins; it allows for desired activity in vitro and in vivo.

In embodiments, the Linker domain is derived from naturally-occurring multi-domain proteins or is an empirical linker as described, for example, in Chichili et al., (2013), Protein Sci. 22(2):153-167, Chen et al., (2013), Adv Drug Deliv Rev. 65(10):1357-1369, the entire contents of which are hereby incorporated by reference. In embodiments, the Linker domain may be designed using linker designing databases and computer programs such as those described in Chen et al., (2013), Adv Drug Deliv Rev. 65(10):1357-1369 and Crasto et. al., (2000), Protein Eng. 13(5):309-312, the entire contents of which are hereby incorporated by reference.

In embodiments, the Linker domain comprises a protein chain. In embodiments, the protein chain is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long.

In embodiments, the Linker domain is flexible.

In embodiments, the Linker domain is rigid.

In embodiments, the Linker domain is substantially comprised of glycine and serine residues (e.g., about 30%, or about 40%, or about 50%, or about 60%, or about 70%, or about 80%, or about 90%, or about 95%, or about 97%, or about 98%, or about 99%, or about 100% glycines and serines).

In embodiments, the Linker domain comprises a hinge region of an antibody (e.g., of IgG, IgA, IgD, and IgE, inclusive of subclasses (e.g., IgG1, IgG2, IgG3, and IgG4, and IgA1, and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2. In embodiments, the linker may be derived from human IgG4 and contain one or more mutations to enhance dimerization (including S228P) or FcRn binding.

According to crystallographic studies, the immunoglobulin hinge region can be further subdivided functionally into three regions: the upper hinge region, the core region, and the lower hinge region. See Shin et al., 1992 Immunological Reviews 130:87. The upper hinge region includes amino acids from the carboxyl end of C_{H1} to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the C_{H2} domain and includes residues in C_{H2}. Id. The core hinge region of wild-type human IgG1 contains a cysteine-proline palindrome sequence which, when dimerized by disulfide bond formation, results in a cyclic octapeptide believed to act as a pivot, thus conferring flexibility. In embodiments, the present linker comprises, one, or two, or three of the upper hinge region, the core region, and the lower hinge region of any antibody (e.g., of IgG, IgA, IgD, and IgE, inclusive of subclasses (e.g., IgG1, IgG2, IgG3, and IgG4, and IgA1 and IgA2)). The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region protein chain to intestinal proteases, considered an advantageous property for a secretory immunoglobulin. In embodiments, the linker of the present invention comprises one or more glycosylation sites.

In embodiments, the Linker domain comprises an Fc domain of an antibody (e.g., of IgG, IgA, IgD, and IgE, inclusive of subclasses (e.g., IgG1, IgG2, IgG3, and IgG4, and IgA1 and IgA2)).

In a chimeric protein of the present invention, the Linker domain comprises a hinge-CH2-CH3 Fc domain derived from IgG4. In embodiments, the Linker domain comprises a hinge-CH2-CH3 Fc domain derived from a human IgG4.

In embodiments, the Linker domain comprises a hinge-CH2-CH3 Fc domain derived from a human IgG1 antibody. In embodiments, the Fc domain exhibits increased affinity for and enhanced binding to the neonatal Fc receptor (FcRn). In embodiments, the Fc domain includes one or more mutations that increases the affinity and enhances binding to FcRn. Without wishing to be bound by theory, it is believed that increased affinity and enhanced binding to FcRn increases the in vivo half-life of the present chimeric proteins.

In embodiments, the Fc domain in a Linker domain contains one or more amino acid substitutions at amino acid residue 250, 252, 254, 256, 308, 309, 311, 416, 428, 433 or 434 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference), or equivalents thereof. In embodiments, the amino acid substitution at amino acid residue 250 is a substitution with glutamine. In embodiments, the amino acid substitution at amino acid residue 252 is a substitution with tyrosine, phenylalanine, tryptophan or threonine. In embodiments, the amino acid substitution at amino acid residue 254 is a substitution with threonine. In embodiments, the amino acid substitution at amino acid residue 256 is a substitution with serine, arginine, glutamine, glutamic acid, aspartic acid, or threonine. In embodiments, the amino acid substitution at amino acid residue 308 is a substitution with threonine. In embodiments, the amino acid substitution at amino acid residue 309 is a substitution with proline. In embodiments, the amino acid substitution at amino acid residue 311 is a substitution with serine. In embodiments, the amino acid substitution at amino acid residue 385 is a substitution with arginine, aspartic acid, serine, threonine, histidine, lysine, alanine or glycine. In embodiments, the amino acid substitution at amino acid residue 386 is a substitution with threonine, proline, aspartic acid, serine, lysine, arginine, isoleucine, or methionine. In embodiments, the amino acid substitution at amino acid residue 387 is a substitution with arginine, proline, histidine, serine, threonine, or alanine. In embodiments, the amino acid substitution at amino acid residue 389 is a substitution with proline, serine or asparagine. In embodiments, the amino acid substitution at amino acid residue 416 is a substitution with serine. In embodiments, the amino acid substitution at amino acid residue 428 is a substitution with leucine. In embodiments, the amino acid substitution at amino acid residue 433 is a substitution with arginine, serine, isoleucine, proline, or glutamine. In embodiments, the amino acid substitution at amino acid residue 434 is a substitution with histidine, phenylalanine, or tyrosine.

In embodiments, the Fc domain linker (e.g., comprising an IgG constant region) comprises one or more mutations such as substitutions at amino acid residue 252, 254, 256, 433, 434, or 436 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). In embodiments, the IgG constant region includes a triple M252Y/S254T/T256E mutation or YTE mutation. In embodiments, the IgG constant region includes a triple H433K/N434F/Y436H mutation or KFH mutation. In embodiments, the IgG constant region includes an YTE and KFH mutation in combination.

In embodiments, the linker comprises an IgG constant region that contains one or more mutations at amino acid residues 250, 253, 307, 310, 380, 428, 433, 434, and 435 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). Illustrative mutations include T250Q, M428L, T307A, E380A, 1253A, H310A, M428L, H433K, N434A, N434F, N434S, and H435A. In embodiments, the IgG constant region comprises a M428L/N434S mutation or LS mutation. In embodiments, the IgG constant region comprises a T250Q/M428L mutation or QL mutation. In embodiments, the IgG constant region comprises an N434A mutation. In embodiments, the IgG constant region comprises a T307A/E380A/N434A mutation or AAA mutation. In embodiments, the IgG constant region comprises an 1253A/H310A/H435A mutation or IHH mutation. In embodiments, the IgG constant region comprises a H433K/N434F mutation. In embodiments, the IgG constant region comprises a M252Y/S254T/T256E and a H433K/N434F mutation in combination.

Additional exemplary mutations in the IgG constant region are described, for example, in Robbie, et al., Antimicrobial Agents and Chemotherapy (2013), 57(12):6147-6153, Dall'Acqua et al., JBC (2006), 281(33):23514-24, Dall'Acqua et al., Journal of Immunology (2002), 169:5171-80, Ko et al. Nature (2014) 514:642-645, Grevys et al. Journal of Immunology. (2015), 194(11):5497-508, and U.S. Pat. No. 7,083,784, the entire contents of which are hereby incorporated by reference.

An illustrative Fc stabilizing mutant is S228P. Illustrative Fc half-life extending mutants are T250Q, M428L, V308T, L309P, and Q311S and the present linkers may comprise 1, or 2, or 3, or 4, or 5 of these mutants.

In embodiments, the chimeric protein binds to FcRn with high affinity. In embodiments, the chimeric protein may bind to FcRn with a K_{D} of about 1 nM to about 80 nM. For example, the chimeric protein may bind to FcRn with a K_{D} of about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 71 nM, about 72 nM, about 73 nM, about 74 nM, about 75 nM, about 76 nM, about 77 nM, about 78 nM, about 79 nM, or about 80 nM. In embodiments, the chimeric protein may bind to FcRn with a K_{D} of about 9 nM. In embodiments, the chimeric protein does not substantially bind to other Fc receptors (i.e. other than FcRn) with effector function.

Without wishing to be bound by theory, including a linker comprising at least a part of an Fc domain in a chimeric protein, helps avoid formation of insoluble and, likely, non-functional protein concatamers and/or aggregates. This is in part due to the presence of cysteines in the Fc domain which are capable of forming disulfide bonds between chimeric proteins.

In embodiments, the linker is a synthetic linker such as polyethylene glycol (PEG).

In embodiments, the chimeric protein is chemically synthesized as one protein chain or each domain may be chemically synthesized separately and then combined. In embodiments, a portion of the chimeric protein is translated and a portion is chemically synthesized.

In any herein-disclosed aspect and embodiment, the chimeric protein may comprise an amino acid sequence having one or more amino acid mutations relative to any of the wild-type protein sequences for an X domain, a Y domain, and/or a Linker domain. In embodiments, the one or more amino acid mutations may be independently selected from substitutions, insertions, deletions, and truncations.

In embodiments, the amino acid mutations are amino acid substitutions, and may include conservative and/or non-conservative substitutions. “Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, lIe; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe. As used herein, “conservative substitutions” are defined as exchanges of an amino acid by another amino acid listed within the same group of the six standard amino acid groups shown above. For example, the exchange of Asp by Glu retains one negative charge in the so modified protein chain. In addition, glycine and proline may be substituted for one another based on their ability to disrupt α-helices. As used herein, “non-conservative substitutions” are defined as exchanges of an amino acid by another amino acid listed in a different group of the six standard amino acid groups (1) to (6) shown above.

In embodiments, the substitutions may also include non-classical amino acids (e.g., selenocysteine, pyrrolysine, N-formylmethionine R-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, R-alanine, fluoro-amino acids, designer amino acids such as p methyl amino acids, C α-methyl amino acids, N α-methyl amino acids, and amino acid analogs in general).

Mutations may also be made to the nucleotide sequences of the chimeric proteins by reference to the genetic code, including taking into account codon degeneracy.

### Additional Purification Steps

In embodiments, clusterin-scavenging chromatography may be first step, the last step or a step between other purification steps. Accordingly, in embodiments, clusterin-scavenging chromatography is the first chromatography step carried out during the method for isolating and/or purifying a protein comprising an Fc domain and/or a Fab domain. In embodiments, clusterin-scavenging chromatography follows one or more additional chromatography steps including but not limited to selected from an affinity chromatography, an ion exchange chromatography, a hydrophobic interaction chromatography, a reverse phase chromatography and a size exclusion chromatography. In embodiments, clusterin-scavenging chromatography follows an affinity chromatography step. In embodiments, clusterin-scavenging chromatography follows an affinity chromatography step and an ion exchange chromatography step, which may precede or follow the affinity chromatography step. In embodiments, clusterin-scavenging chromatography follows an affinity chromatography step, an ion exchange chromatography step, and a hydrophobic interaction chromatography, which may precede or follow the affinity chromatography step and the ion exchange chromatography step, or follow both the affinity chromatography step and the ion exchange chromatography step, or follow one of (and precede the other of) the affinity chromatography and the ion exchange chromatography steps.

In any of the embodiments disclosed herein, the method further comprises at least one more purification step. In embodiments, at least one purification step is liquid chromatography. In embodiments, the chromatography is an affinity chromatography. In embodiments, the affinity chromatography comprises contacting the solution comprising the protein comprising the Fc domain and/or the Fab domain with a moiety having affinity for the X, the Y, the Fc domain and/or the Fab domain. In embodiments, the affinity chromatography precedes the steps using the polypeptide capable of binding clusterin of any embodiments disclosed herein.

In embodiments, the moiety having affinity for the X and/or Y is an antibody, or a binding fragment thereof, or an antibody-like molecule or a binding fragment thereof. In embodiments, the antibody-like molecule is selected from an antibody, a single-domain antibody, a heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂, or a binding fragment thereof. These types of binding agents are disclosed In U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317

In embodiments, the immobilized moiety comprises a fragment of the native binding partner for the X domain or the Y domain. In embodiments, the fragment has a binding affinity for the X domain or the Y domain that is substantially equivalent to the binding affinity that the native binding partner has for the X domain or the Y domain; the fragment has a binding affinity for the X domain or the Y domain that is reduced relative to the binding affinity that the native binding partner has for the X domain or the Y domain; or the fragment has a binding affinity for the X domain or the Y domain that is increased relative to the binding affinity that the native binding partner has for the X domain or the Y domain. In embodiments, the fragment comprises at least one mutation relative to the native binding partner for the X domain or the Y domain; in embodiments, the at least one mutation of the fragment provides a different binding affinity relative to the binding affinity of a fragment lacking the at least one mutation. In embodiments, a binding affinity of the X domain or the Y domain for the immobilized moiety is about 100 nM or less.

As examples, in embodiments, a binding affinity of the X domain or the Y domain for the immobilized moiety is less than 100 nM, is less than 90 nM, is less than 80 nM, is less than 70 nM, is less than 60 nM, is less than 50 nM, is less than 40 nM, is less than 30 nM, is less than 20 nM, is less than 10 nM, is less than 5 nM, is less than 1 nM, is less than 900 pM, is less than 800 pM, is less than 700 pM, is less than 600 pM, is less than 500 pM, is less than 400 pM, is less than 300 pM, is less than 200 pM, is less than 100 pM, is less than 90 pM, is less than 80 pM, is less than 70 pM, is less than 60 pM, is less than 50 pM, is less than 40 pM, is less than 30 pM, is less than 20 pM, is less than 10 pM, is less than 5 pM, or is less than 1 pM.

In embodiments, the moiety having affinity for the Fab domain is an antibody, or a binding fragment thereof, or an antibody-like molecule or a binding fragment thereof. In embodiments, the antibody-like molecule is selected from an antibody, a single-domain antibody, a heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂, or a binding fragment thereof. These types of binding agents are disclosed In U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317

In embodiments, the moiety having affinity for the Fc domain is an antibody, or a binding fragment thereof, or an antibody-like molecule or a binding fragment thereof. In embodiments, the antibody-like molecule is selected from an antibody, a single-domain antibody, a heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂, or a Fc domain binding fragment thereof. In embodiments, the moiety having affinity for the Fc domain is selected from a protein A, a protein G, protein L, protein M, or a derivative thereof. These types of binding agents are disclosed In U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317.

In embodiments, the affinity chromatography is selected from FcXL chromatography, protein A chromatography, a protein G chromatography, protein L chromatography, and protein M chromatography.

In embodiments, the affinity chromatography comprises: contacting a solution comprising the protein comprising the Fc domain and/or the Fab domain with the moiety having affinity for the X, the Y, the Fc domain and/or the Fab domain that is conjugated to a solid support, and thereby directly or indirectly attaching the protein comprising the Fc domain and/or the Fab domain and/or at least one HCP to the solid support. In embodiments, the solid support is a bead or a chromatography resin. In embodiments, the bead is an agarose bead. In embodiments, the agarose bead is an aldehyde-activated agarose bead. In embodiments, the immobilized moiety is coupled to the bead via a free NH₂. In embodiments, the chromatography resin comprises crosslinked poly[styrene divinylbenzene].

In embodiments, the solution comprising the protein comprising the Fc domain and/or the Fab domain is selected from an eluate from a chromatography step, a flow through sample of a chromatography step, a culture supernatant, and a cell-free extract. In embodiments, the eluate from the chromatography step is selected from an eluate from an ion exchange chromatography, a hydrophobic interaction chromatography, a reverse phase chromatography and a size exclusion chromatography. In embodiments, the flow through sample of the chromatography step is a flow through from a clusterin scavenging chromatography. In embodiments, the clusterin is scavenged using a polypeptide immobilized onto the surface of a solid support, wherein the immobilized polypeptide is capable of binding clusterin. In embodiments, the polypeptide capable of binding clusterin is selected from an antibody, a single-domain antibody, a heavy-chain-only antibody (VHH), a single-chain antibody (scFv), or a binding fragment thereof.

In embodiments, the moiety having affinity for the X, the Y, the Fc domain and/or the Fab domain that is conjugated to the solid support is located in a second chromatography column. In embodiments, the second chromatography column is washed with a buffer. In embodiments, the buffer comprises an ingredient selected from a salt, a detergent, an alcohol, a buffering agent and a combination thereof. In embodiments, the buffer has a temperature between 0° C. and 4° C. In embodiments, the wash with the buffer removes the protein comprising the Fc domain and/or the Fab domain and/or the at least one HCP that is indirectly attached to the solid support.

In embodiments, the method further comprises at least one more purification step. In embodiments, at least one purification step is liquid chromatography. In embodiments, the chromatography is an affinity chromatography. In embodiments, the affinity chromatography comprises contacting the solution comprising the chimeric protein with a moiety having affinity for the X domain, the Y domain and/or the Fc domain.

In embodiments, the moiety having affinity for the X domain is an antibody, or a binding fragment thereof, or an antibody-like molecule or a binding fragment thereof. In embodiments, the antibody-like molecule is selected from an antibody, a single-domain antibody, a heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂, or a binding fragment thereof.

In embodiments, the moiety having affinity for the Y domain is an antibody, or a binding fragment thereof, or an antibody-like molecule or a binding fragment thereof. In embodiments, the antibody-like molecule is selected from an antibody, a single-domain antibody, a heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂, or a binding fragment thereof. In embodiments, the moiety having affinity for the Fc domain is an antibody, or a binding fragment thereof, or an antibody-like molecule or a binding fragment thereof.

In embodiments, the antibody-like molecule is selected from an antibody, a single-domain antibody, a heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂, or a Fc domain binding fragment thereof.

In embodiments, the moiety having affinity for the Fc domain is selected from a protein A, a protein G, protein L, protein M, or a derivative thereof. In embodiments, the affinity chromatography is selected from FcXL chromatography, protein A chromatography, a protein G chromatography, protein L chromatography, and protein M chromatography. In embodiments, the affinity chromatography comprises: contacting a solution comprising the chimeric protein with the moiety having affinity for the X domain, the Y domain and/or the Fc domain that is conjugated to a solid support, and thereby directly or indirectly attaching the chimeric protein and/or at least one HCP to the solid support. In embodiments, the solid support is a bead or a chromatography resin. In embodiments, the bead is an agarose bead. In embodiments, the agarose bead is an aldehyde-activated agarose bead. In embodiments, the immobilized moiety is coupled to the bead via a free NH₂. In embodiments, the chromatography resin comprises crosslinked poly[styrene divinylbenzene].

In embodiments, the solution comprising the chimeric protein is selected from an eluate from a chromatography step, a flow through sample of a chromatography step, a culture supernatant, and a cell-free extract. In embodiments, the eluate from the chromatography step is selected from an eluate from an ion exchange chromatography, a hydrophobic interaction chromatography, a reverse phase chromatography and a size exclusion chromatography. In embodiments, the flow through sample of the chromatography step is a flow through from a clusterin scavenging chromatography. In embodiments, the clusterin is scavenged using a polypeptide immobilized onto the surface of a solid support, wherein the immobilized polypeptide is capable of binding clusterin. In embodiments, the polypeptide capable of binding clusterin is selected from an antibody, a single-domain antibody, a heavy-chain-only antibody (VHH), a single-chain antibody (scFv), or a binding fragment thereof.

In embodiments, the moiety having affinity for the X domain, the Y domain and/or the Fc domain that is conjugated to the solid support is located in a second chromatography column. In embodiments, the second chromatography column is washed with a buffer. In embodiments, the wash with the buffer removes the chimeric protein and/or the at least one HCP that is indirectly attached to the solid support.

In embodiments, the method further comprises contacting the chimeric protein that is directly attached to the solid support with a first elution solution that releases the X domain, the Fc domain, and/or the Y domain from the second chromatography column, thereby forming a first eluate which comprises the chimeric protein. In embodiments, the affinity chromatography removes at least one HCP. In embodiments, the first eluate comprises fewer contaminants than the solution comprising the chimeric protein that was contacted with the moiety having affinity for the X domain, the Y domain and/or the Fc domain that is conjugated to the solid support. In embodiments, the contaminants comprise components of a mammalian cell that harbors a nucleic acid that expresses the chimeric protein.

In embodiments, the method further comprises at least one more purification step. In embodiments, at least one purification step is liquid chromatography. In embodiments, the chromatography is an ion exchange chromatography. In embodiments, the chromatography is an anion exchange chromatography or a cation exchange chromatography. In embodiments, the ion exchange chromatography comprises: contacting a solution comprising the chimeric protein with an ion exchange matrix that is conjugated to a second solid support, thereby directly or indirectly attaching the chimeric protein and/or at least one HCP to the second solid support. In embodiments, the ion exchange matrix is selected from a weak carboxymethyl cation-exchanger, a strong sulfopropyl (SP) exchanger, and a weak diethylaminoethyl anion exchanger, and a strong quaternary aminoethyl (QAE) exchanger. In embodiments, the solid support is a bead or a chromatography resin. In embodiments, the bead is an agarose bead. In embodiments, the agarose bead is an aldehyde-activated agarose bead. In embodiments, the immobilized moiety is coupled to the bead via a free NH₂. In embodiments, the chromatography resin comprises crosslinked poly[styrene divinylbenzene].

In embodiments, the solution comprising the chimeric protein is selected from the first eluate, an eluate from a chromatography step, a flow through sample of a chromatography step, a culture supernatant, and a cell-free extract. In embodiments, the eluate from the chromatography step is selected from an eluate from a hydrophobic interaction chromatography, a reverse phase chromatography and a size exclusion chromatography. In embodiments, the flow through sample of the chromatography step is a flow through from a clusterin scavenging chromatography. In embodiments, the clusterin is scavenged using a polypeptide immobilized onto the surface of a solid support, wherein the immobilized polypeptide is capable of binding clusterin. In embodiments, the polypeptide capable of binding clusterin is selected from an antibody, a single-domain antibody, a heavy-chain-only antibody (VHH), a single-chain antibody (scFv), or a binding fragment thereof.

In embodiments, the ion exchange matrix that is conjugated to the second solid support is located in a third chromatography column. In embodiments, the third chromatography column is washed with a buffer. In embodiments, the washing step the third chromatography column removes the chimeric protein and/or the at least one HCP that is indirectly attached to the second solid support.

In embodiments, the method further comprises contacting the chimeric protein that is directly attached to the second solid support with a first elution solution that releases the chimeric protein from the second chromatography column, thereby forming a second eluate which comprises the chimeric protein. In embodiments, the ion exchange chromatography removes at least one HCP. In embodiments, the second eluate comprises fewer contaminants than the solution comprising the chimeric protein that was contacted with the ion exchange matrix that is conjugated to a second solid support. In embodiments, the contaminants comprise components of a mammalian cell that harbors a nucleic acid that expresses the chimeric protein.

In embodiments, the method further comprises at least one more purification step. In embodiments, at least one purification step is liquid chromatography. In embodiments, the chromatography is a hydrophobic interaction chromatography. In embodiments, the hydrophobic interaction chromatography comprises: contacting a solution comprising the chimeric protein with a hydrophobic interaction chromatography (HIC) media that is conjugated to a third solid support, thereby directly or indirectly attaching the chimeric protein and/or at least one HCP to the third solid support. In embodiments, the HIC media is selected from MACRO-PREP METHYL, MACRO-PREP T-BUTYL (BIO-RAD), CAPTO PHENYL, CAPTO BUTYL (GE Healthcare), TOYOPEARL HIC (Tosoh), FRACTOGEL EMD PHENYL (Merck Millipore) and a membrane adsorber (e.g., SARTOBIND HIC (Sartorius)). In embodiments, the solid support is a bead, a chromatography resin or a membrane. In embodiments, the bead is an agarose bead. In embodiments, the agarose bead is an aldehyde-activated agarose bead. In embodiments, the immobilized media is coupled to the bead via a free NH₂. In embodiments, the chromatography resin comprises crosslinked poly[styrene divinylbenzene].

In embodiments, the solution comprising the chimeric protein is selected from the first eluate, the second eluate, an eluate from a chromatography step, a flow through sample of a chromatography step, a culture supernatant, and a cell-free extract. In embodiments, the eluate from the chromatography step is selected from an eluate from a reverse phase chromatography and a size exclusion chromatography. In embodiments, the flow through sample of the chromatography step is a flow through from a clusterin scavenging chromatography. In embodiments, the clusterin is scavenged using a polypeptide immobilized onto the surface of a solid support, wherein the immobilized polypeptide is capable of binding clusterin. In embodiments, the polypeptide capable of binding clusterin is selected from an antibody, a single-domain antibody, a heavy-chain-only antibody (VHH), a single-chain antibody (scFv), or a binding fragment thereof.

In embodiments, the HIC media that is conjugated to the third solid support is located in a fourth chromatography column or a membrane. In embodiments, the fourth chromatography column or the membrane is washed with a buffer. In embodiments, the washing step the fourth chromatography column removes the chimeric protein and/or the at least one HCP that is indirectly attached to the third solid support.

In embodiments, the method further comprises contacting the chimeric protein that is directly attached to the third solid support with a first elution solution that releases the chimeric protein from the third chromatography column, thereby forming a third eluate which comprises the chimeric protein. In embodiments, the hydrophobic interaction chromatography removes at least one HCP. In embodiments, the third eluate comprises fewer contaminants than the solution comprising the chimeric protein that was contacted with the HIC media that is conjugated to a third solid support. In embodiments, the contaminants comprise components of a mammalian cell that harbors a nucleic acid that expresses the chimeric protein.

In embodiments, the method further comprises contacting the protein comprising the Fc domain and/or the Fab domain that is directly attached to the solid support with a first elution solution that releases the X, the Y, the Fc domain, and/or the Fab domain from the second chromatography column, thereby forming a first eluate which comprises the protein comprising the Fc domain and/or the Fab domain. In embodiments, the affinity chromatography removes at least one HCP. In embodiments, the first eluate comprises fewer contaminants than the solution comprising the protein comprising the Fc domain and/or the Fab domain that was contacted with the moiety having affinity for the X, the Y, the Fc domain and/or the Fab domain that is conjugated to the solid support. In embodiments, the contaminants comprise components of a mammalian cell that harbors a nucleic acid that expresses the protein comprising the Fc domain and/or the Fab domain.

In embodiments, the method further comprises at least one more purification step. In embodiments, at least one purification step is liquid chromatography. In embodiments, the chromatography is an ion exchange chromatography. In embodiments, the chromatography is an anion exchange chromatography. In embodiments, the chromatography is a cation exchange chromatography. In embodiments, the ion exchange chromatography comprises: contacting a solution comprising the protein comprising the Fc domain and/or the Fab domain with an ion exchange matrix that is conjugated to a second solid support, thereby directly or indirectly attaching the protein comprising the Fc domain and/or the Fab domain and/or at least one HCP to the second solid support. In embodiments, the solid support is a bead or a chromatography resin. In embodiments, the bead is an agarose bead. In embodiments, the agarose bead is an aldehyde-activated agarose bead. In embodiments, the immobilized matrix is coupled to the bead via a free NH₂.

In embodiments, the solution comprising the protein comprising the Fc domain and/or the Fab domain is selected from the first eluate, an eluate from a chromatography step, a flow through sample of a chromatography step, a culture supernatant, and a cell-free extract. In embodiments, the eluate from the chromatography step is selected from an eluate from a hydrophobic interaction chromatography, a reverse phase chromatography and a size exclusion chromatography. In embodiments, the flow through sample of the chromatography step is a flow through from a clusterin scavenging chromatography. In embodiments, the clusterin is scavenged using a polypeptide immobilized onto the surface of a solid support, wherein the immobilized polypeptide is capable of binding clusterin. In embodiments, the polypeptide capable of binding clusterin is selected from an antibody, a single-domain antibody, a heavy-chain-only antibody (VHH), a single-chain antibody (scFv), or a binding fragment thereof.

In embodiments, the chromatography resin comprises crosslinked poly[styrene divinylbenzene]. In embodiments, the ion exchange matrix is selected from a weak carboxymethyl cation-exchanger, a strong sulfopropyl (SP) exchanger, and a weak diethylaminoethyl anion exchanger, and a strong quaternary aminoethyl (QAE) exchanger. In embodiments, the ion exchange matrix that is conjugated to the second solid support is located in a third chromatography column. In embodiments, the third chromatography column is washed with a buffer. In embodiments, the buffer comprises an ingredient selected from a salt, a detergent, an alcohol, a buffering agent and a combination thereof. In embodiments, the buffer has a temperature between 0° C. and 4° C. In embodiments, the washing step the third chromatography column removes the protein comprising the Fc domain and/or the Fab domain and/or the at least one HCP that is indirectly attached to the second solid support. In embodiments, the method further comprises contacting the protein comprising the Fc domain and/or the Fab domain that is directly attached to the second solid support with a first elution solution that releases the protein comprising the Fc domain and/or the Fab domain from the second chromatography column, thereby forming a second eluate which comprises the protein comprising the Fc domain and/or the Fab domain. In embodiments, the ion exchange chromatography removes at least one HCP. In embodiments, the second eluate comprises fewer contaminants than the solution comprising the protein comprising the Fc domain and/or the Fab domain that was contacted with the ion exchange matrix that is conjugated to a second solid support. In embodiments, the contaminants comprise components of a mammalian cell that harbors a nucleic acid that expresses the protein comprising the Fc domain and/or the Fab domain.

In embodiments, the method further comprises at least one more purification step. In embodiments, at least one purification step is liquid chromatography. In embodiments, the chromatography is a hydrophobic interaction chromatography. In embodiments, the hydrophobic interaction chromatography comprises: contacting a solution comprising the protein comprising the Fc domain and/or the Fab domain with a hydrophobic interaction chromatography (HIC) media that is conjugated to a third solid support, thereby directly or indirectly attaching the protein comprising the Fc domain and/or the Fab domain and/or at least one HCP to the third solid support. In embodiments, the solid support is a bead, a chromatography resin or a membrane. In embodiments, the bead is an agarose bead. In embodiments, the agarose bead is an aldehyde-activated agarose bead. In embodiments, the immobilized matrix is coupled to the bead via a free NH₂. In embodiments, the chromatography resin comprises crosslinked poly[styrene divinylbenzene].

In embodiments, the solution comprising the protein comprising the Fc domain and/or the Fab domain is selected from the first eluate, the second eluate, an eluate from a chromatography step, a flow through sample of a chromatography step, a culture supernatant, and a cell-free extract. In embodiments, the eluate from the chromatography step is selected from an eluate from a reverse phase chromatography and a size exclusion chromatography. In embodiments, the flow through sample of the chromatography step is a flow through from a clusterin scavenging chromatography. In embodiments, the clusterin is scavenged using a polypeptide immobilized onto the surface of a solid support, wherein the immobilized polypeptide is capable of binding clusterin. In embodiments, the polypeptide capable of binding clusterin is selected from an antibody, a single-domain antibody, a heavy-chain-only antibody (VHH), a single-chain antibody (scFv), or a binding fragment thereof.

In embodiments, the HIC media is selected from MACRO-PREP METHYL, MACRO-PREP T-BUTYL (BIO-RAD), CAPTO PHENYL, CAPTO BUTYL (GE Healthcare), TOYOPEARL HIC (Tosoh), FRACTOGEL EMD PHENYL (Merck Millipore) and a membrane adsorber (e.g. SARTOBIND HIC (Sartorius)). In embodiments, the HIC media that is conjugated to the third solid support is located in a fourth chromatography column or a membrane. In embodiments, the fourth chromatography column or the membrane is washed with a buffer. In embodiments, the buffer comprises an ingredient selected from a salt, a detergent, an alcohol, a buffering agent and a combination thereof. In embodiments, the buffer has a temperature between 0° C. and 4° C. In embodiments, the washing step the fourth chromatography column removes the protein comprising the Fc domain and/or the Fab domain and/or the at least one HCP that is indirectly attached to the third solid support.

In embodiments, the method further comprises contacting the protein comprising the Fc domain and/or the Fab domain that is directly attached to the third solid support with a first elution solution that releases the protein comprising the Fc domain and/or the Fab domain from the third chromatography column, thereby forming a third eluate which comprises the protein comprising the Fc domain and/or the Fab domain. In embodiments, the hydrophobic interaction chromatography removes at least one HCP. In embodiments, the third eluate comprises fewer contaminants than the solution comprising the protein comprising the Fc domain and/or the Fab domain that was contacted with the HIC media that is conjugated to a third solid support. In embodiments, the contaminants comprise components of a mammalian cell that harbors a nucleic acid that expresses the protein comprising the Fc domain and/or the Fab domain.

### Illustrative Purification Schemes

In aspects, the purification scheme for purifying a protein comprising an Fc domain and/or a Fab domain comprises multiple steps. In embodiments, the purification scheme for purifying a protein comprising an Fc domain and/or a Fab domain comprises at least one, or at least two or at least three or at least four, or at least five or at least six steps. In embodiments, the purification scheme for purifying a protein comprising an Fc domain and/or a Fab domain comprises a clusterin-scavenging chromatography step. In embodiments, the purification scheme for purifying a protein comprising an Fc domain and/or a Fab domain comprises a clusterin-scavenging chromatography step, and an affinity chromatography step. In embodiments, the purification scheme for purifying a protein comprising an Fc domain and/or a Fab domain comprises a clusterin-scavenging chromatography step, and an ion exchange chromatography step. In embodiments, the purification scheme for purifying a protein comprising an Fc domain and/or a Fab domain comprises a clusterin-scavenging chromatography step, and a hydrophobic interaction chromatography step. In embodiments, the purification scheme for purifying a protein comprising an Fc domain and/or a Fab domain comprises a clusterin-scavenging chromatography step, an affinity chromatography step, and an ion exchange chromatography step. In embodiments, the purification scheme for purifying a protein comprising an Fc domain and/or a Fab domain comprises a clusterin-scavenging chromatography step, an affinity chromatography step, and an ion exchange chromatography step. In embodiments, the purification scheme for purifying a protein comprising an Fc domain and/or a Fab domain comprises a clusterin-scavenging chromatography step, an affinity chromatography step, and hydrophobic interaction chromatography step. In embodiments, the purification scheme for purifying a protein comprising an Fc domain and/or a Fab domain comprises a clusterin-scavenging chromatography step, a hydrophobic interaction chromatography step and an ion exchange chromatography step. In embodiments, the purification scheme for purifying a protein comprising an Fc domain and/or a Fab domain comprises a clusterin-scavenging chromatography step, an affinity chromatography step, a hydrophobic interaction chromatography step and an ion exchange chromatography step. These steps may follow in any order, optionally interspersed with additional purification steps. The purification scheme of any embodiment may be used for isolating and/or purifying a protein comprising an Fc domain and/or a Fab domain of any embodiment disclosed herein.

In one aspect, the present disclosure relates to a method for isolating and/or purifying a protein comprising an Fc domain and/or a Fab domain, the method comprising: (i) a clusterin-scavenging chromatography, (ii) an affinity chromatography using a moiety immobilized on to a solid support, the moiety having affinity for the X, the Y, the Fc domain and/or the Fab domain, (iii) an ion exchange chromatography, and/or (iv) a hydrophobic interaction chromatography, and thereby isolating and/or purifying the protein comprising the Fc domain and/or the Fab domain. In embodiments, the clusterin-scavenging chromatography is performed using the methods of any of the embodiments disclosed herein. In embodiments, the moiety having affinity for the X, the Y, the Fc domain and/or the Fab domain of any of the embodiments disclosed herein may be used. In embodiments, the affinity chromatography is selected from FcXL chromatography, protein A chromatography, a protein G chromatography, protein L chromatography, and protein M chromatography. In embodiments, the ion exchange chromatography is selected from cation exchange chromatography and anion exchange chromatography. In embodiments, the ion exchange chromatography is selected from a weak carboxymethyl cation-exchanger chromatography, a strong sulfopropyl (SP) exchanger chromatography, and a weak diethylaminoethyl anion exchanger chromatography, and a strong quaternary aminoethyl (QAE) exchanger chromatography. In embodiments, the ion exchange chromatography is performed using the methods of any of the embodiments disclosed herein. In embodiments, the hydrophobic interaction chromatography is selected from MACRO-PREP METHYL chromatography, MACRO-PREP T-BUTYL (BIO-RAD) chromatography, CAPTO PHENYL chromatography, CAPTO BUTYL (GE Healthcare) chromatography, TOYOPEARL HIC (Tosoh) chromatography, FRACTOGEL EMD PHENYL (Merck Millipore) chromatography, and a membrane adsorber (e.g. SARTOBIND HIC (Sartorius)) chromatography. In embodiments, the hydrophobic interaction chromatography is performed using the methods of any of the embodiments disclosed herein.

In embodiments, the clusterin-scavenging chromatography follows one or more of the affinity chromatography, ion exchange chromatography and hydrophobic interaction chromatography. In embodiments, the clusterin-scavenging chromatography follows the affinity chromatography step. In embodiments, clusterin-scavenging chromatography follows the affinity chromatography step and the ion exchange chromatography step. In embodiments, clusterin-scavenging chromatography follows the affinity chromatography step, the ion exchange chromatography step, and the hydrophobic interaction chromatography step. In embodiments, the clusterin-scavenging chromatography precedes the affinity chromatography step. In embodiments, clusterin-scavenging chromatography precedes the affinity chromatography step and the ion exchange chromatography step. In embodiments, clusterin-scavenging chromatography precedes the affinity chromatography step, the ion exchange chromatography step, and the hydrophobic interaction chromatography step. In embodiments, the affinity chromatography follows the ion exchange chromatography and/or the hydrophobic interaction chromatography. In embodiments, the ion exchange chromatography follows the affinity chromatography and/or the hydrophobic interaction chromatography. In embodiments, the hydrophobic interaction chromatography follows the ion exchange chromatography and/or the affinity chromatography. In embodiments, the method comprises one or more further purification steps. In embodiments, the further purification steps comprise a reverse phase chromatography and/or a size exclusion chromatography. In embodiments, the hydrophobic interaction chromatography follows the ion exchange chromatography and/or the affinity chromatography. In embodiments, the method comprises one or more further purification steps. In embodiments, the further purification steps comprise a reverse phase chromatography and/or a size exclusion chromatography.

In one aspect, the present disclosure relates to a method for isolating and/or purifying a protein comprising an Fc domain and/or a Fab domain, the method comprising: (i) a clusterin-scavenging chromatography comprising (a) providing a solution comprising the protein comprising the Fc domain and/or the Fab domain, the solution further comprising one or more host cell proteins (HCP), (b) contacting the solution comprising the protein comprising the Fc domain and/or the Fab domain with a polypeptide immobilized onto the surface of a solid support, wherein the immobilized polypeptide is capable of binding clusterin, and (c) recovering the protein that does not bind to the polypeptide immobilized onto the surface of the solid support, (ii) an affinity chromatography selected from FcXL chromatography, protein A chromatography, a protein G chromatography, protein L chromatography, and protein M chromatography, (iii) an ion exchange chromatography selected from cation exchange chromatography and anion exchange chromatography, and/or (iv) a hydrophobic interaction chromatography and thereby isolating and/or purifying the protein comprising the Fc domain and/or the Fab domain.

In embodiments, the clusterin-scavenging chromatography follows one or more of the affinity chromatography, ion exchange chromatography and hydrophobic interaction chromatography. In embodiments, the clusterin-scavenging chromatography follows the affinity chromatography step. In embodiments, clusterin-scavenging chromatography follows the affinity chromatography step and the ion exchange chromatography step. In embodiments, clusterin-scavenging chromatography follows the affinity chromatography step, the ion exchange chromatography step, and the hydrophobic interaction chromatography step. In embodiments, the clusterin-scavenging chromatography precedes the affinity chromatography step. In embodiments, clusterin-scavenging chromatography precedes the affinity chromatography step and the ion exchange chromatography step. In embodiments, clusterin-scavenging chromatography precedes the affinity chromatography step, the ion exchange chromatography step, and the hydrophobic interaction chromatography step. In embodiments, the affinity chromatography follows the ion exchange chromatography and/or the hydrophobic interaction chromatography. In embodiments, the ion exchange chromatography follows the affinity chromatography and/or the hydrophobic interaction chromatography. In embodiments, the hydrophobic interaction chromatography follows the ion exchange chromatography and/or the affinity chromatography. In embodiments, the method comprises one or more further purification steps. In embodiments, the further purification steps comprise a reverse phase chromatography and/or a size exclusion chromatography.

In embodiments, the step (c) removes at least one HCP. In embodiments, the at least one HCP comprises free clusterin, clusterin bound to the polypeptide, and/or any additional HCPs contemporaneously bound to clusterin and/or the polypeptide. In embodiments, step (c) removes clusterin and at least one more HCP. In embodiments, the clusterin is Cricetulus griseus (Chinese hamster) clusterin. In embodiments, the at least one HCP further comprises a protein that directly or indirectly binds clusterin. In embodiments, the polypeptide capable of binding clusterin is directly immobilized to a solid support and the step (c) comprises: (i) passing the solution across the polypeptide immobilized onto the surface of the solid support using buffer conditions that facilitate binding of clusterin to the immobilized polypeptide, and (ii) recovering a protein-containing solution that has passed across the immobilized polypeptide, wherein the recovered protein-containing solution is substantially free of clusterin.

In one aspect, the present disclosure relates to a method for isolating and/or purifying a protein comprising an Fc domain and/or a Fab domain, the method comprises (i) providing a solution comprising the protein comprising the Fc domain and/or the Fab domain, and performing: (a) a clusterin-scavenging chromatography, (b) an affinity chromatography, (c) an ion exchange chromatography, and/or (d) a hydrophobic interaction chromatography, and thereby isolating and/or purifying the protein comprising the Fc domain and/or the Fab domain.

In embodiments, the clusterin-scavenging chromatography comprises: (a) providing a solution comprising the protein comprising the Fc domain and/or the Fab domain, the solution further comprising one or more host cell proteins (HCP), (b) contacting the solution comprising the protein comprising the Fc domain and/or the Fab domain with a polypeptide immobilized onto the surface of a solid support, wherein the immobilized polypeptide is capable of binding clusterin, and (c) recovering the protein that does not bind to the polypeptide immobilized onto the surface of the solid support. In embodiments, the affinity chromatography uses a moiety having affinity for the X, the Y, the Fc domain and/or the Fab domain. In embodiments, the moiety having affinity for the Fc domain is selected from protein A, a protein G, protein L, and protein M. In embodiments, the ion exchange chromatography is selected from cation exchange chromatography and anion exchange chromatography

In one aspect, the present disclosure relates to a method for isolating and/or purifying a protein comprising an Fc domain and/or a Fab domain, the method comprising: (i) performing a clusterin-scavenging chromatography comprising: (a) providing a solution comprising the protein comprising the Fc domain and/or the Fab domain, the solution further comprising one or more host cell proteins (HCP), (b) contacting the solution comprising the protein comprising the Fc domain and/or the Fab domain with a polypeptide immobilized onto the surface of a solid support, wherein the immobilized polypeptide is capable of binding clusterin, and (c) recovering the protein that does not bind to the polypeptide immobilized onto the surface of the solid support, (ii) performing an affinity chromatography selected from FcXL chromatography, protein A chromatography, a protein G chromatography, protein L chromatography, and protein M chromatography, (iii) performing an ion exchange chromatography selected from cation exchange chromatography and anion exchange chromatography, and/or (iv) performing a hydrophobic interaction chromatography, and thereby isolating and/or purifying the protein comprising the Fc domain and/or the Fab domain.

In embodiments, the clusterin-scavenging chromatography follows one or more of the affinity chromatography, ion exchange chromatography and hydrophobic interaction chromatography. In embodiments, the clusterin-scavenging chromatography precedes one or more of the affinity chromatography, ion exchange chromatography and hydrophobic interaction chromatography.

In embodiments, the method removes clusterin and at least one more HCP. In embodiments, the at least one more HCP is selected from glyceraldehyde-3-phosphate dehydrogenase (GAPDH), actin (e.g., actin, cytoplasmic 2 isoform X2), and serine protease HTRA1 isoform X2 (HTRA).

In embodiments, the method removes at least by 30%, or at least 40%, or at least 40%, or at least 50%, or at least 60%, or at least 70%, or at least 80%, or at least 90% clusterin compared to the solution comprising the protein comprising the Fc domain and/or the Fab domain. In embodiments, the method removes at least by 10%, or at least 20%, or at least 30%, or at least 40%, or at least 50%, or at least 60%, or at least 70% of the at least one more HCP compared to the solution comprising the protein comprising the Fc domain and/or the Fab domain. In embodiments, the clusterin is Cricetulus griseus (Chinese hamster) clusterin. In embodiments, the at least one more HCP is at least one more Cricetulus griseus (Chinese hamster) HCP.

In embodiments, the ion exchange chromatography is selected from a weak carboxymethyl cation-exchanger chromatography, a strong sulfopropyl (SP) exchanger chromatography, and a weak diethylaminoethyl anion exchanger chromatography, and a strong quaternary aminoethyl (QAE) exchanger chromatography. In embodiments, the hydrophobic interaction chromatography is selected from MACRO-PREP METHYL chromatography, MACRO-PREP T-BUTYL (BIO-RAD) chromatography, CAPTO PHENYL chromatography, CAPTO BUTYL (GE Healthcare) chromatography, TOYOPEARL HIC (Tosoh) chromatography, FRACTOGEL EMD PHENYL (Merck Millipore) chromatography, and a membrane adsorber (e.g. SARTOBIND HIC (Sartorius)) chromatography.

In embodiments, the clusterin-scavenging chromatography is performed using the methods of any of the embodiments disclosed herein. In embodiments, the affinity chromatography is performed using the methods of any of the embodiments disclosed herein. In embodiments, the ion exchange chromatography is performed using the methods of any of the embodiments disclosed herein. In embodiments, the hydrophobic interaction chromatography is performed using the methods of any of the embodiments disclosed herein.

### Pharmaceutical Compositions of the Proteins Purified According to the Methods Disclosed Herein

In one aspect, the present disclosure relates to an isolated and/or purified chimeric protein prepared using the method of any of the embodiments disclosed herein.

Aspects of the present invention include a pharmaceutical composition comprising a therapeutically effective amount of a protein comprising an Fc domain and/or a Fab domain (e.g. antibodies, Fab-fusion proteins and Fc-fusion proteins) as manufactured by a method disclosed herein.

The protein comprising the Fc domain and/or the Fab domain (e.g. an antibody, a Fab-fusion protein or a Fc-fusion proteins) as manufactured by a method disclosed herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

In embodiments, the compositions disclosed herein are in the form of a pharmaceutically acceptable salt.

Further, any protein comprising the Fc domain and/or the Fab domain (e.g. an antibody, a Fab-fusion protein or a Fc-fusion proteins) as manufactured by a method disclosed herein for administration to a subject as a component of a composition, e.g., pharmaceutical composition that comprises a pharmaceutically acceptable carrier or vehicle. Such pharmaceutical compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent disclosed herein, if desired, can also comprise minor amounts of wetting or emulsifying agents, or pH buffering agents.

In embodiments, the compositions, e.g., pharmaceutical compositions, disclosed herein are resuspended in a saline buffer (including, without limitation TBS, PBS, and the like).

In embodiments, the protein comprising the Fc domain and/or the Fab domain (e.g. an antibody, a Fab-fusion protein or a Fc-fusion proteins) as manufactured by a method disclosed herein may by conjugated and/or fused with another agent to extend half-life or otherwise improve pharmacodynamic and pharmacokinetic properties. In embodiments, the proteins comprising the Fc domain and/or the Fab domain (e.g. an antibody, a Fab-fusion protein or a Fc-fusion proteins) may be fused or conjugated with one or more of PEG, XTEN (e.g., as rPEG), polysialic acid (POLYXEN), albumin (e.g., human serum albumin or HAS), elastin-like protein (ELP), PAS, HAP, GLK, CTP, transferrin, and the like. In embodiments, each of the individual protein comprising the Fc domain and/or the Fab domain (e.g. an antibody, a Fab-fusion protein or a Fc-fusion proteins) is fused to one or more of the agents described in BioDrugs (2015) 29:215-239, the entire contents of which are hereby incorporated by reference.

The present invention includes the proteins comprising the Fc domain and/or the Fab domain (e.g. an antibody, a Fab-fusion protein or a Fc-fusion proteins) as manufactured by a method disclosed herein in various formulations of pharmaceutical composition. Any protein comprising the Fc domain and/or the Fab domain (e.g. an antibody, a Fab-fusion protein or a Fc-fusion proteins) as manufactured by a method disclosed herein can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. DNA or RNA constructs encoding the protein sequences may also be used. In embodiments, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

Where necessary, the pharmaceutical compositions comprising the protein comprising the Fc domain and/or the Fab domain (e.g. an antibody, a Fab-fusion protein or a Fc-fusion proteins) as manufactured by a method disclosed herein can also include a solubilizing agent. Also, the agents can be delivered with a suitable vehicle or delivery device as known in the art.

The pharmaceutical compositions comprising the protein comprising the Fc domain and/or the Fab domain (e.g. an antibody, a Fab-fusion protein or a Fc-fusion proteins) as manufactured by a method disclosed herein may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In embodiments, any protein comprising the Fc domain and/or the Fab domain (e.g. an antibody, a Fab-fusion protein or a Fc-fusion proteins) as manufactured by a method disclosed herein is formulated in accordance with routine procedures as a pharmaceutical composition adapted for a mode of administration disclosed herein.

### Cells and Nucleic Acids

In embodiments, the protein comprising the Fc domain and/or the Fab domain (e.g. an antibody, a Fab-fusion protein or a Fc-fusion proteins) is translated as a single unit in a prokaryotic cell, a eukaryotic cell, or a cell-free expression system.

In embodiments, the protein comprising the Fc domain and/or the Fab domain (e.g. an antibody, a Fab-fusion protein or a Fc-fusion proteins) is producible in a mammalian host cell as a secretable and fully functional single protein chain.

In embodiments, protein comprising the Fc domain and/or the Fab domain (e.g. an antibody, a Fab-fusion protein or a Fc-fusion proteins) refers to a recombinant protein of multiple protein chains, e.g., multiple extracellular domains disclosed herein, that are combined (via covalent or no-covalent bonding) to yield a single unit, e.g., in vitro (e.g., with one or more synthetic linkers disclosed herein).

Aspects of the present invention provide an expression vector comprising a nucleic acid which encodes a protein comprising the Fc domain and/or the Fab domain (e.g. an antibody, a Fab-fusion protein or a Fc-fusion proteins) as disclosed herein. The expression vector comprises a nucleic acid encoding the protein comprising the Fc domain and/or the Fab domain (e.g. an antibody, a Fab-fusion protein or a Fc-fusion proteins) disclosed herein. In embodiments, the expression vector comprises DNA or RNA. In embodiments, the expression vector is a mammalian expression vector.

Both prokaryotic and eukaryotic vectors can be used for expression of the protein comprising the Fc domain and/or the Fab domain (e.g. an antibody, a Fab-fusion protein or a Fc-fusion proteins). Prokaryotic vectors include constructs based on E. coli sequences (see, e.g., Makrides, Microbiol Rev 1996, 60:512-538). Non-limiting examples of regulatory regions that can be used for expression in E. coli include lac, trp, Ipp, phoA, recA, tac, T3, T7 and APL. Non-limiting examples of prokaryotic expression vectors may include the λgt vector series such as λgt11 (Huynh et al., in “DNA Cloning Techniques, Vol. I: A Practical Approach,” 1984, (D. Glover, ed.), pp. 49-78, IRL Press, Oxford), and the pET vector series (Studier et al., Methods Enzymol 1990, 185:60-89). Prokaryotic host-vector systems cannot perform much of the post-translational processing of mammalian cells, however. Thus, eukaryotic host-vector systems may be particularly useful. A variety of regulatory regions can be used for expression of the proteins comprising the Fc domain and/or the Fab domain (e.g. an antibody, a Fab-fusion protein or a Fc-fusion proteins) in mammalian host cells. For example, the SV40 early and late promoters, the cytomegalovirus (CMV) immediate early promoter, and the Rous sarcoma virus long terminal repeat (RSV-LTR) promoter can be used. Inducible promoters that may be useful in mammalian cells include, without limitation, promoters associated with the metallothionein II gene, mouse mammary tumor virus glucocorticoid responsive long terminal repeats (MMTV-LTR), the β-interferon gene, and the hsp70 gene (see, Williams et al., Cancer Res 1989, 49:2735-42; and Taylor et al., Mol Cell Biol 1990, 10:165-75). Heat shock promoters or stress promoters also may be advantageous for driving expression of the proteins comprising the Fc domain and/or the Fab domain (e.g. an antibody, a Fab-fusion protein or a Fc-fusion proteins) in recombinant host cells.

In embodiments, the present invention contemplates the use of inducible promoters capable of effecting high level of expression transiently in response to a cue. For example, when in the proximity of a tumor cell, a cell transformed with an expression vector for the protein comprising the Fc domain and/or the Fab domain (e.g. an antibody, a Fab-fusion protein or a Fc-fusion proteins) comprising such an expression control sequence is induced to transiently produce a high level of the agent by exposing the transformed cell to an appropriate cue. Illustrative inducible expression control regions include those comprising an inducible promoter that is stimulated with a cue such as a small molecule chemical compound. Particular examples can be found, for example, in U.S. Pat. Nos. 5,989,910, 5,935,934, 6,015,709, and 6,004,941, each of which is incorporated herein by reference in its entirety.

Expression control regions and locus control regions include full-length promoter sequences, such as native promoter and enhancer elements, as well as subsequences or polynucleotide variants which retain all or part of full-length or non-variant function. As used herein, the term “functional” and grammatical variants thereof, when used in reference to a nucleic acid sequence, subsequence or fragment, means that the sequence has one or more functions of native nucleic acid sequence (e.g., non-variant or unmodified sequence).

Expression systems functional in human cells are well known in the art, and include viral systems. Generally, a promoter functional in a human cell is any DNA sequence capable of binding mammalian RNA polymerase and initiating the downstream (3′) transcription of a coding sequence into mRNA. A promoter will have a transcription initiating region, which is usually placed proximal to the 5′ end of the coding sequence, and typically a TATA box located 25-30 base pairs upstream of the transcription initiation site. The TATA box is thought to direct RNA polymerase II to begin RNA synthesis at the correct site. A promoter will also typically contain an upstream promoter element (enhancer element), typically located within 100 to 200 base pairs upstream of the TATA box. An upstream promoter element determines the rate at which transcription is initiated and can act in either orientation. Of particular use as promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40. Introns may also be included in expression constructs.

There are varieties of techniques available for introducing nucleic acids into viable cells. Techniques suitable for the transfer of nucleic acid into mammalian cells in vitro include the use of liposomes, electroporation, microinjection, cell fusion, polymer-based systems, DEAE-dextran, viral transduction, the calcium phosphate precipitation method, etc. For in vivo gene transfer, a number of techniques and reagents may also be used, including liposomes; natural polymer-based delivery vehicles, such as chitosan and gelatin; viral vectors are also suitable for in vivo transduction. In some situations, it is desirable to provide a targeting agent, such as an antibody or ligand specific for a tumor cell surface membrane protein. Where liposomes are employed, proteins which bind to a cell surface membrane protein associated with endocytosis may be used for targeting and/or to facilitate uptake, e.g., capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990).

Where appropriate, gene delivery agents such as, e.g., integration sequences can also be employed. Numerous integration sequences are known in the art (see, e.g., Nunes-Duby et al., Nucleic Acids Res. 26:391-406, 1998; Sadwoski, J. Bacteriol., 165:341-357, 1986; Bestor, Cell, 122(3):322-325, 2005; Plasterk et al., TIG 15:326-332, 1999; Kootstra et al., Ann. Rev. Pharm. Toxicol., 43:413-439, 2003). These include recombinases and transposases. Examples include Cre (Sternberg and Hamilton, J. Mol.

Biol., 150:467-486, 1981), lambda (Nash, Nature, 247, 543-545, 1974), Flp (Broach, et al., Cell, 29:227-234, 1982), R (Matsuzaki, et al., J. Bacteriology, 172:610-618, 1990), cpC31 (see, e.g., Groth et al., J. Mol. Biol. 335:667-678, 2004), sleeping beauty, transposases of the mariner family (Plasterk et al., supra), and components for integrating viruses such as AAV, retroviruses, and antiviruses having components that provide for virus integration such as the LTR sequences of retroviruses or lentivirus and the ITR sequences of AAV (Kootstra et al., Ann. Rev. Pharm. Toxicol., 43:413-439, 2003). In addition, direct and targeted genetic integration strategies may be used to insert nucleic acid sequences encoding the proteins comprising the Fc domain and/or the Fab domain (e.g. an antibody, a Fab-fusion protein or a Fc-fusion proteins) including CRISPR/CAS9, zinc finger, TALEN, and meganuclease gene-editing technologies.

In embodiments, the expression vectors for the expression of the proteins comprising the Fc domain and/or the Fab domain (e.g. an antibody, a Fab-fusion protein or a Fc-fusion proteins) are viral vectors. Many viral vectors useful for gene therapy are known (see, e.g., Lundstrom, Trends Biotechnol., 21: 1 17, 122, 2003. Illustrative viral vectors include those selected from Antiviruses (LV), retroviruses (RV), adenoviruses (AV), adeno-associated viruses (AAV), and a viruses, though other viral vectors may also be used. For in vivo uses, viral vectors that do not integrate into the host genome are suitable for use, such as a viruses and adenoviruses. Illustrative types of a viruses include Sindbis virus, Venezuelan equine encephalitis (VEE) virus, and Semliki Forest virus (SFV). For in vitro uses, viral vectors that integrate into the host genome are suitable, such as retroviruses, AAV, and Antiviruses. In embodiments, the invention provides methods of transducing a human cell in vivo, comprising contacting a solid tumor in vivo with a viral vector of the invention.

Aspects of the present invention include a host cell comprising the expression vector which comprises the protein comprising the Fc domain and/or the Fab domain (e.g. an antibody, a Fab-fusion protein or a Fc-fusion proteins) disclosed herein.

Expression vectors can be introduced into host cells for producing the present proteins comprising the Fc domain and/or the Fab domain (e.g. an antibody, a Fab-fusion protein or a Fc-fusion proteins). Cells may be cultured in vitro or genetically engineered, for example. Useful mammalian host cells include, without limitation, cells derived from humans, monkeys, and rodents (see, for example, Kriegler in “Gene Transfer and Expression: A Laboratory Manual,” 1990, New York, Freeman & Co.). These include monkey kidney cell lines transformed by SV40 (e.g., COS-7, ATCC CRL 1651); human embryonic kidney lines (e.g., 293, 293-EBNA, or 293 cells subcloned for growth in suspension culture, Graham et al., J Gen Virol 1977, 36:59); baby hamster kidney cells (e.g., BHK, ATCC CCL 10); Chinese hamster ovary-cells-DHFR (e.g., CHO, Urlaub and Chasin, Proc Natl Acad Sci USA 1980, 77:4216); DG44 CHO cells, CHO-K1 cells, mouse sertoli cells (Mather, Biol Reprod 1980, 23:243-251); mouse fibroblast cells (e.g., NIH-3T3), monkey kidney cells (e.g., CV1 ATCC CCL 70); African green monkey kidney cells. (e.g., VERO-76, ATCC CRL-1587); human cervical carcinoma cells (e.g., HELA, ATCC CCL 2); canine kidney cells (e.g., MDCK, ATCC CCL 34); buffalo rat liver cells (e.g., BRL 3A, ATCC CRL 1442); human lung cells (e.g., W138, ATCC CCL 75); human liver cells (e.g., Hep G2, HB 8065); and mouse mammary tumor cells (e.g., MMT 060562, ATCC CCL51). Illustrative cancer cell types for expressing the proteins comprising the Fc domain and/or the Fab domain (e.g. an antibody, a Fab-fusion protein or a Fc-fusion proteins) disclosed herein include mouse fibroblast cell line, NIH3T3, mouse Lewis lung carcinoma cell line, LLC, mouse mastocytoma cell line, P815, mouse lymphoma cell line, EL4 and its ovalbumin transfectant, E.G7, mouse melanoma cell line, B16F10, mouse fibrosarcoma cell line, MC57, and human small cell lung carcinoma cell lines, SCLC #2 and SCLC #7.

Host cells can be obtained from normal or affected subjects, including healthy humans, cancer patients, and patients with an infectious disease, private laboratory deposits, public culture collections such as the American Type Culture Collection (ATCC), or from commercial suppliers.

Cells that can be used for production of the present proteins comprising the Fc domain and/or the Fab domain (e.g. an antibody, a Fab-fusion protein or a Fc-fusion proteins) in vitro, ex vivo, and/or in vivo include, without limitation, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells (e.g., as obtained from bone marrow), umbilical cord blood, peripheral blood, and fetal liver. The choice of cell type depends on the type of tumor or infectious disease being treated or prevented, and can be determined by one of skill in the art.

According to the present invention, proteins comprising the Fc domain and/or the Fab domain (e.g. an antibody, a Fab-fusion protein or a Fc-fusion proteins) may be purified using specific solid substrates/solid supports, e.g., beads and chromatography resins, or using chromatography methods that do not depend upon Protein A capture. In embodiments, the proteins comprising the Fc domain and/or the Fab domain (e.g. an antibody, a Fab-fusion protein or a Fc-fusion proteins) may be purified in an oligomeric state, or in multiple oligomeric states, and enriched for a specific oligomeric state using specific methods. Without being bound by theory, these methods could include treatment with specific buffers including specified salt concentrations, pH and additive compositions. In other examples, such methods could include treatments that favor one oligomeric state over another. The proteins comprising the Fc domain and/or the Fab domain (e.g. an antibody, a Fab-fusion protein or a Fc-fusion proteins) obtained herein may be additionally ‘polished’ using methods that are specified in the art. In embodiments, the proteins comprising the Fc domain and/or the Fab domain (e.g. an antibody, a Fab-fusion protein or a Fc-fusion proteins) are highly stable and able to tolerate a wide range of pH exposure (between pH 3-12), are able to tolerate a large number of freeze/thaw stresses (greater than 3 freeze/thaw cycles) and are able to tolerate extended incubation at high temperatures (longer than 2 weeks at 40 degrees C.). In embodiments, the proteins comprising the Fc domain and/or the Fab domain (e.g. an antibody, a Fab-fusion protein or a Fc-fusion proteins) are shown to remain intact, without evidence of degradation, deamidation, etc. under such stress conditions.

Isolated/purified proteins comprising the Fc domain and/or the Fab domain (e.g. an antibody, a Fab-fusion protein or a Fc-fusion proteins) may be stored in liquid form for some period of time, frozen for extended periods of time or in some cases lyophilized.

Subjects and/or Animals

In embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In embodiments, the subject and/or animal is a non-mammal, such, for example, a zebrafish. In embodiments, the subject and/or animal may comprise fluorescently-tagged cells (with e.g. GFP). In embodiments, the subject and/or animal is a transgenic animal comprising a fluorescent cell.

In embodiments, the subject and/or animal is a human. In embodiments, the human is a pediatric human. In embodiments, the human is an adult human. In embodiments, the human is a geriatric human. In embodiments, the human may be referred to as a patient.

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In embodiments, the subject is a non-human animal, and therefore the invention pertains to veterinary use. In a specific embodiment, the non-human animal is a household pet. In another specific embodiment, the non-human animal is a livestock animal.

### Methods of Treatment of Prevention

In specific embodiments, it may be desirable to administer locally to the area in need of treatment the proteins comprising the Fc domain and/or the Fab domain (e.g. an antibody, a Fab-fusion protein or a Fc-fusion proteins) purified according to the methods disclosed herein. In embodiments, for instance in the treatment of cancer, the proteins comprising the Fc domain and/or the Fab domain (e.g. an antibody, a Fab-fusion protein or a Fc-fusion proteins) purified according to the methods disclosed herein (and/or additional agents) are administered in the tumor microenvironment (e.g. cells, molecules, extracellular matrix and/or blood vessels that surround and/or feed a tumor cell, inclusive of, for example, tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T cells; macrophages; neutrophils; and other immune cells located proximal to a tumor) or lymph node and/or targeted to the tumor microenvironment or lymph node. In embodiments, for instance in the treatment of cancer, the proteins comprising the Fc domain and/or the Fab domain (e.g. an antibody, a Fab-fusion protein or a Fc-fusion proteins) purified according to the methods disclosed herein (and/or additional agents) are administered intratumorally.

In embodiments, the present proteins comprising the Fc domain and/or the Fab domain (e.g. an antibody, a Fab-fusion protein or a Fc-fusion proteins) purified according to the methods disclosed herein allows for a dual effect that provides less side effects than are seen in conventional immunotherapy (e.g. treatments with one or more of OPDIVO, KEYTRUDA, YERVOY, and TECENTRIQ). For example, the present proteins comprising the Fc domain and/or the Fab domain (e.g. an antibody, a Fab-fusion proteins or a Fc-fusion proteins) purified according to the methods disclosed herein reduce or prevent commonly observed immune-related adverse events that affect various tissues and organs including the skin, the gastrointestinal tract, the kidneys, peripheral and central nervous system, liver, lymph nodes, eyes, pancreas, and the endocrine system; such as hypophysitis, colitis, hepatitis, pneumonitis, rash, and rheumatic disease. Further, the present local administration, e.g. intratumorally, obviate adverse event seen with standard systemic administration, e.g. IV infusions, as are used with conventional immunotherapy (e.g. treatments with one or more of OPDIVO, KEYTRUDA, YERVOY, and TECENTRIQ).

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art.

In embodiments, delivery can be in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989).

Any proteins comprising the Fc domain and/or the Fab domain (e.g. an antibody, a Fab-fusion protein or a Fc-fusion proteins) purified according to the methods disclosed herein (and/or additional agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In embodiments, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

In embodiments, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Yet another aspect of the present invention provides a method for treating a cancer or an autoimmune disease in a subject in need thereof, the method comprising administering to the subject a therapeutically-effective amount of the isolated and/or purified proteins comprising the Fc domain and/or the Fab domain (e.g. an antibody, a Fab-fusion protein or a Fc-fusion proteins) purified according to the methods disclosed herein of any of the embodiments disclosed herein.

Yet another aspect of the present invention provides a method for treating a cancer or an autoimmune disease in a subject in need thereof, the method comprising administering to the subject a therapeutically-effective amount a proteins comprising the Fc domain and/or the Fab domain (e.g. an antibody, a Fab-fusion protein or a Fc-fusion proteins) purified according to the methods disclosed herein of any of the embodiments disclosed herein.

Yet another aspect of the present invention provides a method for treating a cancer or an autoimmune disease in a subject in need thereof, the method comprising administering to the subject a therapeutically-effective amount of the composition of any of the embodiments disclosed herein.

Yet another aspect of the present invention provides a method for treating a cancer or an autoimmune disease in a subject in need thereof, the method comprising administering to the subject a therapeutically-effective amount of an isolated and/or purified proteins comprising the Fc domain and/or the Fab domain (e.g. an antibody, a Fab-fusion protein or a Fc-fusion proteins) purified according to the methods disclosed herein prepared using the method of any of the embodiments disclosed herein.

Yet another aspect of the present invention provides a method for treating a cancer or an autoimmune disease in a subject in need thereof, the method comprising administering to the subject a therapeutically-effective amount of the composition of any of the embodiments disclosed herein.

Yet another aspect of the present invention provides a method for treating a cancer or an autoimmune disease in a subject in need thereof, the method comprising administering to the subject a therapeutically-effective amount of an isolated and/or purified proteins comprising the Fc domain and/or the Fab domain (e.g. an antibody, a Fab-fusion protein or a Fc-fusion proteins) purified according to the methods disclosed herein prepared using the method of any of the embodiments disclosed herein.

Yet another aspect of the present invention provides a method for preventing or treating an infectious disease in a subject in need thereof, the method comprising administering to the subject a therapeutically-effective amount of the composition of any of the embodiments disclosed herein.

Yet another aspect of the present invention provides a method for preventing or treating an infectious disease in a subject in need thereof, the method comprising administering to the subject a therapeutically-effective amount of an isolated and/or purified proteins comprising the Fc domain and/or the Fab domain (e.g. an antibody, a Fab-fusion protein or a Fc-fusion proteins) purified according to the methods disclosed herein prepared using the method of any of the embodiments disclosed herein.

Yet another aspect of the present invention provides a method for preventing or treating an infectious disease in a subject in need thereof, the method comprising administering to the subject a therapeutically-effective amount of the composition of any of the embodiments disclosed herein.

Yet another aspect of the present invention provides a method for preventing or treating an infectious disease in a subject in need thereof, the method comprising administering to the subject a therapeutically-effective amount of an isolated and/or purified proteins comprising the Fc domain and/or the Fab domain (e.g. an antibody, a Fab-fusion protein or a Fc-fusion proteins) purified according to the methods disclosed herein prepared using the method of any of the embodiments disclosed herein. Any aspect or embodiment disclosed herein can be combined with any other aspect or embodiment as disclosed herein.

In one aspect, the present disclosure relates to a composition comprising an isolated and/or purified chimeric protein prepared using the method of any of the embodiments disclosed herein.

In one aspect, the present disclosure relates to a pharmaceutical composition comprising the isolated and/or purified chimeric protein of any of the embodiments disclosed herein, and a pharmaceutically acceptable excipient.

In one aspect, the present disclosure relates to a pharmaceutical composition comprising an isolated and/or purified chimeric protein prepared using the method of any of the embodiments disclosed herein, and a pharmaceutically acceptable excipient.

In one aspect, the present disclosure relates to a method for treating a cancer or an autoimmune disease in a subject in need thereof, the method comprising administering to the subject a therapeutically-effective amount of the isolated and/or purified chimeric protein of any of the embodiments disclosed herein.

In one aspect, the present disclosure relates to a method for treating a cancer or an autoimmune disease in a subject in need thereof, the method comprising administering to the subject a therapeutically-effective amount a chimeric protein of any of the embodiments disclosed herein.

In one aspect, the present disclosure relates to a method for treating a cancer or an autoimmune disease in a subject in need thereof, the method comprising administering to the subject a therapeutically-effective amount of the composition of any of the embodiments disclosed herein.

In one aspect, the present disclosure relates to a method for treating a cancer or an autoimmune disease in a subject in need thereof, the method comprising administering to the subject a therapeutically-effective amount of an isolated and/or purified chimeric protein prepared using the method of any one of any of the embodiments disclosed herein.

In one aspect, the present disclosure relates to a method for treating a cancer or an autoimmune disease in a subject in need thereof, the method comprising administering to the subject a therapeutically-effective amount of a chimeric protein having the structure: selected from: X-Linker and Linker-Y, wherein the X and the Y is selected from a mammalian secreted protein, mammalian intracellular protein, a mammalian membrane protein, an antigen, or a fragment thereof, and the linker comprises an Fc domain, wherein the chimeric protein is isolated and/or purified chimeric protein prepared using the method of any of the embodiments disclosed herein.

In one aspect, the present disclosure relates to a method for treating a cancer or an autoimmune disease in a subject in need thereof, the method comprising administering to the subject a therapeutically-effective amount of a chimeric protein having the structure: X-Linker-Y, wherein X and/or Y is independently selected from a mammalian secreted protein, mammalian intracellular protein, a mammalian membrane protein, an antigen, or a fragment thereof, and the linker comprises an Fc domain, wherein the chimeric protein is isolated and/or purified chimeric protein prepared using the method of any of the embodiments disclosed herein.

Any aspect or embodiment disclosed herein can be combined with any other aspect or embodiment as disclosed herein.

### Select Embodiments

Embodiment 1. A method for isolating and/or purifying a chimeric protein from a solution, wherein the chimeric protein has the structure selected from:
X-Linker
   and
Linker-Y
wherein the X or the Y is selected from a mammalian secreted protein, mammalian intracellular protein, a mammalian membrane protein, an antigen, or a fragment thereof, and
the linker comprises an Fc domain,
the method comprising:
(a) providing a solution comprising the chimeric protein, the solution further comprising one or more host cell proteins (HCPs),
(b) contacting the solution comprising the chimeric protein with a polypeptide capable of binding clusterin to produce a mixture, and
(c) removing the polypeptide capable of binding clusterin from the mixture, and thereby removing free clusterin, clusterin bound to the polypeptide, and/or any additional HCPs contemporaneously bound to clusterin and/or the polypeptide from the mixture, and thereby isolating and/or purifying the chimeric protein.

Embodiment 2. A method for isolating and/or purifying a chimeric protein from a solution, wherein the chimeric protein has the structure:
X-Linker-Y
wherein the X and the Y are independently absent or selected from a mammalian secreted protein, mammalian intracellular protein, a mammalian membrane protein, an antigen, or a fragment thereof, and
the linker comprises an Fc domain,
the method comprising:
(a) providing a solution comprising the chimeric protein, the solution further comprising one or more host cell proteins (HCPs),
(b) contacting the solution comprising the chimeric protein with a polypeptide capable of binding clusterin to produce a mixture, and
(c) removing the polypeptide capable of binding clusterin from the mixture, and thereby removing free clusterin, clusterin bound to the polypeptide, and/or any additional HCPs contemporaneously bound to clusterin and/or the polypeptide from the mixture, and thereby isolating and/or purifying the chimeric protein.

Embodiment 3. The method of embodiment 1 or embodiment 2, wherein the polypeptide capable of binding clusterin is conjugated to a moiety selected from a magnetic bead, a tag, and combination thereof.

Embodiment 4. The method of any one of embodiments 1 to 3, wherein the polypeptide capable of binding clusterin is conjugated to a magnetic bead and the step (c) comprises:
(i) contacting the solution with a magnetic field, and
(ii) recovering a second solution comprising the chimeric protein, the second solution being substantially free of the polypeptide capable of binding clusterin and/or the magnetic bead.

Embodiment 5. The method of any one of embodiments 1 to 3, wherein the polypeptide capable of binding clusterin is conjugated to a tag and the method further comprises
(i) contacting the solution with a tag-binding agent, optionally wherein the tag-binding agent is immobilized on a solid support, and
(ii) recovering a second solution comprising the chimeric protein, the second solution being substantially free of the polypeptide capable of binding clusterin and/or the tag.

Embodiment 6. A method for isolating and/or purifying a chimeric protein from a solution, wherein the chimeric protein has the structure selected from:
X-Linker
   and
Linker-Y
wherein the X and the Y is selected from a mammalian secreted protein, mammalian intracellular protein, a mammalian membrane protein, or a fragment thereof, and
the linker comprises an Fc domain,
the method comprising:
(a) providing a solution comprising the chimeric protein, the solution further comprising one or more host cell proteins (HCP),
(b) contacting the solution comprising the chimeric protein with a polypeptide immobilized onto the surface of a solid support, wherein the immobilized polypeptide is capable of binding clusterin, and
(c) removing free clusterin, clusterin bound to the polypeptide, and any additional HCPs contemporaneously bound to clusterin and/or the polypeptide from the solution, thereby isolating and/or purifying the chimeric protein.

Embodiment 7. A method for isolating and/or purifying a chimeric protein from a solution, wherein the chimeric protein has the structure:
X-Linker-Y
wherein X and Y are independently absent or selected from a mammalian secreted protein, mammalian intracellular protein, a mammalian membrane protein, or a fragment thereof, and
the linker comprises an Fc domain, the method comprising:
(a) providing a solution comprising the chimeric protein, the solution further comprising one or more host cell proteins (HCP),
(b) contacting the solution comprising the chimeric protein with a polypeptide immobilized onto the surface of a solid support, wherein the immobilized polypeptide is capable of binding clusterin, and
(c) removing free clusterin, clusterin bound to the polypeptide, and any additional HCPs contemporaneously bound to clusterin and/or the polypeptide from the solution, thereby isolating and/or purifying the chimeric protein.

Embodiment 8. The method of embodiment 6 or embodiment 7, wherein the polypeptide is immobilized on a chromatography resin.

Embodiment 9. The method of embodiment 8, wherein the chromatography resin is located in a chromatography column.

Embodiment 10. The method of any one of embodiments 6 to 9, wherein the flow through sample of step (c) is recovered.

Embodiment 11. The method of embodiment 9, optionally wherein the chromatography column is washed and a second flow through sample is recovered.

Embodiment 12. The method of embodiment 11, optionally wherein the flow through sample and the second flow through sample are mixed to produce a combined flow through sample.

Embodiment 13. The method of any one of embodiments 6 to 12, wherein the step (c) removes at least one HCP, optionally wherein the at least one HCP comprises clusterin and at least one more HCP.

Embodiment 14. The method of embodiment 13, wherein the at least one HCP comprises clusterin.

Embodiment 15. The method of embodiment 14, wherein the at least one HCP further comprises a contaminant that binds to clusterin.

Embodiment 16. The method of embodiment 12, wherein the flow through sample and the second flow through sample and/or the combined flow through sample comprises fewer contaminants than the solution comprising chimeric protein.

Embodiment 17. The method of embodiment 16, wherein the contaminants comprise components of a mammalian cell harboring a nucleic acid that expresses the chimeric protein.

Embodiment 18. The method of any one of embodiments 5 to 17, wherein the solid support is a bead or a chromatography resin.

Embodiment 19. The method of embodiment 18, wherein the bead is an agarose bead.

Embodiment 20. The method of embodiment 19, wherein the agarose bead is an aldehyde-activated agarose bead.

Embodiment 21. The method of any one of embodiments 18 to 20, wherein the immobilized polypeptide is coupled to the bead via a free NH₂.

Embodiment 22. The method of embodiment 18, wherein the chromatography resin comprises crosslinked poly[styrene divinylbenzene].

Embodiment 23. The method of any one of embodiments 1 to 22, wherein the solution comprising the chimeric protein is selected from an eluate from a chromatography step, a culture supernatant, and a cell-free extract.

Embodiment 24. The method of embodiment 23, wherein the chromatography step is selected from an affinity chromatography, an ion exchange chromatography, a hydrophobic interaction chromatography, a reverse phase chromatography and a size exclusion chromatography.

Embodiment 25. The method of embodiment 23 or embodiment 24, wherein the chromatography step is an affinity chromatography.

Embodiment 26. The method of embodiment 23 or embodiment 24, wherein the chromatography step is an ion exchange chromatography.

Embodiment 27. The method of embodiment 23 or embodiment 24, wherein the chromatography step is a hydrophobic interaction chromatography.

Embodiment 28. The method of any one of embodiments 23 to 27, wherein the culture supernatant is derived from culture of mammalian cells expressing chimeric protein.

Embodiment 29. The method of any one of embodiments 23 to 27, wherein the culture supernatant is derived from culturing a cell line expressing chimeric protein.

Embodiment 30. The method of embodiment 29, wherein the cell line is selected from NS0 murine myeloma cells, PER.C6 human cells, and Chinese hamster ovary (CHO) cells, baby hamster kidney (BHK21) cells, murine myeloma Sp2/0 cells, human embryonic kidney 293 (HEK293) cells, HT-1080 cells, Hela cells, CAP cells, HKB-11 cells, HuH-7 cells, Sf9 cells, and a derivative thereof.

Embodiment 31. The method of embodiment 29 or embodiment 30, wherein the cell line is selected from CHO DUXB11, CHO DG44, CHOK1, ExpiCHO and Expi293.

Embodiment 32. The method of embodiment 23, wherein the cell-free extract is derived from culture of mammalian cells expressing chimeric protein.

Embodiment 33. The method of embodiment 32, wherein the cell-free extract is derived from culturing a cell line expressing chimeric protein.

Embodiment 34. The method of embodiment 33, wherein the cell line is selected from NS0 murine myeloma cells, PER.C6 human cells, and Chinese hamster ovary (CHO) cells, baby hamster kidney (BHK21) cells, murine myeloma Sp2/0 cells, human embryonic kidney 293 (HEK293) cells, HT-1080 cells, Hela cells, CAP cells, HKB-11 cells, HuH-7 cells, Sf9 cells, and a derivative thereof.

Embodiment 35. The method of embodiment 33 or embodiment 34, wherein the cell line is selected from CHO DUXB11, CHO DG44, CHOK1, ExpiCHO and Expi293.

Embodiment 36. The method of any one of embodiments 1 to 35, wherein the Fc domain is a mammalian Fc domain.

Embodiment 37. The method of embodiment 36, wherein the chimeric protein comprises a Fc domain selected from a human Fc domain, a mouse Fc domain, a rat Fc domain, a sheep Fc domain, a goat Fc domain, a donkey Fc domain, a feline Fc domain a hamster Fc domain, a guinea pig Fc domain, a horse Fc domain, a rabbit Fc domain, a pig Fc domain, a dog Fc domain, and a cow Fc domain.

Embodiment 38. The method of embodiment 36, wherein the chimeric protein comprises a human Fc domain.

Embodiment 39. The method of any one of embodiments 1 to 38, wherein the chimeric protein comprises a Fc domain selected from an IgG Fc domain, an IgA Fc domain, an IgM Fc domain, an IgE Fc domain and an IgD Fc domain.

Embodiment 40. The method of embodiment 39, wherein the IgG Fc domain is selected from an IgG1 Fc domain, an IgG2 Fc domain, an IgG3 Fc domain, and an IgG4 Fc domain.

Embodiment 41. The method of embodiment 39, wherein the IgA is selected from an IgA1 and an IgA2.

Embodiment 42. The method of any one of embodiments 1 to 41, wherein the X and/or Y is independently an antigen, a mammalian intracellular protein, a mammalian secreted protein, a mammalian membrane protein, or a fragment thereof.

Embodiment 43. The method of embodiment 42, wherein the X and/or Y is an antigen, wherein the antigen is derived from a pathogen.

Embodiment 44. The method of embodiment 43, wherein the pathogen is selected from a virus, a bacterium, a protozoan, a parasite, and a fungus.

Embodiment 45. The method of embodiment 42, wherein the antigen is a cancer antigen.

Embodiment 46. The method of embodiment 45, wherein the cancer antigen is a neoantigen.

Embodiment 47. The method of any one of embodiments 1 to 41, wherein the X and/or Y is a mammalian intracellular protein, or a fragment thereof.

Embodiment 48. The method of any one of embodiments 1 to 41, wherein the X and/or Y is a mammalian secreted protein, or a fragment thereof.

Embodiment 49. The method of embodiment 48, wherein the secreted protein is a cytokine.

Embodiment 50. The method of embodiment 49, wherein the cytokine is selected from IFN-α, IFN-β, IFN-ε, IFN-κ, IFN-ω IFN-γ, IL-1α, IL-1p, IL-1, IL-2, IL-3, IL-4, IL-5, IL-6, IL-7, IL-9, IL-10, IL-11, IL-12, IL-13, IL-15, IL-16, IL-17, IL-18, IL-19, IL-20, IL-35, TNF, leukemia inhibitory factor (LIF), oncostatin M (OSM), granulocyte colony-stimulating factor (G-CSF), TNF-α, TNF-β, lymphotoxin (LT)-β, LIGHT, Fas ligand (FasL)/CD178, TGF-β1, TGF-β2, TGF-β3, XCL1, XCL2, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CXCL1, CXCL2, CXCL3, CXCL4, CXCL5, CXCL6, CXCL7, CXCL8, CXCL9, CXCL10, CXCL11, CXCL12, CXCL13, CXCL14, CX3CL1, Epo, Tpo, SCF, and FLT-3L.

Embodiment 51. The method of any one of embodiments 1 to 41, wherein the X and/or Y is a mammalian membrane protein, or a fragment thereof.

Embodiment 52. The method of embodiment 51, wherein the mammalian membrane protein is selected from SLAMF4, IL-2Rα, 4-1BB/TNFRSF9, IL-2R β, ALCAM, B7-1, IL-4 R, B7-H3, BLAME/SLAMFS, CEACAM1, IL-6 R, IL-7 Rα, IL-10Rα, IL-10R β, IL-12R β 1, IL-12Rβ2, CD2, IL-13 Rα1, IL-13, CD3, CD4, ILT2/CDS5j, ILT3/CDS5k, ILT4/CDS5d, ILT5/CDS5a, Integrin α 4/CD49d, CDS, Integrin α E/CD103, CD6, Integrin α M/CD11b, CDS, Integrin α X/CD11c, Integrin β 2/CDIS, KIR/CD15S, CD27/TNFRSF7, KIR2DL1, CD2S, KIR2DL3, CD30/TNFRSFS, KIR2DL4/CD15Sd, CD31/PECAM-1, KIR2DS4, CD40 Ligand/TNFSF5, LAG-3, CD43, LAIR1, CD45, LAIR2, CDS3, Leukotriene B4-R1, CDS4/SLAMF5, NCAM-L1, CD94, NKG2A, CD97, NKG2C, CD229/SLAMF3, NKG2D, CD2F-10/SLAMF9, NT-4, CD69, NTB-A/SLAMF6, Common γ Chain/lL-2 R γ, Osteopontin, CRACC/SLAMF7, PD-1, CRTAM, PSGL-1, CTLA-4, RANK/TNFRSF11A, CX3CR1, CX3CL1, L-Selectin, SIRP p 1, SLAM, TCCR/WSX-1, DNAM-1, Thymopoietin, EMMPRIN/CD147, TIM-1, EphB6, TIM-2, Fas/TNFRSF6, TIM-3, Fas Ligand/TNFSF6, TIM-4, Fcγ RIII/CD16, TIM-6, TNFR1/TNFRSF1A, Granulysin, TNF RIII/TNFRSF1B, TRAIL RI/TNFRSFIOA, ICAM-1/CD54, TRAIL R2/TNFRSF10B, ICAM-2/CD102, TRAILR3/TNFRSF10C, IFN-γR1, TRAILR4/TNFRSF10D, IFN-γ R2, TSLP, IL-1 R1 and TSLP R, or a fragment thereof.

Embodiment 53. The method of embodiment 51, wherein X and/or Y is a mammalian membrane protein is a Type I membrane protein, or a fragment thereof.

Embodiment 54. The method of embodiment 53, wherein the Type I membrane protein is selected from TIM-3, BTLA, PD-1, CTLA-4, LAG-3, CD244, CSF1R, CD160, TIGIT, SIRPα/CD172a, 2B4, VISTA, VSIG8, LAG3, CD200, B7-H3, BTNL3, BTNL8, BTN2A1, BTN3A1, BTN3A2, NKG2A, NKG2B, NKG2C, NKG2D, NKG2E, NKG2F, and NKG2H, and TMIGD2, or a fragment thereof.

Embodiment 55. The method of embodiment 54, wherein the fragment of the Type I membrane protein is the extracellular domain or the ligand binding portion thereof.

Embodiment 56. The method of embodiment 51, wherein Y is a mammalian membrane protein is a Type II membrane protein, or a fragment thereof.

Embodiment 57. The method of embodiment 56, wherein the Type II membrane protein is selected from OX-40 ligand (OX-40L), LIGHT (CD258), GITR ligand (GITRL), 4-1BB Ligand (4-1BBL), CD70, CD30 ligand (CD30L), CD40 ligand (CD40L), a C-type lectin domain (CLEC) family member, CD137 ligand, RANKL, TRAIL, FasL, TL1A, CD80, CD86, CD58, PD-1, SLAMF6, SIRPα and TGFBR2, or a fragment thereof.

Embodiment 58. The method of embodiment 56 or embodiment 57, wherein the fragment of the Type II membrane protein is the extracellular domain thereof.

Embodiment 59. The method of 56 or embodiment 57, wherein the fragment of the Type II membrane protein is the ligand binding portion thereof.

Embodiment 60. The method of one of embodiments 42 to 59, wherein the fusion protein is capable of modulating an immune response.

Embodiment 61. The method of any one of embodiments 42 to 46 or embodiment 60, wherein the fusion protein is a vaccine.

Embodiment 62. The method of any one of embodiments 1 to 61, wherein the polypeptide capable of binding clusterin is selected from an antibody, a single-domain antibody, a heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂, or a binding fragment thereof.

Embodiment 63. The method of embodiment 62, wherein the polypeptide capable of binding clusterin is selected from an antibody, a single-domain antibody, a heavy-chain-only antibody (VHH), a single-chain antibody (scFv), or a binding fragment thereof.

Embodiment 64. The method of embodiment 62 or embodiment 63, wherein the polypeptide capable of binding clusterin is a recombinant heavy-chain-only antibody (VHH), or a binding fragment thereof.

Embodiment 65. The method of embodiment 64, wherein the VHH, or a binding fragment thereof, is a recombinant protein.

Embodiment 66. The method of any one of embodiments 1 to 65, wherein the method further comprises at least one more purification step.

Embodiment 67. The method of embodiment 66, wherein the at least one more purification step is liquid chromatography.

Embodiment 68. The method of embodiment 67, wherein the chromatography is an affinity chromatography.

Embodiment 69. The method of embodiment 68, wherein the affinity chromatography comprises contacting the solution comprising the chimeric protein with a moiety having affinity for the X domain, the Y domain and/or the Fc domain.

Embodiment 70. The method of embodiment 69, wherein the moiety having affinity for the X domain is an antibody, or a binding fragment thereof, or an antibody-like molecule or a binding fragment thereof.

Embodiment 71. The method of embodiment 70, wherein the antibody-like molecule is selected from an antibody, a single-domain antibody, a heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂, or a binding fragment thereof.

Embodiment 72. The method of 69, wherein the moiety having affinity for the Y domain is an antibody, or a binding fragment thereof, or an antibody-like molecule or a binding fragment thereof.

Embodiment 73. The method of embodiment 72, wherein the antibody-like molecule is selected from an antibody, a single-domain antibody, a heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂, or a binding fragment thereof.

Embodiment 74. The method of embodiment 69, wherein the moiety having affinity for the Fc domain is an antibody, or a binding fragment thereof, or an antibody-like molecule or a binding fragment thereof.

Embodiment 75. The method of embodiment 74, wherein the antibody-like molecule is selected from an antibody, a single-domain antibody, a heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂, or a Fc domain binding fragment thereof.

Embodiment 76. The method of embodiment 69, wherein the moiety having affinity for the Fc domain is selected from a protein A, a protein G, protein L, protein M, or a derivative thereof.

Embodiment 77. The method of embodiment 68, wherein the affinity chromatography is selected from FcXL chromatography, protein A chromatography, a protein G chromatography, protein L chromatography, and protein M chromatography.

Embodiment 78. The method of any one of embodiments 68 to 77, wherein the affinity chromatography comprises:
contacting a solution comprising the chimeric protein with the moiety having affinity for the X domain, the Y domain and/or the Fc domain that is conjugated to a solid support, and
thereby directly or indirectly attaching the chimeric protein and/or at least one HCP to the solid support.

Embodiment 79. The method of embodiment 78, wherein the solution comprising the chimeric protein is selected from an eluate from a chromatography step, a flow through sample of a chromatography step, a culture supernatant, and a cell-free extract.

Embodiment 80. The method of embodiment 79, wherein the eluate from the chromatography step is selected from an eluate from an ion exchange chromatography, a hydrophobic interaction chromatography, a reverse phase chromatography and a size exclusion chromatography.

Embodiment 81. The method of embodiment 79, wherein the flow through sample of the chromatography step is a flow through from a clusterin scavenging chromatography.

Embodiment 82. The method of embodiment 81, wherein the clusterin is scavenged using a polypeptide immobilized onto the surface of a solid support, wherein the immobilized polypeptide is capable of binding clusterin.

Embodiment 83. The method of embodiment 82, wherein the polypeptide capable of binding clusterin is selected from an antibody, a single-domain antibody, a heavy-chain-only antibody (VHH), a single-chain antibody (scFv), or a binding fragment thereof.

Embodiment 84. The method of embodiment 78, wherein the solid support is a bead or a chromatography resin.

Embodiment 85. The method of embodiment 84, wherein the bead is an agarose bead.

Embodiment 86. The method of embodiment 85, wherein the agarose bead is an aldehyde-activated agarose bead.

Embodiment 87. The method of any one of embodiments embodiment 84 to 86, wherein the immobilized polypeptide is coupled to the bead via a free NH₂.

Embodiment 88. The method of embodiment 84, wherein the chromatography resin comprises crosslinked poly[styrene divinylbenzene].

Embodiment 89. The method of any one of embodiments 69 to 88, wherein the moiety having affinity for the X domain, the Y domain and/or the Fc domain that is conjugated to the solid support is located in a second chromatography column.

Embodiment 90. The method of embodiment 89, wherein the second chromatography column is washed with a buffer.

Embodiment 91. The method of embodiment 90, wherein the buffer comprises an ingredient selected from a salt, a detergent, an alcohol, a buffering agent and a combination thereof.

Embodiment 92. The method of embodiment 91, wherein the buffer is ice cold.

Embodiment 93. The method of any one of embodiments 90 to 92, wherein the wash with the buffer removes the chimeric protein and/or the at least one HCP that is indirectly attached to the solid support.

Embodiment 94. The method of any one of embodiments 68 to 93, further comprising contacting the chimeric protein that is directly attached to the solid support with a first elution solution that releases the X domain, the Fc domain, and/or the Y domain from the second chromatography column, thereby forming a first eluate which comprises the chimeric protein.

Embodiment 95. The method of any one of embodiments 68 to 94, wherein the affinity chromatography removes at least one HCP.

Embodiment 96. The method of embodiment 94, wherein the first eluate comprises fewer contaminants than the solution comprising the chimeric protein that was contacted with the moiety having affinity for the X domain, the Y domain and/or the Fc domain that is conjugated to the solid support.

Embodiment 97. The method of embodiment 96, wherein the contaminants comprise components of a mammalian cell that harbors a nucleic acid that expresses the chimeric protein.

Embodiment 98. The method of any one of embodiments 1 to 97, wherein the method further comprises at least one more purification step.

Embodiment 99. The method of embodiment 98, wherein the at least one more purification step is liquid chromatography.

Embodiment 100. The method of embodiment 99, wherein the chromatography is an ion exchange chromatography.

Embodiment 101. The method of embodiment 100, wherein the chromatography is an anion exchange chromatography or a cation exchange chromatography.

Embodiment 102. The method of embodiment 100, wherein the ion exchange chromatography comprises:
contacting a solution comprising the chimeric protein with an ion exchange matrix that is conjugated to a second solid support,
thereby directly or indirectly attaching the chimeric protein and/or at least one HCP to the second solid support.

Embodiment 103. The method of embodiment 102, wherein the solution comprising the chimeric protein is selected from the first eluate, an eluate from a chromatography step, a flow through sample of a chromatography step, a culture supernatant, and a cell-free extract.

Embodiment 104. The method of embodiment 103, wherein the eluate from the chromatography step is selected from an eluate from a hydrophobic interaction chromatography, a reverse phase chromatography and a size exclusion chromatography.

Embodiment 105. The method of embodiment 103, wherein the flow through sample of the chromatography step is a flow through from a clusterin scavenging chromatography.

Embodiment 106. The method of embodiment 105, wherein the clusterin is scavenged using a polypeptide immobilized onto the surface of a solid support, wherein the immobilized polypeptide is capable of binding clusterin.

Embodiment 107. The method of embodiment 106, wherein the polypeptide capable of binding clusterin is selected from an antibody, a single-domain antibody, a heavy-chain-only antibody (VHH), a single-chain antibody (scFv), or a binding fragment thereof.

Embodiment 108. The method of any one of embodiments 102 to 107, wherein the ion exchange matrix is selected from a weak carboxymethyl cation-exchanger, a strong sulfopropyl (SP) exchanger, and a weak diethylaminoethyl anion exchanger, and a strong quaternary aminoethyl (QAE) exchanger.

Embodiment 109. The method of any one of embodiments 102 to 108, wherein the solid support is a bead or a chromatography resin.

Embodiment 110. The method of embodiment 109, wherein the bead is an agarose bead.

Embodiment 111. The method of embodiment 110, wherein the agarose bead is an aldehyde-activated agarose bead.

Embodiment 112. The method of embodiment 111, wherein the immobilized polypeptide is coupled to the bead via a free NH₂.

Embodiment 113. The method of embodiment 109, wherein the chromatography resin comprises crosslinked poly[styrene divinylbenzene].

Embodiment 114. The method of any one of embodiments 102 to 113, wherein the ion exchange matrix that is conjugated to the second solid support is located in a third chromatography column.

Embodiment 115. The method of embodiment 114, wherein the third chromatography column is washed with a buffer.

Embodiment 116. The method of embodiment 115, wherein the buffer comprises an ingredient selected from a salt, a detergent, an alcohol, a buffering agent and a combination thereof.

Embodiment 117. The method of embodiment 116, wherein the buffer is ice cold.

Embodiment 118. The method of any one of embodiments 115 to 117, wherein the washing step the third chromatography column removes the chimeric protein and/or the at least one HCP that is indirectly attached to the second solid support.

Embodiment 119. The method of any one of embodiments 102 to 118, further comprising contacting the chimeric protein that is directly attached to the second solid support with a first elution solution that releases the chimeric protein from the second chromatography column, thereby forming a second eluate which comprises the chimeric protein.

Embodiment 120. The method of any one of embodiments 102 to 119, wherein the ion exchange chromatography removes at least one HCP.

Embodiment 121. The method of embodiment 119, wherein the second eluate comprises fewer contaminants than the solution comprising the chimeric protein that was contacted with the ion exchange matrix that is conjugated to a second solid support.

Embodiment 122. The method of embodiment 121, wherein the contaminants comprise components of a mammalian cell that harbors a nucleic acid that expresses the chimeric protein.

Embodiment 123. The method of any one of embodiments 1 to 122, wherein the method further comprises at least one more purification step.

Embodiment 124. The method of embodiment 123, wherein the at least one more purification step is liquid chromatography.

Embodiment 125. The method of embodiment 99, wherein the chromatography is a hydrophobic interaction chromatography.

Embodiment 126. The method of embodiment 125, wherein the hydrophobic interaction chromatography comprises:
contacting a solution comprising the chimeric protein with a hydrophobic interaction chromatography (HIC) media that is conjugated to a third solid support,
thereby directly or indirectly attaching the chimeric protein and/or at least one HCP to the third solid support.

Embodiment 127. The method of embodiment 126, wherein the solution comprising the chimeric protein is selected from the first eluate, the second eluate, an eluate from a chromatography step, a flow through sample of a chromatography step, a culture supernatant, and a cell-free extract.

Embodiment 128. The method of embodiment 127, wherein the eluate from the chromatography step is selected from an eluate from a reverse phase chromatography and a size exclusion chromatography.

Embodiment 129. The method of embodiment 127, wherein the flow through sample of the chromatography step is a flow through from a clusterin scavenging chromatography.

Embodiment 130. The method of embodiment 129, wherein the clusterin is scavenged using a polypeptide immobilized onto the surface of a solid support, wherein the immobilized polypeptide is capable of binding clusterin.

Embodiment 131. The method of embodiment 130, wherein the polypeptide capable of binding clusterin is selected from an antibody, a single-domain antibody, a heavy-chain-only antibody (VHH), a single-chain antibody (scFv), or a binding fragment thereof.

Embodiment 132. The method of any one of embodiments 126 to 131, wherein the HIC media is selected from MACRO-PREP METHYL, MACRO-PREP T-BUTYL (BIO-RAD), CAPTO PHENYL, CAPTO BUTYL (GE Healthcare), TOYOPEARL HIC (Tosoh), FRACTOGEL EMD PHENYL (Merck Millipore) and a membrane adsorber (e.g. SARTOBIND HIC (Sartorius)).

Embodiment 133. The method of any one of embodiments 126 to 132, wherein the solid support is a bead, a chromatography resin or a membrane.

Embodiment 134. The method of embodiment 133, wherein the bead is an agarose bead.

Embodiment 135. The method of embodiment 134, wherein the agarose bead is an aldehyde-activated agarose bead.

Embodiment 136. The method of any one of embodiments 133 to 135, wherein the immobilized polypeptide is coupled to the bead via a free NH₂.

Embodiment 137. The method of embodiment 133, wherein the chromatography resin comprises crosslinked poly[styrene divinylbenzene].

Embodiment 138. The method of any one of embodiments 133 to 137, wherein the HIC media that is conjugated to the third solid support is located in a fourth chromatography column or a membrane.

Embodiment 139. The method of embodiment 138, wherein the fourth chromatography column or the membrane is washed with a buffer.

Embodiment 140. The method of embodiment 139, wherein the comprises an ingredient selected from a salt, a detergent, an alcohol, a buffering agent and a combination thereof.

Embodiment 141. The method of embodiment 140, wherein the buffer is ice cold.

Embodiment 142. The method of any one of embodiments 139 to 141, wherein the washing step the fourth chromatography column removes the chimeric protein and/or the at least one HCP that is indirectly attached to the third solid support.

Embodiment 143. The method of any one of embodiments 133 to 142, further comprising contacting the chimeric protein that is directly attached to the third solid support with a first elution solution that releases the chimeric protein from the third chromatography column, thereby forming a third eluate which comprises the chimeric protein.

Embodiment 144. The method of any one of embodiments 133 to 143, wherein the hydrophobic interaction chromatography removes at least one HCP.

Embodiment 145. The method of embodiment 143, wherein the third eluate comprises fewer contaminants than the solution comprising the chimeric protein that was contacted with the HIC media that is conjugated to a third solid support.

Embodiment 146. The method of embodiment 145, wherein the contaminants comprise components of a mammalian cell that harbors a nucleic acid that expresses the chimeric protein.

Embodiment 147. A method for isolating and/or purifying a chimeric protein from a solution, wherein the chimeric protein has the structure selected from:
X-Linker-Y,
   X-Linker
      and
   Linker-Y
wherein the X and the Y are independently selected from a mammalian secreted protein, mammalian intracellular protein, a mammalian membrane protein, or a fragment thereof, the method comprises:
(i) providing a solution comprising the chimeric protein, and performing:
(a) a clusterin-scavenging chromatography,
(b) an affinity chromatography,
(c) an ion exchange chromatography, and/or
(d) a hydrophobic interaction chromatography, and thereby isolating and/or purifying the chimeric protein.

Embodiment 148. The method of embodiment 147, wherein the clusterin-scavenging chromatography comprises:
(a) providing a solution comprising the chimeric protein, the solution further comprising one or more host cell proteins (HCP),
(b) contacting the solution comprising the chimeric protein with a polypeptide immobilized onto the surface of a solid support, wherein the immobilized polypeptide is capable of binding clusterin, and
(c) recovering the protein that does not bind to the polypeptide immobilized onto the surface of the solid support.

Embodiment 149 The method of embodiment 147 or embodiment 148, wherein the affinity chromatography uses a moiety having affinity for the X, the Y, the Fc domain and/or the Fab domain.

Embodiment 150. The method of embodiment 149, wherein the moiety having affinity for the Fc domain is selected from protein A, a protein G, protein L, and protein M.

Embodiment 151. The method of any one of embodiments 147 to 150, wherein the ion exchange chromatography is selected from cation exchange chromatography and anion exchange chromatography.

Embodiment 152 A method for isolating and/or purifying a chimeric protein from a solution, wherein the chimeric protein has the structure selected from:
X-Linker-Y,
   X-Linker
      and
   Linker-Y
wherein the X and the Y are independently selected from a mammalian secreted protein, mammalian intracellular protein, a mammalian membrane protein, or a fragment thereof, the method comprises:
(i) performing a clusterin-scavenging chromatography comprising:
(a) providing a solution comprising the chimeric protein, the solution further comprising one or more host cell proteins (HCP),
(b) contacting the solution comprising the chimeric protein with a polypeptide immobilized onto the surface of a solid support, wherein the immobilized polypeptide is capable of binding clusterin, and
(c) recovering the protein that does not bind to the polypeptide immobilized onto the surface of the solid support,
(ii) performing an affinity chromatography selected from FcXL chromatography, protein A chromatography, a protein G chromatography, protein L chromatography, and protein M chromatography,
(iii) performing an ion exchange chromatography selected from cation exchange chromatography and anion exchange chromatography, and/or
(iv) performing a hydrophobic interaction chromatography, and thereby isolating and/or purifying the chimeric protein.

Embodiment 153. The method of any one of embodiments 147 to 152, wherein the clusterin-scavenging chromatography follows one or more of the affinity chromatography, ion exchange chromatography and hydrophobic interaction chromatography.

Embodiment 154. The method of any one of embodiments 147 to 152, wherein the clusterin-scavenging chromatography precedes one or more of the affinity chromatography, ion exchange chromatography and hydrophobic interaction chromatography.

Embodiment 155. The method of any one of embodiments 1 to 66, or 147 to 154, wherein the method removes clusterin and at least one more HCP.

Embodiment 156. The method of embodiment 155, wherein the at least one more HCP is selected from glyceraldehyde-3-phosphate dehydrogenase (GAPDH), actin (e.g., actin, cytoplasmic 2 isoform X2), and serine protease HTRA1 isoform X2 (HTRA).

Embodiment 157. The method of any one of embodiments 1 to 155, wherein the method removes at least by 30%, or at least 40%, or at least 40%, or at least 50%, or at least 60%, or at least 70%, or at least 80%, or at least 90% of clusterin compared to the solution comprising the chimeric protein.

Embodiment 158. The method of any one of embodiments 155 to 157, wherein the method removes at least by 10%, or at least 20%, or at least 30%, or at least 40%, or at least 50%, or at least 60%, or at least 70% of the at least one more HCP compared to the solution comprising the chimeric protein.

Embodiment 159. An isolated and/or purified chimeric protein prepared using the method of any one of embodiments 1 to 158.

Embodiment 160. A composition comprising the isolated and/or purified chimeric protein of embodiment 159.

Embodiment 161. A composition comprising an isolated and/or purified chimeric protein prepared using the method of any one of embodiments 1 to 158.

Embodiment 162. A pharmaceutical composition comprising the isolated and/or purified chimeric protein of embodiment 149, and a pharmaceutically acceptable excipient.

Embodiment 163. A pharmaceutical composition comprising an isolated and/or purified chimeric protein prepared using the method of any one of embodiments 1 to 158, and a pharmaceutically acceptable excipient.

Embodiment 164. A method for treating a cancer or an autoimmune disease in a subject in need thereof, the method comprising administering to the subject a therapeutically-effective amount of an isolated and/or purified chimeric protein prepared using the method of any one of embodiments 1 to 158.

Embodiment 165. A method for treating a cancer or an autoimmune disease in a subject in need thereof, the method comprising administering to the subject a therapeutically-effective amount of the isolated and/or purified chimeric protein of embodiment 159.

Embodiment 166. A method for treating a cancer or an autoimmune disease in a subject in need thereof, the method comprising administering to the subject a therapeutically-effective amount of the composition of embodiment 160 or embodiment 161.

Embodiment 167. A method for treating a cancer or an autoimmune disease in a subject in need thereof, the method comprising administering to the subject a therapeutically-effective amount the pharmaceutical composition of embodiment 162 or embodiment 163.

Embodiment 168. A method for treating a cancer or an autoimmune disease in a subject in need thereof, the method comprising administering to the subject a therapeutically-effective amount of a chimeric protein having the structure selected from:
X-Linker
   and
Linker-Y
wherein the X and the Y is selected from a mammalian secreted protein, mammalian intracellular protein, a mammalian membrane protein, an antigen, or a fragment thereof, and
the linker comprises an Fc domain,
wherein the chimeric protein is isolated and/or purified chimeric protein prepared using the method of any one of embodiments 1 to 158.

Embodiment 169. A method for treating a cancer or an autoimmune disease in a subject in need thereof, the method comprising administering to the subject a therapeutically-effective amount of a chimeric protein having the structure:
X-Linker-Y
wherein X and/or Y is selected from a mammalian secreted protein, mammalian intracellular protein, a mammalian membrane protein, an antigen, or a fragment thereof, and
the linker comprises an Fc domain,
wherein the chimeric protein is isolated and/or purified chimeric protein prepared using the method of any one of embodiments 1 to 158.

170. A method for isolating and/or purifying a protein comprising an Fc domain and/or a Fab domain, the method comprising, in the following order:
(a) providing a solution comprising the protein comprising the Fc domain and/or the Fab domain, the solution further comprising one or more host cell proteins (HCPs),
(b) contacting the solution comprising the protein comprising the Fc domain and/or the Fab domain with a polypeptide capable of binding clusterin to produce a mixture, optionally wherein the polypeptide capable of binding clusterin is conjugated to a moiety selected from a magnetic bead, a tag, and combination thereof, and
(c) removing free clusterin, clusterin bound to the polypeptide, and/or any additional HCPs contemporaneously bound to clusterin and/or the polypeptide from the mixture, and thereby isolating and/or purifying the protein comprising the Fc domain and/or the Fab domain.

171. The method of embodiment 170, wherein the polypeptide capable of binding clusterin is conjugated to a magnetic bead and the step (c) comprises, in the following order:
(i) contacting the mixture with a magnetic field, and
(ii) recovering a second solution comprising the protein comprising the Fc domain and/or the Fab domain, the second solution being substantially free of the polypeptide capable of binding clusterin and/or the magnetic bead.

172. The method of embodiment 170, wherein the polypeptide capable of binding clusterin is conjugated to a tag and the and the step (c) comprises, in the following order:
(i) contacting the solution with a tag-binding agent, optionally wherein the tag-binding agent is immobilized on a solid support, and
(ii) recovering a second solution comprising the protein comprising the Fc domain and/or the Fab domain, the second solution being substantially free of the polypeptide capable of binding clusterin and/or the tag.

173. A method for isolating and/or purifying a protein comprising an Fc domain and/or a Fab domain, the method comprising, in the following order:
(a) providing a solution comprising the protein comprising the Fc domain and/or the Fab domain, the solution further comprising one or more host cell proteins (HCP),
(b) contacting the solution comprising the protein comprising the Fc domain and/or the Fab domain with a polypeptide immobilized onto the surface of a solid support, wherein the immobilized polypeptide is capable of binding clusterin, and
(c) removing free clusterin, clusterin bound to the polypeptide, and any additional HCPs contemporaneously bound to clusterin and/or the polypeptide from the solution, thereby isolating and/or purifying the protein comprising the Fc domain and/or the Fab domain.

174. The method of any one of embodiments 170 to 173, wherein the protein comprising the Fc domain and/or the Fab domain is a fusion protein.

175. The method of embodiment 174, wherein the fusion protein comprises the formula:
(i) X-Fc, wherein X is selected from an antigen, a mammalian intracellular protein, a mammalian membrane protein, a mammalian secreted protein or a fragment thereof;
(ii) Fc-Y, wherein Y is selected from an antigen, a mammalian intracellular protein, a mammalian membrane protein, a mammalian secreted protein or a fragment thereof; or
(iii) X-Fc-Y, wherein X and Y are independently selected from an antigen, a mammalian intracellular protein, a mammalian membrane protein, a mammalian secreted protein or a fragment thereof.

176. A method for isolating and/or purifying a protein comprising an Fc domain and/or a Fab domain, the method comprises:
(i) providing a solution comprising the protein comprising the Fc domain and/or the Fab domain, and performing, in the following order:
(a) a clusterin-scavenging chromatography,
(b) an affinity chromatography,
(c) an ion exchange chromatography, and/or
(d) a hydrophobic interaction chromatography, and thereby isolating and/or purifying the protein comprising the Fc domain and/or the Fab domain.

177. The method of embodiment 176, wherein the clusterin-scavenging chromatography comprises, in the following order:
(a) providing a solution comprising the protein comprising the Fc domain and/or the Fab domain, the solution further comprising one or more host cell proteins (HCP),
(b) contacting the solution comprising the protein comprising the Fc domain and/or the Fab domain with a polypeptide immobilized onto the surface of a solid support, wherein the immobilized polypeptide is capable of binding clusterin, and
(c) recovering the protein that does not bind to the polypeptide immobilized onto the surface of the solid support.

178. The method of embodiment 176 or embodiment 177, wherein the affinity chromatography uses a moiety having affinity for the X, the Y, the Fc domain and/or the Fab domain.

179. The method of embodiment 178, wherein the moiety having affinity for the Fc domain is selected from protein A, a protein G, protein L, and protein M.

180. The method of any one of embodiments 176 to 179, wherein the ion exchange chromatography is selected from cation exchange chromatography and anion exchange chromatography.

181. A method for isolating and/or purifying a protein comprising an Fc domain and/or a Fab domain, the method comprising, in the following order:
(i) performing a clusterin-scavenging chromatography comprising:
   (a) providing a solution comprising the protein comprising the Fc domain and/or the Fab domain, the solution further comprising one or more host cell proteins (HCP),
   (b) contacting the solution comprising the protein comprising the Fc domain and/or the Fab domain with a polypeptide immobilized onto the surface of a solid support, wherein the immobilized polypeptide is capable of binding clusterin, and
   (c) recovering the protein that does not bind to the polypeptide immobilized onto the surface of the solid support,
(ii) performing an affinity chromatography selected from FcXL chromatography, protein A chromatography, a protein G chromatography, protein L chromatography, and protein M chromatography,
(iii) performing an ion exchange chromatography selected from cation exchange chromatography and anion exchange chromatography, and/or
(iv) performing a hydrophobic interaction chromatography, and thereby isolating and/or purifying the protein comprising the Fc domain and/or the Fab domain.

### EXAMPLES

The examples herein are provided to illustrate advantages and benefits of the present disclosure and to further assist a person of ordinary skill in the art with preparing Fc domain-containing chimeric proteins. The examples herein are also presented in order to more fully illustrate the preferred aspects of the present disclosure. The examples should in no way be construed as limiting the scope of the present disclosure, as defined by the appended claims. The examples can include or incorporate any of the variations, aspects or embodiments of the present disclosure described above. The variations, aspects or embodiments described above may also further each include or incorporate the variations of any or all other variations, aspects or embodiments of the present disclosure.

### Example 1: The Individual CHO Host Cell Proteins (HCPs) that Form Contaminants in Preparations of a Protein Containing an Fc Domain Purified Using Conventional Methods

Purification of proteins containing Fc domains (e.g., antibodies and Fc fusion proteins) from mammalian cell expression systems typically involves growth of transfected mammalian cells (e.g., Chinese hamster ovary (CHO) cells and derivatives) in large bioreactors (FIG. 1). The cells are harvested by centrifugation and/or filtration to yield a clarified supernatant (FIG. 1). The fusion protein is then substantially enriched with an affinity chromatography resin, such as protein A or FcXL chromatography, before polishing with additional chromatographic methods. The typical additional chromatographic methods are ion exchange chromatography and hydrophobic interaction chromatography. See, e.g., McCue et al., Manufacturing process used to produce long-acting recombinant factor VIII Fc fusion protein, Biologicals 43(4): 213-219 (2015).

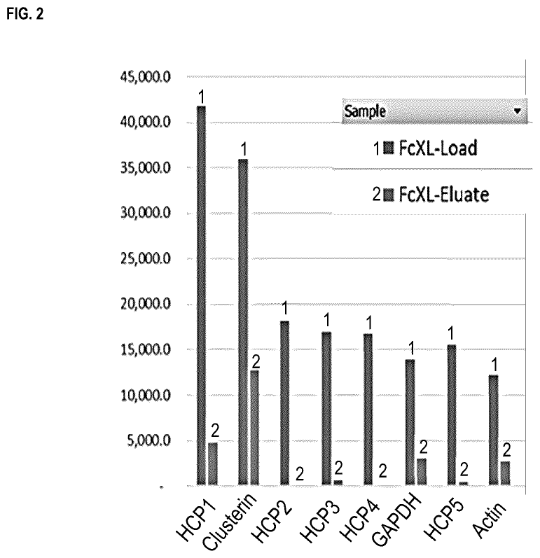

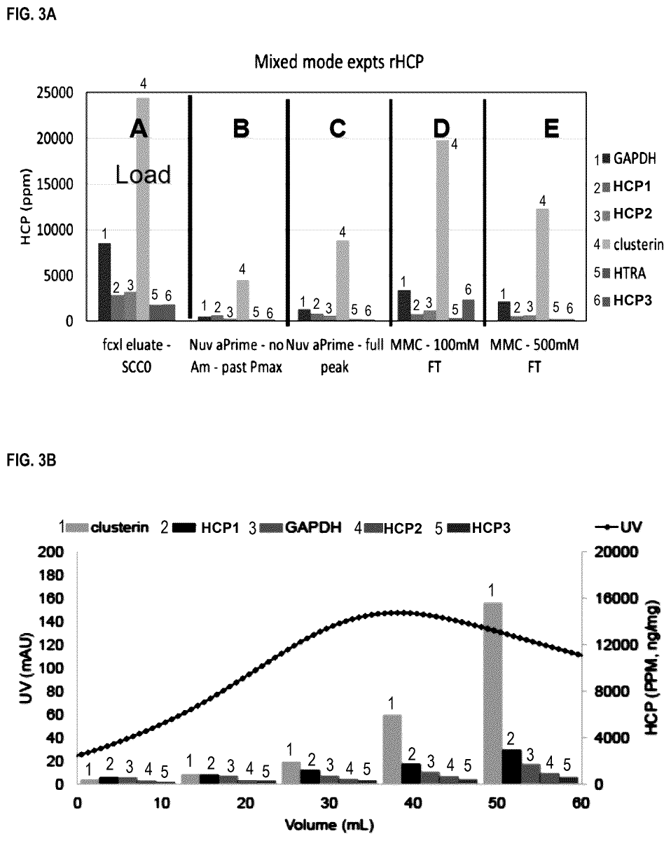

To purify a protein containing an Fc domain a conventional scheme was developed for the purification of a chimeric protein containing Fc domain was studied. In brief, CHO cells were transfected with a DNA construct encoding the protein containing an Fc domain having a secretion signal. Cells were grown in a large reactor (FIG. 1). A clarified harvest was generated from the culture supernatant by filtration of intact cells, organelles and other large protein complexes, which was used as the starting material for the purification of the fusion protein. The clarified harvest was subjected to affinity chromatography using FcXL resin. This resin captures Fc domain of the protein and thereby purifies the protein. The protein eluate of the FcXL chromatography contained the fusion protein. However, the fusion protein was contaminated with a number of host cell proteins (HCPs) (data not shown). The eluate of the FcXL chromatography was polished using one or more of ion exchange chromatography, hydrophobic interaction chromatography (HIC), mixed mode chromatography, and ultrafiltration for HCP removal (see FIG. 3A and FIG. 3B and data not shown). However, these steps did not sufficiently remove the contaminating proteins. Therefore, a systematic identification of individual CHO host cell proteins that contaminate the protein was undertaken by mass spectrometry. Mass spectrometry was performed before and after every chromatography step to enable a granular understanding of which HCPs are cleared by each chromatography step. As shown in FIG. 2, 1-3 individual CHO host cell proteins (HCPs) account for much of the total HCP load in a purified sample of the protein containing an Fc domain. Clusterin was found to be a main protein that was consistently found to be a contaminant. Table 1 shows a list of the most abundant HCPs that contaminated the protein following FcXL chromatography in an exemplary experiment. As shown, 10,629.7 ppm clusterin was present in the purified protein preparation. Yeast alcohol dehydrogenase 1 was a positive control protein in the assay.

TABLE 1
Proteomic analysis of PDB00536 - FcXL chromatography eluates E1 +
E2 pooled. Yeast Alcohol dehydrogenase was used as an internal control.

| | MW | Top3-XIC- | PPM, | PPM, |
|---|---|---|---|---|
| Protein name | [Da] | AUC | moles | ng/mg |
| sp\|P00330\|ADH1_YEAST Alcohol dehydrogenase 1 | 36,713 | 5.57E+06 | 1,116.90 | 465.7 |
| OS = Saccharomyces cerevisiae (strain ATCC 204508/ | | | | |
| S288c) GN = ADH1 PE = 1 SV = 5 | | | | |
| XP_027246101.1 clusterin [Cricetulus griseus] | 51,747 | 9.02E+07 | 18,087.40 | 10,629.70 |
| NP_001231783.1 glyceraldehyde-3-phosphate | 35,744 | 6.10E+07 | 12,232.10 | 4,965.40 |
| dehydrogenase [Cricetulus griseus] | | | | |
| HCP1 | 129,679 | 4.59E+07 | 9,204.10 | 13,555.20 |
| XP_027281226.1 actin, cytoplasmic 2 isoform X2 | 41,787 | 4.25E+07 | 8,522.30 | 4,044.40 |
| [Cricetulus griseus] | | | | |
| HCP2 | 49,979 | 1.54E+07 | 3,088.10 | 1,752.80 |
| HCP3 | 57,883 | 1.50E+07 | 3,007.90 | 1,977.30 |
| HCP4 | 27,217 | 1.05E+07 | 2,105.50 | 650.8 |
| XP_027260200.1 serine protease HTRA1 isoform X2 | 51,257 | 8.71E+06 | 1,746.60 | 1,016.70 |
| [Cricetulus griseus] | | | | |

### Example 2: Attempts of Using Mixed-Mode Chromatography to Remove Contaminants from the Protein Containing an Fc Domain

The mass spectrometry data showed that clusterin, glyceraldehyde-3-phosphate dehydrogenase (GAPDH), actin, and serine protease HTRA1 isoform X2 (HTRA) were among the most common contaminating HCPs (See Table 1). Therefore, to remove these proteins, a number of other chromatography resins, including mixed mode chromatography, was used for further purification. To assess the effectiveness of these steps, the levels of clusterin, GAPDH, HTRA and three other HCPs (indicated in FIG. 3A as HCP1, HCP2 and HCP3) were measured before and after each purification step. The FcXL chromatography eluate, which was used as the input for mixed mode chromatography, contained clusterin (>22,500 ppm), GAPDH, HTRA, HCP1, HCP2 and HCP3 proteins at the levels shown in FIG. 3A (panel A).

As one example, Nuvia aPrime 4A hydrophobic anion exchange chromatography was used in an attempt to remove the HCP contaminants. The FcXL chromatography eluate was loaded on a Nuvia aPrime 4A hydrophobic anion exchange, the column was washed and the eluate was fractionated either after the maximum absorbance (peak max, Pmax) and including the full peak, and both fractions were analyzed for the presence of clusterin, GAPDH, HTRA, HCP1, HCP2 and HCP3 proteins. Although, the levels of the contaminants decreased, the eluates after Pmax (FIG. 3A, Panel B), and at full peak (FIG. 3A, Panel C) both contained each of clusterin, GAPDH, HTRA, HCP1, HCP2 and HCP3 proteins. Specifically, the eluates after Pmax and at full peak contained >4,000 ppm clusterin (FIG. 3A, Panel B), and >7,500 ppm clusterin (FIG. 3A, Panel C), respectively.

In another example, CAPTO MMC multimodal weak cation exchange chromatography was used as another technique in an attempt to remove the contaminants. The FcXL chromatography eluate was loaded on a CAPTO MMC multimodal weak cation exchange in the presence of 100 mM NaCl or 500 mM NaCl, and flow through samples were analyzed for the presence of clusterin, GAPDH, HTRA, HCP1, HCP2 and HCP3 proteins. Although, the levels of the contaminants decreased to a smaller extent, flow through in the presence of 100 mM NaCl (FIG. 3A, Panel D), and 500 mM NaCl (FIG. 3A, Panel E) each of clusterin, GAPDH, HTRA, HCP1, HCP2 and HCP3 proteins. Specifically, the flow through in the presence of 100 mM NaCl and 500 mM NaCl contained about 20,000 ppm (FIG. 3A, Panel D), and about 12,000 ppm clusterin (FIG. 3A, Panel E).

These results indicate, inter alia, that neither Nuvia aPrime 4A hydrophobic anion exchange chromatography or CAPTO MMC multimodal weak cation exchange chromatography were successful in removal of clusterin. Nonetheless, these data suggest, inter alia, that if some of the contaminants can be removed by another method, Nuvia aPrime 4A hydrophobic anion exchange chromatography or CAPTO MMC multimodal weak cation exchange chromatography could be considered as one of the steps for purification of the protein containing an Fc domain.

To further explore purification, clusterin, GAPDH, and three other HCPs (indicated as HCP1, HCP2 and HCP3 in FIG. 3B) were monitored during anion exchange chromatography. In brief, the FcXL chromatography eluate was loaded on an anion exchange column and fractions were collected. The total protein was measured by absorbance at 280 nm and plotted as a function of retention volume. As shown in FIG. 3B, a broad protein peak was observed with a peak elution (retention) volume of about 40 ml. Clusterin, GAPDH, HCP1, HCP2 and HCP3 were measured in peaks corresponding to 10, 20, 30, 40 and 50 ml of retention volume. As shown in FIG. 3B, the relative abundance of clusterin (and other proteins) increased with increasing retention volume. The samples at 50 ml were after total protein peak, contained more clusterin compared to the 40 ml samples, where a peak of total protein was seen (FIG. 3B). These data confirmed that the concentration of clusterin in a chromatography eluate correlated with the concentration of the desired Fc containing fusion protein, and that clusterin was a particularly problematic HCP to remove from the clarified harvest. These data suggest, inter alia, that the protein containing an Fc domain complex and clusterin-containing protein complex may form two distinct complexes. However, the peaks corresponding to the complexes showed a significant overlap.

Purification of other Fc domain containing proteins containing different fusion partners was studied using FcXL chromatography. It was observed that clusterin was a major contaminant in other proteins as well (data not shown and see Example 5 infra). Many other HCPs were also observed to contaminate these preparations (data not shown).

Since the clusterin-containing protein complex could not be separated using all of the chromatography techniques discussed herein, these data collectively indicate, inter alia, that clusterin binds Fc domain of the protein containing an Fc domain. this is consistent with previous reports. See Wilson and Easterbrook-Smith, Clusterin binds by a multivalent mechanism to the Fc and Fab regions of IgG, Biochim Biophys Acta 1159(3): 319-26 (1992); see Example 7 infra. These data also may collectively indicate, inter alia, that other HCPs found in the protein containing an Fc domain preparation also may form similar complexes with each other and/or with the proteins containing an Fc domains.

### Example 3: Production of a Clusterin-Scavenger Resin

To prepare an HCP-scavenging affinity resin, llama antibodies were generated. In brief, llamas were immunized with CHO host cell proteins (FIG. 4A). Antibody clones specific to clusterin were purified (FIG. 4A). Clones that made highly specific and high-affinity VHH that bound clusterin at epitopes selected to be non-competitive for the epitopes required for binding of clusterin to antibody-derived Fc and/or Fab domains (FIG. 4A). Two top prototype VHH domain leads were loaded them onto POROS or agarose resins (FIG. 4B). These are also referred to herein as clusterin scavenger resin or clusterin scavenging resin.

### Example 4: The Clusterin-Scavenger Resin Removed Clusterin and Other Proteins from the FcXL Eluate

As illustrated in FIG. 4B, it was hypothesized that if a preparation of the protein containing an Fc domain that contains clusterin and other contaminants (such as the FcXL eluate) is loaded on a column containing the clusterin scavenger resin, clusterin and any protein containing an Fc domain that is bound to clusterin would be retained on the column and pure protein containing an Fc domain would come out in the flow through. To test this hypothesis, chromatography with clusterin-scavenger resin was performed (FIG. 5A). In brief, an FcXL chromatography eluate was loaded on a column containing clusterin scavenger resin. Flow through was collected. The protein bound to the clusterin-scavenging chromatography column was eluted using a strip buffer (e.g., an acidic buffer such as glycine at pH 3.5). The amounts of GAPDH, actin, HTRA1, clusterin four other HCPs (identified as HCP1, HCP2, HCP3 and HCP4 in FIG. 5B) were measured in the load (FcXL chromatography eluate), flow-through and the eluate. As expected, the load contained about 6000 ppm of total HCP, including about 3000 ppm of clusterin (FIG. 5B). The flow through showed a substantially less clusterin compared to the load, as expected for a clusterin-scavenging resin (FIG. 5C). GAPDH, actin, and HTRA1, HCP1, HCP2, HCP3 and HCP4 were not expected to bind the clusterin-scavenging chromatography column, and it was expected that the abundance of these proteins would not change following clusterin-scavenging chromatography. However, surprisingly, as shown in FIG. 5B, the amounts of many other HCPs that were tested (GAPDH, actin, HTRA1, HCP1, HCP2, HCP3 and HCP4) were also decreased in the flow through compared to compared to the load. All of the proteins were retained by the clusterin scavenger resin were stripped and analyzed. As shown in FIG. 5B, in addition to clusterin, many other HCPs that were tested (GAPDH, actin, HTRA1, HCP1, HCP2, HCP3 and HCP4) were also found in the eluate.

To further gain a further understanding of the efficacy of removal of HCP by the clusterin-scavenging chromatography a proteomic analysis of the flow through of the clusterin-scavenging chromatography and the eluate of the clusterin-scavenging chromatography was performed. The same FcXL chromatography eluate (load) as analyzed in Table 1 was loaded on a clusterin-scavenging chromatography column and flow through fraction was subjected to proteomic analysis using mass spectrometry. Those data are shown below in Table 2. The level of clusterin decreased from 10,629.7 ppm to 3,485.3 ppm after a clusterin-scavenging chromatography run (compare Table 1 and Table 2). Likewise, many other HCPs also showed a large decrease after a clusterin-scavenging chromatography run. The protein bound to the clusterin-scavenging chromatography column was eluted using a strip buffer, and the eluate fraction was subjected to proteomic analysis using mass spectrometry. Those data are shown below in Table 3. The proteins that decreased in amount in the flow through after a clusterin-scavenging chromatography run, were present in the eluate.

These data demonstrate that clusterin binds Fc domain and forms one or more complexes that further contain one or more of the other HCPs. Surprisingly, therefore, a clusterin-scavenging chromatography removes a variety of HCPs that contaminate the preparations of the proteins containing an Fc domain that were not specifically targeted for removal by the clusterin-specific scavenging resin.

TABLE 2
Proteomic analysis of the flow through from clusterin scavenger chromatography.
Yeast Alcohol dehydrogenase was used as an internal control.

| | MW | Top3-XIC- | PPM, | PPM, |
|---|---|---|---|---|
| Protein name | [Da] | AUC | moles | ng/mg |
| sp\|P00330\|ADH1_YEAST Alcohol dehydrogenase 1 | 36,713 | 5.86E+06 | 1,166.20 | 486.2 |
| OS = Saccharomyces cerevisiae (strain ATCC 204508/ | | | | |
| S288c) GN = ADH1 PE = 1 SV = 5 | | | | |
| NP_001231783.1 glyceraldehyde-3-phosphate | 35,744 | 4.45E+07 | 8,856.10 | 3,595.00 |
| dehydrogenase [Cricetulus griseus] | | | | |
| XP_027246101.1 clusterin [Cricetulus griseus] | 51,747 | 2.98E+07 | 5,930.60 | 3,485.30 |
| XP_027281226.1 actin, cytoplasmic 2 isoform X2 | 41,787 | 2.96E+07 | 5,890.80 | 2,795.50 |
| [Cricetulus griseus] | | | | |
| HCP1 | 129,679 | 2.90E+07 | 5,771.40 | 8,499.70 |
| HCP2 | 49,979 | 1.29E+07 | 2,567.30 | 1,457.20 |
| HCP3 | 57,883 | 1.13E+07 | 2,248.90 | 1,478.30 |
| HCP4 | 27,217 | 8.70E+06 | 1,731.40 | 535.2 |
| HCP5 | 14,797 | 8.12E+06 | 1,616.00 | 271.6 |
| HCP6 | 17,897 | 6.68E+06 | 1,329.40 | 270.2 |
| HCP7 | 48,240 | 6.38E+06 | 1,269.70 | 695.6 |
| XP_027260200.1 serine protease HTRA1 isoform X2 | 51,257 | 6.09E+06 | 1,212.00 | 705.5 |
| [Cricetulus griseus] | | | | |

TABLE 3
Proteomic analysis of the eluate from clusterin scavenger chromatography.
Yeast Alcohol dehydrogenase was used as an internal control.

| | MW | Top3-XIC- | PPM, | PPM, |
|---|---|---|---|---|
| Protein name | [Da] | AUC | moles | ng/mg |
| sp\|P00330\|ADH1_YEAST Alcohol dehydrogenase 1 | 36,713 | 5.85E+06 | 1,124.90 | 469 |
| OS = Saccharomyces cerevisiae (strain ATCC 204508/S288c) | | | | |
| GN = ADH1 PE = 1 SV = 5 | | | | |
| XP_027246101.1 clusterin [Cricetulus griseus] | 51,747 | 6.78E+08 | 130,370.10 | 76,616.20 |
| NP_001231783.1 glyceraldehyde-3-phosphate dehydrogenase | 35,744 | 1.66E+08 | 31,919.50 | 12,957.20 |
| [Cricetulus griseus] | | | | |
| XP_027281226.1 actin, cytoplasmic 2 isoform X2 | 41,787 | 1.06E+08 | 20,382.30 | 9,672.60 |
| [Cricetulus griseus] | | | | |
| HCP1 | 129,679 | 6.33E+07 | 12,171.70 | 17,925.70 |
| XP_027260200.1 serine protease HTRA1 isoform X2 | 51,257 | 2.88E+07 | 5,537.80 | 3,223.60 |
| [Cricetulus griseus] | | | | |

Several clusterin-scavenging chromatography runs were performed to purify an Fc domain containing protein, the SIRPα-Fc-CD40L chimeric protein, using different resin loading parameters, two different llama anti-clusterin VHH (1765 and 1762) that are non-competitive for the binding of clusterin to Fc and/or Fab domains of antibody. In this experiment, the amount of HOP decreased clusterin by a factor of 3-4 (data not shown). When clusterin-scavenging chromatography was combined with other chromatographic steps to clusterin, could be decreased to 300 to 400 ppm (data not shown).

To further evaluate the efficiency of the clusterin-scavenging chromatography in removing HOPs from a preparation of an Fc domain containing protein, many HOPS were followed during a purification process. Specifically, FcXL eluate was loaded onto a clusterin-scavenging resin packed at 50 g/L, 100 g/L or 150 g/L density. FcXL eluate, flow though from the 50 g/L clusterin-scavenging chromatography resin (“1765-P10 50 g/L”), flow though from the 100 g/L clusterin-scavenging chromatography resin (“1765-P10 100 g/L”), wash (“1765-P10 W”) and eluate using a “strip” buffer (“1765-P10 S”) were used for assaying clusterin, GAPDH, and three other HOPs (identified as HCP1, HCP2 and HCP3 in FIG. 5D). As shown in FIG. 5D, each of clusterin, GAPDH, HCP1, HCP2 and HCP3 decreased in the flow though from the clusterin-scavenging chromatography compared to the FcXL eluate irrespective of the density of the clusterin-scavenging chromatography resin. However, it was observed that the clusterin-scavenging chromatography resin packed at 50 g/L density was more efficient in removal of each of clusterin, GAPDH, HCP1, HCP2 and HCP3. All proteins became enriched in the eluate using a “strip” buffer (FIG. 5D). The amount of clusterin removed by the clusterin-scavenging resin also depended on density, and the clusterin-scavenging chromatography resin packed at 50 g/L density was more efficient in removal of clusterin (FIG. 5E). These results illustrate, inter alia, that efficiency of clusterin-scavenger chromatography may be improved by changing chromatography conditions.

Based on these data, several exemplary purification schemes were compared for the purification of an Fc domain containing protein (FIG. 5F). Three different purification schemes were tested. The first scheme (indicated as Process 1 in FIG. 5F) included FcXL chromatography, anion exchange chromatography, affinity capture chromatography, and hydrophobic interaction chromatography (HIC). The second scheme (indicated as Process 2 in FIG. 5F) included FcXL chromatography, anion exchange chromatography, hydrophobic interaction chromatography (HIC) and filtration. The third scheme (indicated as Process 3 in FIG. 5F) included FcXP chromatography, hydrophobic interaction chromatography (HIC), clusterin-scavenging chromatography, and ion exchange chromatography. As shown in FIG. 5F, the purification process that uses the clusterin-scavenging chromatography exhibited an increase in the overall process yield compared to other polishing chromatography steps attempted. These results indicate, inter alia, that (1) the overall process yield is unpredictable, and (2) a purification scheme comprising clusterin-scavenging chromatography is very efficient in removing HCPs and achieving high overall process yield.

### Example 5: Purification of a Different Fc Domain Containing Protein Using the Clusterin-Scavenger Resin and Other Chromatography Steps

Purification of a different Fc domain containing protein containing different fusion partners, the CSF1R-Fc-CD40L chimeric protein, was studied using FcXL chromatography. The protein was purified using FcXL chromatography. Three different batches FcXL chromatography eluates were loaded onto the clusterin-scavenger resin for removal of clusterin. Total HCP content was analyzed in the three batches of FcXL chromatography eluates before and after clusterin-scavenger chromatography. As shown in FIG. 6A, the FcXL chromatography eluates contained about 13500 to 15000 ppm of total HCPs. After clusterin-scavenger chromatography (ACR), HCPs decreased to about 2000 to 2500 ppm, indicating over six-fold purification (FIG. 6A). These results illustrate, inter alia, that yield of clusterin-scavenger chromatography may be improved by changing chromatography conditions.

Purification of the CSF1R-Fc-CD40L chimeric protein, which is an Fc domain containing protein, was studied using FcXL chromatography, the clusterin-scavenger chromatography (ACR), and two further polishing chromatography steps (shown as Polish 1 and Polish 2 in FIG. 6B). The eluate of FcXL chromatography, the flow through of ACR, the protein purified after Polish 1 and Polish 2 were analyzed to quantitate the amounts of clusterin, glyceraldehyde-3-phosphate dehydrogenase (GAPDH), actin, serine protease HTRA1 isoform X2 (HTRA), three other HCPs (identified in FIG. 6B as HCP1, HCP2 and HCP3). As shown in FIG. 6B, compared to the eluate of FcXL chromatography, the flow through of ACR showed decreased levels of each of the proteins tested. As expected, compared to the eluate of FcXL chromatography, the flow through of ACR showed a decreased amount of clusterin (FIG. 6B). However, surprisingly, the amounts of GAPDH, actin, HTRA, HCP1, HCP2 and HCP3, which were not expected to bind the clusterin-scavenger resin, also decreased in the flow through of ACR compared to the FcXL eluate. It was seen that each of the HCPs further decreased following Polish 1 and Polish 2 steps. These results indicate, inter alia, that Fc domain containing proteins may be purified using clusterin-scavenger chromatography in combination with other chromatography steps.

### Example 6: Study of Chromatography Parameters Using the Clusterin-Scavenger Resin

Chromatography parameters were studied for clusterin-scavenger chromatography. In one experiment, a clusterin-scavenger chromatography column was loaded with an eluate from FcXL chromatography, which contained 0.62 mg/mL total protein, including 16192 ppm (10039 ng/mL) of HCP. During this experiment, the following two parameters were varied: (1) conductivity: a high conductivity of 17.5 mS/cm (which matched the conductivity of equilibrium buffer) or low conductivity of 10 mS/cm were tested; and (2) a load rate: a high load rate of 25 g/L or a low load rate of 10 g/L were tested. The results obtained from this study are shown below in Table 4. As shown in Table 4, under the conditions used in this experiment, yield of clusterin-scavenger chromatography increased when conductivity and load rate were altered. However, overall, the yield under given conditions were unpredictable. Optimal conditions for one variable depended on other variables (data not shown).

TABLE 4
Effect of conductivity and load rate on yield

| | | Load | Pool | | | Yield |
|---|---|---|---|---|---|---|
| Sample | Load | Conductivity | Concentration | HCP | HCP | (fold |
| ID | Rate (g/L) | (mS/cm) | (mg/mL) | (ng/mL) | (ppm) | change) |
| ACR01 | 10 | 10.0 | 0.034 | 896 | 26719 | 1× |
| ACR02 | 25 | 17.5 | 0.224 | 3278 | 14653 | 4.5× |
| ACR03 | 25 | 10.0 | 0.184 | 4552 | 24717 | 5× |
| ACR04 | 25 | 25.0 | 0.327 | 4087 | 12490 | 6× |

These results illustrate, inter alia, that yield of clusterin-scavenger chromatography may be improved by changing chromatography conditions.

### Example 7: Chinese Hamster Clusterin Binds Human Fc Domain Containing Proteins

It has been previously reported that human clusterin binds human antibodies. See Wilson and Easterbrook-Smith, Clusterin binds by a multivalent mechanism to the Fc and Fab regions of IgG, Biochim Biophys Acta 1159(3): 319-26 (1992). To understand whether the Cricetulus griseus clusterin found in CHO cells binds to proteins containing human Fc domains, a Meso Scale Discovery (MSD)-ELISA-based method was used.

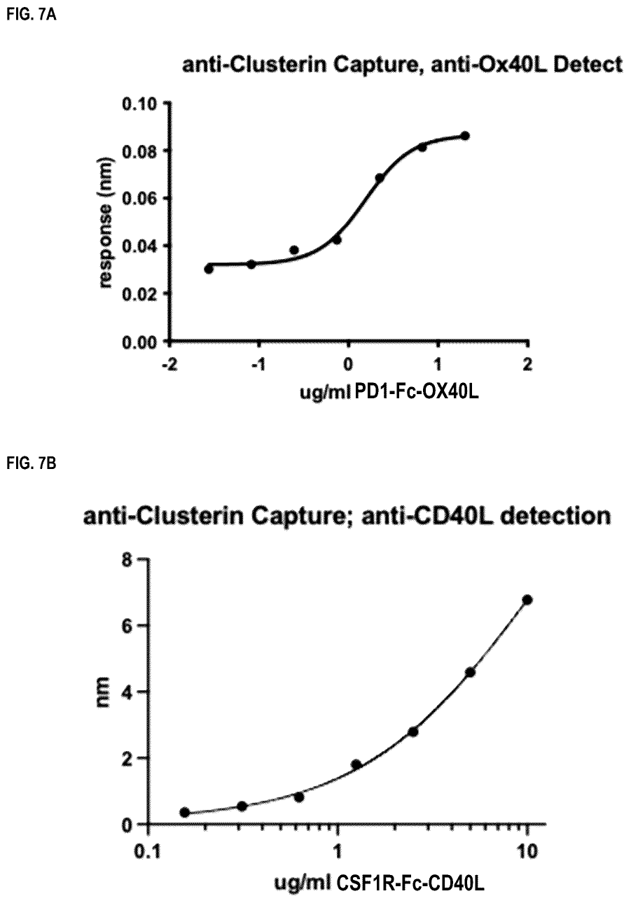

In one experiment, briefly, an anti-clusterin antibody was coated on a plate. Increasing amounts of FcXL eluate during the purification of a protein containing human Fc domain (the PD1-Fc-OX40L chimeric protein) was added to the plate for the capture by the plate bound anti-clusterin antibody. Any of the protein containing human Fc domain captured by the plate-bound anti-clusterin antibody was detected using an anti-human OX40L antibody and a SULFO-TAG conjugated secondary antibody. Since anti-clusterin antibody was used to capture, only the protein containing human Fc domain that is pre-associated with clusterin will generate a signal in this format. FIG. 7A shows a curve showing the binding. As shown in FIG. 7A, there was a dose-dependent and saturable response. These results indicate, inter alia, that Cricetulus griseus clusterin found in CHO cells binds to proteins containing human Fc domains.

In another experiment, briefly, an anti-clusterin antibody was coated on a plate. Increasing amounts of FcXL eluate during the purification of a second protein containing human Fc domain (the CSF1R-Fc-CD40L chimeric protein) was added to the plate for the capture by the plate bound anti-clusterin antibody. Any of the second protein containing human Fc domain captured by the plate-bound anti-clusterin antibody was detected using an anti-human CD40L antibody and a SULFO-TAG conjugated secondary antibody. Since anti-clusterin antibody was used to capture, only the protein containing human Fc domain that is pre-associated with clusterin will generate a signal in this format. FIG. 7B shows a curve showing the binding. As shown in FIG. 7B, there was a dose-dependent and saturable response. These results indicate, inter alia, that Cricetulus griseus clusterin found in CHO cells binds to proteins containing human Fc domains.

These results demonstrate, inter alia, that clusterin binds Fc-domain containing proteins and in part, explains the observed difficulty in the removal of clusterin from proteins containing Fc domains (e.g., antibodies and Fc fusion proteins) prepared in of transfected mammalian cells (e.g., Chinese hamster ovary (CHO) cells and derivatives).

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present disclosure is not entitled to antedate such publication by virtue of prior invention.

As used herein, all headings are simply for organization and are not intended to limit the disclosure in any manner. The content of any individual section may be equally applicable to all sections.

### EQUIVALENTS

While the invention has been disclosed in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

What is claimed is:

1. A method for isolating and/or purifying a protein comprising an Fc domain and/or a Fab domain, the method comprising:
(a) providing a solution comprising the protein comprising the Fc domain and/or the Fab domain, the solution further comprising one or more host cell proteins (HCPs),
(b) contacting the solution comprising the protein comprising the Fc domain and/or the Fab domain with a polypeptide capable of binding clusterin to produce a mixture, and
(c) removing free clusterin, clusterin bound to the polypeptide, and/or any additional HCPs contemporaneously bound to clusterin and/or the polypeptide from the mixture, and thereby isolating and/or purifying the protein comprising the Fc domain and/or the Fab domain.

2. The method of claim 1, wherein the polypeptide capable of binding clusterin is conjugated to a moiety selected from a magnetic bead, a tag, and combination thereof.

3. The method of claim 2, wherein the polypeptide capable of binding clusterin is conjugated to a magnetic bead and the step (c) comprises:
(i) contacting the mixture with a magnetic field, and
(ii) recovering a second solution comprising the protein comprising the Fc domain and/or the Fab domain, the second solution being substantially free of the polypeptide capable of binding clusterin and/or the magnetic bead.

4. The method of claim 2, wherein the polypeptide capable of binding clusterin is conjugated to a tag and the and the step (c) comprises:
(i) contacting the solution with a tag-binding agent, optionally wherein the tag-binding agent is immobilized on a solid support, and
(ii) recovering a second solution comprising the protein comprising the Fc domain and/or the Fab domain, the second solution being substantially free of the polypeptide capable of binding clusterin and/or the tag.

5. A method for isolating and/or purifying a protein comprising an Fc domain and/or a Fab domain, the method comprising:
(a) providing a solution comprising the protein comprising the Fc domain and/or the Fab domain, the solution further comprising one or more host cell proteins (HCP),
(b) contacting the solution comprising the protein comprising the Fc domain and/or the Fab domain with a polypeptide immobilized onto the surface of a solid support, wherein the immobilized polypeptide is capable of binding clusterin, and
(c) removing free clusterin, clusterin bound to the polypeptide, and any additional HCPs contemporaneously bound to clusterin and/or the polypeptide from the solution, thereby isolating and/or purifying the protein comprising the Fc domain and/or the Fab domain.

6. The method of claim 5, wherein the step (c) removes at least one HCP, optionally wherein the at least one HCP comprises clusterin and at least one more HCP.

7. The method of any one of claims 4 to 6, wherein the solid support is a bead or a chromatography resin.

8. The method of claim 7, wherein the bead is an agarose bead.

9. The method of claim 8, wherein the agarose bead is an aldehyde-activated agarose bead.

10. The method of any one of claims 7 to 9, wherein the immobilized polypeptide was coupled to the bead via a free NH₂.

11. The method of claim 7, wherein the chromatography resin comprises crosslinked poly[styrene divinylbenzene].

12. The method of any one of claims 1 to 11, wherein the solution comprising the protein comprising the Fc domain and/or the Fab domain is selected from a culture supernatant, an eluate from a chromatography step, and a cell-free extract.

13. The method of claim 12, wherein the chromatography step is selected from an affinity chromatography, an ion exchange chromatography, a hydrophobic interaction chromatography, mixed mode chromatography, a reverse phase chromatography and a size exclusion chromatography.

14. The method of claim 12 or claim 13, wherein the chromatography step is an affinity chromatography.

15. The method of claim 12 or claim 13, wherein the chromatography step is an ion exchange chromatography.

16. The method of claim 12 or claim 13, wherein the chromatography step is a hydrophobic interaction chromatography.

17. The method of claim 12, wherein the culture supernatant is derived from culture of mammalian cells expressing the protein comprising the Fc domain and/or the Fab domain.

18. The method of claim 17, wherein the culture supernatant is derived from culturing a cell line expressing the protein comprising the Fc domain and/or the Fab domain.

19. The method of claim 18, wherein the cell line is selected from NS0 murine myeloma cells, PER.C6 human cells, Chinese hamster ovary (CHO) cells, baby hamster kidney (BHK21) cells, murine myeloma Sp2/0 cells, human embryonic kidney 293 (HEK293) cells, HT-1080 cells, Hela cells, CAP cells, HKB-11 cells, HuH-7 cells, and a derivative thereof.

20. The method of claim 18 or claim 19, wherein the cell line is selected from CHO DUXB11, CHO DG44, CHOK1, ExpiCHO and Expi293.

21. The method of claim 12, wherein the cell-free extract is derived from culture of mammalian cells expressing the protein comprising the Fc domain and/or the Fab domain.

22. The method of claim 21, wherein the cell-free extract is derived from culturing a cell line expressing the protein comprising the Fc domain and/or the Fab domain.

23. The method of claim 22, wherein the cell line is selected from NS0 murine myeloma cells, PER.C6 human cells, Chinese hamster ovary (CHO) cells, baby hamster kidney (BHK21) cells, murine myeloma Sp2/0 cells, human embryonic kidney 293 (HEK293) cells, HT-1080 cells, Hela cells, CAP cells, HKB-11 cells, HuH-7 cells, and a derivative thereof.

24. The method of claim 22 or claim 23, wherein the cell line is selected from CHO DUXB11, CHO DG44, CHOK1, ExpiCHO and Expi293.

25. The method of any one of claims 1 to 24, wherein the protein comprising the Fc domain and/or the Fab domain comprises a mammalian Fc domain.

26. The method of any one of claims 1 to 25, wherein the protein comprising the Fc domain and/or the Fab domain comprises a Fc domain selected from a human Fc domain, a mouse Fc domain, a rat Fc domain, a sheep Fc domain, a goat Fc domain, a donkey Fc domain, a feline Fc domain a hamster Fc domain, a guinea pig Fc domain, a horse Fc domain, a rabbit Fc domain, a pig Fc domain, a dog Fc domain, and a cow Fc domain.

27. The method of claim 25 or claim 26, wherein the protein comprising the Fc domain and/or the Fab domain comprises a human Fc domain, a human Fab domain and/or a humanized Fab domain.

28. The method of any one of claims 25 to 27, wherein the protein comprising the Fc domain and/or the Fab domain comprises a human Fc domain.

29. The method of any one of claims 1 to 28, wherein the protein comprising the Fc domain and/or the Fab domain comprises a Fc domain selected from an IgG Fc domain, an IgA Fc domain an IgM Fc domain, an IgE Fc domain and an IgD Fc domain.

30. The method of claim 29, wherein the IgG Fc domain is selected from an IgG1 Fc domain, an IgG2 Fc domain, an IgG3 Fc domain, and an IgG4 Fc domain.

31. The method of claim 29, wherein the IgA is selected from an IgA1 and an IgA2.

32. The method of claim 1 to 31, wherein the protein comprising the Fc domain and/or the Fab domain is an immunoglobulin.

33. The method of any one of claims 1 to 31, wherein the protein comprising the Fc domain and/or the Fab domain is an antibody, an antibody-like molecule, or a derivative thereof.

34. The method of claim 33, wherein the derivative of the antibody is selected from Fab, Fd, F(ab′)₂, Fab′, and Fv, or a binding fragment thereof.

35. The method of claim 33, wherein the derivative of the antibody-like molecule is selected from a single-domain antibody, a heavy-chain-only antibody (VHH), a single-chain antibody (scFv), scFv, ScFv-Fc, a diabody, a ScFv-CH, a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody or a binding fragment thereof.

36. The method of any one of claims 1 to 31, wherein the protein comprising the Fc domain and/or the Fab domain is a fusion protein.

37. The method of claim 36, wherein the fusion protein is a Fab fusion protein.

38. The method of claim 37, wherein the Fab fusion protein is bispecific or tri-specific.

39. The method of claim 37, wherein the Fab fusion protein is selected from Fab-scFv, Fab-L-scFv, Fab-H-scFv, tribody, Fab-(scFv)2, a TriFab, a Fab-Fab fusion protein, a scFv-Fc-Fab fusion protein, a bispecific T-cell engager (BiTE), a Fab-Fv fusion protein, and a Fab-dsFv fusion protein.

40. The method of claim 36, wherein the fusion protein is an Fc fusion protein.

41. The method of claim 40, wherein the Fc fusion protein comprises a fusion partner selected from single-chain Fv (scFv), single-chain diabody (scDb), Fv, Fab, and F(ab′)2.

42. The method of claim 36, wherein the Fc fusion protein comprises the formula:
(i) X-Fc, wherein X is selected from an antigen, a mammalian intracellular protein, a mammalian membrane protein, a mammalian secreted protein or a fragment thereof;
(ii) Fc-Y, wherein Y is selected from an antigen, a mammalian intracellular protein, a mammalian membrane protein, a mammalian secreted protein or a fragment thereof; or
(iii) X-Fc-Y, wherein X and Y are independently selected from an antigen, a mammalian intracellular protein, a mammalian membrane protein, a mammalian secreted protein or a fragment thereof.

43. The method of claim 42, wherein the X and/or Y is an antigen or a fragment thereof.

44. The method of claim 43, wherein the antigen is derived from a pathogen.

45. The method of claim 44, wherein the pathogen is selected from a virus, a bacterium, a protozoan, a parasite, and a fungus.

46. The method of claim 43, wherein the antigen is a cancer antigen.

47. The method of claim 46, wherein the cancer antigen is a neoantigen.

48. The method of claim 42, wherein the X and/or Y is a mammalian intracellular protein or a fragment thereof.

49. The method of claim 42, wherein the X and/or Y is a mammalian secreted protein or a biologically active fragment thereof.

50. The method of claim 49, wherein the mammalian secreted protein is a coagulation factor, a cytokine or a serum protein.

51. The method of claim 50, wherein the cytokine is selected from IFN-α, IFN-β, IFN-ε, IFN-κ, IFN-w IFN-γ, IL-1α, IL-1β, IL-1, IL-2, IL-3, IL-4, IL-5, IL-6, IL-7, IL-9, IL-10, IL-11, IL-12, IL-13, IL-15, IL-16, IL-17, IL-18, IL-19, IL-20, IL-35, TNF, leukemia inhibitory factor (LIF), oncostatin M (OSM), granulocyte colony-stimulating factor (G-CSF), TNF-α, TNF-β, lymphotoxin (LT)-β, LIGHT, Fas ligand (FasL)/CD178, TGF-β1, TGF-β2, TGF-β3, XCL1, XCL2, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CXCL1, CXCL2, CXCL3, CXCL4, CXCL5, CXCL6, CXCL7, CXCL8, CXCL9, CXCL10, CXCL11, CXCL12, CXCL13, CXCL14, CX3CL1, Epo, Tpo, SCF, and FLT-3L.

52. The method of claim 42, wherein the X and/or Y is a mammalian membrane protein, or a fragment thereof, optionally wherein the mammalian membrane protein is selected from SLAMF4, IL-2 Rα, 4-1BB/TNFRSF9, IL-2R β, ALCAM, B7-1, IL-4 R, B7-H3, BLAME/SLAMFS, CEACAM1, IL-6 R, IL-7 Rα, IL-10Rα, IL-10R β, IL-12Rβ1, IL-12Rβ2, CD2, IL-13Rα1, IL-13, CD3, CD4, ILT2/CDS5j, ILT3/CDS5k, ILT4/CDS5d, ILT5/CDS5a, Integrin α 4/CD49d, CDS, Integrin α E/CD103, CD6, Integrin α M/CD11b, CDS, Integrin α X/CD11c, Integrin β 2/CDIS, KIR/CD15S, CD27/TNFRSF7, KIR2DL1, CD2S, KIR2DL3, CD30/TNFRSFS, KIR2DL4/CD15Sd, CD31/PECAM-1, KIR2DS4, CD40 Ligand/TNFSF5, LAG-3, CD43, LAIR1, CD45, LAIR2, CDS3, Leukotriene B4-R1, CDS4/SLAMF5, NCAM-L1, CD94, NKG2A, CD97, NKG2C, CD229/SLAMF3, NKG2D, CD2F-10/SLAMF9, NT-4, CD69, NTB-A/SLAMF6, Common γ Chain/IL-2 R γ, Osteopontin, CRACC/SLAMF7, PD-1, CRTAM, PSGL-1, CTLA-4, RANK/TNFRSF11A, CX3CR1, CX3CL1, L-Selectin, SIRP β1, SLAM, TCCR/WSX-1, DNAM-1, Thymopoietin, EMMPRIN/CD147, TIM-1, EphB6, TIM-2, Fas/TNFRSF6, TIM-3, Fas Ligand/TNFSF6, TIM-4, Fcγ RIII/CD16, TIM-6, TNFR1/TNFRSF1A, Granulysin, TNF RIII/TNFRSF1B, TRAIL RI/TNFRSFIOA, ICAM-1/CD54, TRAIL R2/TNFRSF10B, ICAM-2/CD102, TRAILR3/TNFRSF10C, IFN-γR1, TRAILR4/TNFRSF10D, IFN-γ R2, TSLP, IL-1 R1 and TSLP R, or a fragment thereof.

53. The method of claim 42, wherein the X and/or Y is independently a mammalian membrane protein, or a fragment thereof.

54. The method of claim 53, wherein the X is a Type I membrane protein, or a fragment thereof.

55. The method of claim 54, wherein the Type I membrane protein is selected from TIM-3, BTLA, PD-1, CTLA-4, LAG-3, CD244, CSF1R, CD160, TIGIT, SIRPα/CD172a, 2B4, VISTA, VSIG8, LAG3, CD200, B7-H3, BTNL3, BTNL8, BTN2A1, BTN3A1, BTN3A2, NKG2A, NKG2B, NKG2C, NKG2D, NKG2E, NKG2F, and NKG2H, and TMIGD2, or a fragment thereof.

56. The method of claim 55, wherein the fragment of the Type I membrane protein is the extracellular domain or the ligand binding portion thereof.

57. The method of claim 53, wherein the Y is a mammalian membrane protein is a Type II membrane protein, or a fragment thereof.

58. The method of claim 57, wherein the Type II membrane protein is selected from OX-40 ligand (OX-40L), LIGHT (CD258), GITR ligand (GITRL), 4-1BB Ligand (4-1BBL), CD70, CD30 ligand (CD30L), CD40 ligand (CD40L), a C-type lectin domain (CLEC) family member, CD137 ligand, RANKL, TRAIL, FasL, TL1A, CD80, CD86, CD58, PD-1, SLAMF6, SIRPα and TGFBR2, or a fragment thereof.

59. The method of claim 58, wherein the fragment of the Type II membrane protein is the extracellular domain or a ligand binding portion thereof.

60. The method of one of claims 42 to 59, wherein the fusion protein is capable of modulating an immune response.

61. The method of any one of claims 42 to 46 or claim 60, wherein the fusion protein is a vaccine.

62. The method of any one of claims 1 to 61, wherein the polypeptide capable of binding clusterin is selected from an antibody, a single-domain antibody, a heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂, or a binding fragment thereof.

63. The method of claim 62, wherein the polypeptide capable of binding clusterin is selected from an antibody, a single-domain antibody, a heavy-chain-only antibody (VHH), a single-chain antibody (scFv), or a binding fragment thereof.

64. The method of claim 62 or claim 63, wherein the polypeptide capable of binding clusterin is a recombinant heavy-chain-only antibody (VHH), or a binding fragment thereof.

65. The method of claim 64, wherein the VHH, or a binding fragment thereof, is a recombinant protein.

66. The method of any one of claims 1 to 65, wherein the method further comprises at least one more purification step.

67. The method of claim 66, wherein the at least one more purification step is liquid chromatography.

68. The method of claim 67, wherein the chromatography is an affinity chromatography.

69. The method of claim 68, wherein the affinity chromatography comprises contacting the solution comprising the protein comprising the Fc domain and/or the Fab domain with a moiety having affinity for the X, the Y, the Fc domain and/or the Fab domain.

70. The method of claim 69, wherein the moiety having affinity for the X and/or the Y is an antibody, or a binding fragment thereof, or an antibody-like molecule or a binding fragment thereof.

71. The method of claim 70, wherein the antibody-like molecule is selected from an antibody, a single-domain antibody, a heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂, or a binding fragment thereof.

72. The method of 69, wherein the moiety having affinity for the Fab domain is an antibody, or a binding fragment thereof, or an antibody-like molecule or a binding fragment thereof.

73. The method of claim 72, wherein the antibody-like molecule is selected from an antibody, a single-domain antibody, a heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂, or a binding fragment thereof.

74. The method of claim 69, wherein the moiety having affinity for the Fc domain is an antibody, or a binding fragment thereof, or an antibody-like molecule or a binding fragment thereof.

75. The method of claim 74, wherein the antibody-like molecule is selected from an antibody, a single-domain antibody, a heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂, or a Fc domain binding fragment thereof.

76. The method of claim 69, wherein the moiety having affinity for the Fc domain is selected from a protein A, a protein G, protein L, protein M, or a derivative thereof.

77. The method of claim 68, wherein the affinity chromatography is selected from FcXL chromatography, protein A chromatography, a protein G chromatography, protein L chromatography, and protein M chromatography.

78. The method of any one of claims 68 to 77, wherein the affinity chromatography comprises:
contacting a solution comprising the protein comprising the Fc domain and/or the Fab domain with the moiety having affinity for the X, the Y, the Fc domain and/or the Fab domain that is conjugated to a solid support, and
thereby directly or indirectly attaching the protein comprising the Fc domain and/or the Fab domain and/or at least one HCP to the solid support.

79. The method of claim 78, wherein the solution comprising the protein comprising the Fc domain and/or the Fab domain is selected from an eluate from a chromatography step, a flow through sample of a chromatography step, a culture supernatant, and a cell-free extract.

80. The method of claim 79, wherein the eluate from the chromatography step is selected from an eluate from an ion exchange chromatography, a hydrophobic interaction chromatography, a reverse phase chromatography and a size exclusion chromatography.

81. The method of claim 79, wherein the flow through sample of the chromatography step is a flow through from a clusterin scavenging chromatography.

82. The method of claim 81, wherein the clusterin is scavenged using a polypeptide immobilized onto the surface of a solid support, wherein the immobilized polypeptide is capable of binding clusterin.

83. The method of claim 82, wherein the polypeptide capable of binding clusterin is selected from an antibody, a single-domain antibody, a heavy-chain-only antibody (VHH), a single-chain antibody (scFv), or a binding fragment thereof.

84. The method of claim 78, wherein the solid support is a bead or a chromatography resin.

85. The method of claim 84, wherein the bead is an agarose bead.

86. The method of claim 85, wherein the agarose bead is an aldehyde-activated agarose bead.

87. The method of any one of claims 84 to 86, wherein the immobilized polypeptide is coupled to the bead via a free NH₂.

88. The method of claim 84, wherein the chromatography resin comprises crosslinked poly[styrene divinylbenzene].

89. The method of any one of claims 69 to 88, wherein the moiety having affinity for the X, the Y, the Fc domain and/or the Fab domain that is conjugated to the solid support is located in a second chromatography column.

90. The method of claim 89, wherein the second chromatography column is washed with a buffer.

91. The method of claim 90, wherein the buffer comprises an ingredient selected from a salt, a detergent, an alcohol, a buffering agent and a combination thereof.

92. The method of claim 91, wherein the buffer has a temperature between 0° C. and 4° C.

93. The method of any one of claims 90 to 92, wherein the wash with the buffer removes the protein comprising the Fc domain and/or the Fab domain and/or the at least one HCP that is indirectly attached to the solid support.

94. The method of any one of claims 68 to 93, further comprising contacting the protein comprising the Fc domain and/or the Fab domain that is directly attached to the solid support with a first elution solution that releases the X, the Y, the Fc domain, and/or the Fab domain from the second chromatography column, thereby forming a first eluate which comprises the protein comprising the Fc domain and/or the Fab domain.

95. The method of any one of claims 68 to 94, wherein the affinity chromatography removes at least one HCP.

96. The method of claim 94, wherein the first eluate comprises fewer contaminants than the solution comprising the protein comprising the Fc domain and/or the Fab domain that was contacted with the moiety having affinity for the X, the Y, the Fc domain and/or the Fab domain that is conjugated to the solid support.

97. The method of claim 96, wherein the contaminants comprise components of a mammalian cell that harbors a nucleic acid that expresses the protein comprising the Fc domain and/or the Fab domain.

98. The method of any one of claims 1 to 97, wherein the method further comprises at least one more purification step.

99. The method of claim 98, wherein the at least one more purification step is liquid chromatography.

100. The method of claim 99, wherein the chromatography is an ion exchange chromatography.

101. The method of claim 100, wherein the chromatography is an anion exchange chromatography or a cation exchange chromatography.

102. The method of claim 99, wherein the ion exchange chromatography comprises:
contacting a solution comprising the protein comprising the Fc domain and/or the Fab domain with an ion exchange matrix that is conjugated to a second solid support,
thereby directly or indirectly attaching the protein comprising the Fc domain and/or the Fab domain and/or at least one HCP to the second solid support.

103. The method of claim 102, wherein the solution comprising the protein comprising the Fc domain and/or the Fab domain is selected from the first eluate, an eluate from a chromatography step, a flow through sample of a chromatography step, a culture supernatant, and a cell-free extract.

104. The method of claim 103, wherein the eluate from the chromatography step is selected from an eluate from a hydrophobic interaction chromatography, a reverse phase chromatography and a size exclusion chromatography.

105. The method of claim 103, wherein the flow through sample of the chromatography step is a flow through from a clusterin scavenging chromatography.

106. The method of claim 105, wherein the clusterin is scavenged using a polypeptide immobilized onto the surface of a solid support, wherein the immobilized polypeptide is capable of binding clusterin.

107. The method of claim 106, wherein the polypeptide capable of binding clusterin is selected from an antibody, a single-domain antibody, a heavy-chain-only antibody (VHH), a single-chain antibody (scFv), or a binding fragment thereof.

108. The method of any one of claims 102 to 107, wherein the ion exchange matrix is selected from a weak carboxymethyl cation-exchanger, a strong sulfopropyl (SP) exchanger, and a weak diethylaminoethyl anion exchanger, and a strong quaternary aminoethyl (QAE) exchanger.

109. The method of any one of claims 102 to 108, wherein the solid support is a bead or a chromatography resin.

110. The method of claim 109, wherein the bead is an agarose bead.

111. The method of claim 110, wherein the agarose bead is an aldehyde-activated agarose bead.

112. The method of claim 111, wherein the immobilized polypeptide is coupled to the bead via a free NH₂.

113. The method of claim 109, wherein the chromatography resin comprises crosslinked poly[styrene divinylbenzene].

114. The method of any one of claims 102 to 113, wherein the ion exchange matrix that is conjugated to the second solid support is located in a third chromatography column.

115. The method of claim 114, wherein the third chromatography column is washed with a buffer.

116. The method of claim 115, wherein the buffer comprises an ingredient selected from a salt, a detergent, an alcohol, a buffering agent and a combination thereof.

117. The method of claim 116, wherein the buffer has a temperature between 0° C. and 4° C.

118. The method of any one of claims 115 to 117, wherein the washing step the third chromatography column removes the protein comprising the Fc domain and/or the Fab domain and/or the at least one HCP that is indirectly attached to the second solid support.

119. The method of anyone of claims 102 to 118, further comprising contacting the protein comprising the Fc domain and/or the Fab domain that is directly attached to the second solid support with a first elution solution that releases the protein comprising the Fc domain and/or the Fab domain from the second chromatography column, thereby forming a second eluate which comprises the protein comprising the Fc domain and/or the Fab domain.

120. The method of any one of claims 102 to 119, wherein the ion exchange chromatography removes at least one HCP.

121. The method of claim 119, wherein the second eluate comprises fewer contaminants than the solution comprising the protein comprising the Fc domain and/or the Fab domain that was contacted with the ion exchange matrix that is conjugated to a second solid support.

122. The method of claim 121, wherein the contaminants comprise components of a mammalian cell that harbors a nucleic acid that expresses the protein comprising the Fc domain and/or the Fab domain.

123. The method of any one of claims 1 to 122, wherein the method further comprises at least one more purification step.

124. The method of claim 123, wherein the at least one more purification step is liquid chromatography.

125. The method of claim 99, wherein the chromatography is a hydrophobic interaction chromatography.

126. The method of claim 125, wherein the hydrophobic interaction chromatography comprises:
contacting a solution comprising the protein comprising the Fc domain and/or the Fab domain with a hydrophobic interaction chromatography (HIC) media that is conjugated to a third solid support,
thereby directly or indirectly attaching the protein comprising the Fc domain and/or the Fab domain and/or at least one HCP to the third solid support.

127. The method of claim 126, wherein the solution comprising the protein comprising the Fc domain and/or the Fab domain is selected from the first eluate, the second eluate, an eluate from a chromatography step, a flow through sample of a chromatography step, a culture supernatant, and a cell-free extract.

128. The method of claim 127, wherein the eluate from the chromatography step is selected from an eluate from a reverse phase chromatography and a size exclusion chromatography.

129. The method of claim 127, wherein the flow through sample of the chromatography step is a flow through from a clusterin scavenging chromatography.

130. The method of claim 129, wherein the clusterin is scavenged using a polypeptide immobilized onto the surface of a solid support, wherein the immobilized polypeptide is capable of binding clusterin.

131. The method of claim 130, wherein the polypeptide capable of binding clusterin is selected from an antibody, a single-domain antibody, a heavy-chain-only antibody (VHH), a single-chain antibody (scFv), or a binding fragment thereof.

132. The method of any one of claims 126 to 131, wherein the HIC media is selected from MACRO-PREP METHYL, MACRO-PREP T-BUTYL (BIO-RAD), CAPTO PHENYL, CAPTO BUTYL (GE Healthcare), TOYOPEARL HIC (Tosoh), FRACTOGEL EMD PHENYL (Merck Millipore) and a membrane adsorber (e.g. SARTOBIND HIC (Sartorius)).

133. The method of any one of claims 126 to 132, wherein the solid support is a bead, a chromatography resin or a membrane.

134. The method of claim 133, wherein the bead is an agarose bead.

135. The method of claim 134, wherein the agarose bead is an aldehyde-activated agarose bead.

136. The method of any one of claims 133 to 135, wherein the immobilized polypeptide is coupled to the bead via a free NH₂.

137. The method of claim 133, wherein the chromatography resin comprises crosslinked poly[styrene divinylbenzene].

138. The method of any one of claims 133 to 137, wherein the HIC media that is conjugated to the third solid support is located in a fourth chromatography column or a membrane.

139. The method of claim 138, wherein the fourth chromatography column or the membrane is washed with a buffer.

140. The method of claim 139, wherein the buffer comprises an ingredient selected from a salt, a detergent, an alcohol, a buffering agent and a combination thereof.

141. The method of claim 140, wherein the buffer has a temperature between 0° C. and 4° C.

142. The method of any one of claims 139 to 141, wherein the washing step the fourth chromatography column removes the protein comprising the Fc domain and/or the Fab domain and/or the at least one HCP that is indirectly attached to the third solid support.

143. The method of any one of claims 133 to 142, further comprising contacting the protein comprising the Fc domain and/or the Fab domain that is directly attached to the third solid support with a first elution solution that releases the protein comprising the Fc domain and/or the Fab domain from the third chromatography column, thereby forming a third eluate which comprises the protein comprising the Fc domain and/or the Fab domain.

144. The method of any one of claims 133 to 143, wherein the hydrophobic interaction chromatography removes at least one HCP.

145. The method of claim 143, wherein the third eluate comprises fewer contaminants than the solution comprising the protein comprising the Fc domain and/or the Fab domain that was contacted with the HIC media that is conjugated to a third solid support.

146. The method of claim 145, wherein the contaminants comprise components of a mammalian cell that harbors a nucleic acid that expresses the protein comprising the Fc domain and/or the Fab domain.

147. A method for isolating and/or purifying a protein comprising an Fc domain and/or a Fab domain, the method comprises
(i) providing a solution comprising the protein comprising the Fc domain and/or the Fab domain, and performing:
(a) a clusterin-scavenging chromatography,
(b) an affinity chromatography,
(c) an ion exchange chromatography, and/or
(d) a hydrophobic interaction chromatography,
and thereby isolating and/or purifying the protein comprising the Fc domain and/or the Fab domain.

148. The method of claim 147, wherein the clusterin-scavenging chromatography comprises:
(a) providing a solution comprising the protein comprising the Fc domain and/or the Fab domain, the solution further comprising one or more host cell proteins (HCP),
(b) contacting the solution comprising the protein comprising the Fc domain and/or the Fab domain with a polypeptide immobilized onto the surface of a solid support, wherein the immobilized polypeptide is capable of binding clusterin, and
(c) recovering the protein that does not bind to the polypeptide immobilized onto the surface of the solid support.

149. The method of claim 147 or claim 148, wherein the affinity chromatography uses a moiety having affinity for the X, the Y, the Fc domain and/or the Fab domain.

150. The method of claim 149, wherein the moiety having affinity for the Fc domain is selected from protein A, a protein G, protein L, and protein M.

151. The method of any one of claims 147 to 150, wherein the ion exchange chromatography is selected from cation exchange chromatography and anion exchange chromatography.

152. A method for isolating and/or purifying a protein comprising an Fc domain and/or a Fab domain, the method comprising:
(i) performing a clusterin-scavenging chromatography comprising:
(a) providing a solution comprising the protein comprising the Fc domain and/or the Fab domain, the solution further comprising one or more host cell proteins (HCP),
(b) contacting the solution comprising the protein comprising the Fc domain and/or the Fab domain with a polypeptide immobilized onto the surface of a solid support, wherein the immobilized polypeptide is capable of binding clusterin, and
(c) recovering the protein that does not bind to the polypeptide immobilized onto the surface of the solid support,
(ii) performing an affinity chromatography selected from FcXL chromatography, protein A chromatography, a protein G chromatography, protein L chromatography, and protein M chromatography,
(iii) performing an ion exchange chromatography selected from cation exchange chromatography and anion exchange chromatography, and/or
(iv) performing a hydrophobic interaction chromatography,
and thereby isolating and/or purifying the protein comprising the Fc domain and/or the Fab domain.

153. The method of any one of claims 147 to 152, wherein the clusterin-scavenging chromatography follows one or more of the affinity chromatography, ion exchange chromatography and hydrophobic interaction chromatography.

154. The method of any one of claims 147 to 152, wherein the clusterin-scavenging chromatography precedes one or more of the affinity chromatography, ion exchange chromatography and hydrophobic interaction chromatography.

155. The method of any one of claims 1 to 66, or 147 to 154, wherein the method removes clusterin and at least one more HCP.

156. The method of claim 155, wherein the at least one more HCP is selected from glyceraldehyde-3-phosphate dehydrogenase (GAPDH), actin (e.g., actin, cytoplasmic 2 isoform X2), and serine protease HTRA1 isoform X2 (HTRA).

157. The method of any one of claims 1 to 155, wherein the method removes at least by 30%, or at least 40%, or at least 40%, or at least 50%, or at least 60%, or at least 70%, or at least 80%, or at least 90% clusterin compared to the solution comprising the protein comprising the Fc domain and/or the Fab domain.

158. The method of any one of claims 155 to 157, wherein the method removes at least by 10%, or at least 20%, or at least 30%, or at least 40%, or at least 50%, or at least 60%, or at least 70% of the at least one more HCP compared to the solution comprising the protein comprising the Fc domain and/or the Fab domain.

159. An isolated and/or purified protein comprising an Fc domain and/or a Fab domain prepared using the method of any one of claims 1 to 158.

160. A composition comprising the isolated and/or purified protein comprising an Fc domain and/or a Fab domain of claim 159.

161. A composition comprising an isolated and/or purified protein comprising an Fc domain and/or a Fab domain prepared using the method of any one of claims 1 to 158.

162. A pharmaceutical composition comprising the isolated and/or purified protein comprising an Fc domain and/or a Fab domain of claim 159, and a pharmaceutically acceptable excipient.

163. A pharmaceutical composition comprising an isolated and/or purified protein comprising an Fc domain and/or a Fab domain prepared using the method of any one of claims 1 to 158, and a pharmaceutically acceptable excipient.

164. A method for treating a cancer or an autoimmune disease in a subject in need thereof, the method comprising administering to the subject a therapeutically-effective amount of the isolated and/or purified protein comprising an Fc domain and/or a Fab domain prepared using the method of any one of claims 1 to 158.

165. A method for treating a cancer or an autoimmune disease in a subject in need thereof, the method comprising administering to the subject a therapeutically-effective amount of the isolated and/or purified protein comprising the Fc domain and/or the Fab domain of claim 159.

166. A method for treating a cancer or an autoimmune disease in a subject in need thereof, the method comprising administering to the subject a therapeutically-effective amount the composition of claim 160 or claim 161.

167. A method for treating a cancer or an autoimmune disease in a subject in need thereof, the method comprising administering to the subject a therapeutically-effective amount of the pharmaceutical composition of claim 162 or claim 163.

168. A method for treating or preventing an infectious disease in a subject in need thereof, the method comprising administering to the subject a therapeutically-effective amount of the isolated and/or purified protein comprising an Fc domain and/or a Fab domain prepared using the method of any one of claims 1 to 158.

169. A method for treating or preventing an infectious disease in a subject in need thereof, the method comprising administering to the subject a therapeutically-effective amount of the isolated and/or purified protein comprising the Fc domain and/or the Fab domain of claim 159.

170. A method for treating or preventing an infectious disease in a subject in need thereof, the method comprising administering to the subject a therapeutically-effective amount the composition of claim 160 or claim 161.

171. A method for treating or preventing an infectious disease in a subject in need thereof, the method comprising administering to the subject a therapeutically-effective amount of the pharmaceutical composition of claim 162 or claim 163.
